(19) United States
(12) Patent Application Publication
(10) Pub. No.: US 2026/0062466 A1
(43) Pub. Date: Mar. 5, 2026

(54) ALPHAVIRUS ANTIGEN BINDING ANTIBODIES AND USES THEREOF

(71) Applicants: WASHINGTON UNIVERSITY, St. Louis, MO (US); EMERGENT TRAVEL HEALTH INC., Redwood City, CA (US)

(72) Inventors: Michael DIAMOND, St. Louis, MO (US); Saravanan RAJU, St. Louis, MO (US); Lucas ADAMS, St. Louis, MO (US); Daved FREMONT, St. Louis, MO (US); Lo VANG, San Diego, CA (US)

(21) Appl. No.: 19/106,870

(22) PCT Filed: Aug. 29, 2023
(86) PCT No.: PCT/US2023/073074

§ 371 Date: Feb. 26, 2025

Related U.S. Application Data
(60) Provisional application No. 63/402,559, filed on Aug. 31, 2022.

(51) Int. Cl. C07K 16/10; A61K 39/00; A61P 31/14

(52) U.S. Cl. CPC C07K 16/116; A61P 31/14; A61K 2039/505; C07K 2317/31; C07K 2317/565; C07K 2317/76; C07K 2317/92

(57) ABSTRACT

The present disclosure provides neutralising human antibodies that binds to an alphavirus. The antibodies are raised against the chikungunya (CHIKV) virus, and show cross-neutralisation and in vivo protection of other alphaviruses.

### FIELD

The present disclosure relates to antigen binding molecules that bind to an alphavirus, compositions comprising the antigen binding molecules, and methods of using the antigen binding molecules or compositions to treat infection caused by an alphavirus.

### BACKGROUND

Alphaviruses are arthropod-transmitted enveloped viruses that can cause arthritis, musculoskeletal disease, or encephalitis. The arthritogenic alphaviruses include Mayaro (MAYV), O'nyong'nyong (ONNV), Ross River (RRV), and chikungunya (CHIKV) viruses, the latter of which has caused several recent epidemics with substantial morbidity in Asia, Africa, Oceania, and the Americas. Although the encephalitic alphaviruses, such as Eastern equine encephalitis (EEEV), Venezuelan equine encephalitis (VEEV), and Western equine encephalitis (WEEV) viruses, cause fewer infections and cases than the arthritogenic alphaviruses, because they spread to the brain, they have greater potential to cause fatal disease. It is a challenge to efficiently treat patients infected by alphaviruses.

### SUMMARY

In some embodiments, the present disclosure provides an antigen binding molecule that binds to an alphavirus. In some embodiments, the antigen binding molecule comprises:
a CDR-H1 comprising SEQ ID NO:2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290 or 298,
a CDR-H2 comprising SEQ ID NO:3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291 or 299, and
a CDR-H3 comprising SEQ ID NO:4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292 or 300; and
a CDR-L1 comprising SEQ ID NO:6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294 or 302,
a CDR-L2 comprising SEQ ID NO:7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295 or 303, and
a CDR-L3 comprising SEQ ID NO: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296 or 304.

In some embodiments, the antigen binding molecule comprises a heavy chain variable region, which comprises an amino acid sequence that is at least 80% identical to a sequence selected from SEQ ID NOs:1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289 and 297; and the antigen binding molecule comprises a light chain variable region, which comprises an amino acid sequence that is at least 80% identical to a sequence selected from SEQ ID NOs:5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293 and 301.

In some embodiments, the heavy chain variable region comprises an amino acid sequence selected from SEQ ID NOs:1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289 and 297; and the light chain variable region comprises an amino acid sequence selected from SEQ ID NOs:5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293 and 301.

In some embodiments, each of CDR-H1, CDR-H2, CDR-H3, CDR-L1, CDR-L2, CDR-L3 is from a same row of Table 1. In some embodiments, each of the CDRs can be a homolog or derivative to a corresponding sequence in Table 1, and the homolog or derivative of a sequence can be a mutation (insertion, deletion, substitute) or modification of the sequence. Table 1 comprises rows 1-38:

TABLE 1
Sets of CDR sequences

| | CDR-H1 | CDR-H2 | CDR-H3 | CDR-L1 | CDR-L2 | CDR-L3 |
|---|---|---|---|---|---|---|
| 1 | SEQ ID NO: 2 | SEQ ID NO: 3 | SEQ ID NO: 4 | SEQ ID NO: 6 | SEQ ID NO: 7 | SEQ ID NO: 8 |
| 2 | SEQ ID NO: 10 | SEQ ID NO: 11 | SEQ ID NO: 12 | SEQ ID NO: 14 | SEQ ID NO: 15 | SEQ ID NO: 16 |
| 3 | SEQ ID NO: 18 | SEQ ID NO: 19 | SEQ ID NO: 20 | SEQ ID NO: 22 | SEQ ID NO: 23 | SEQ ID NO: 24 |
| 4 | SEQ ID NO: 26 | SEQ ID NO: 27 | SEQ ID NO: 28 | SEQ ID NO: 30 | SEQ ID NO: 31 | SEQ ID NO: 32 |
| 5 | SEQ ID NO: 34 | SEQ ID NO: 35 | SEQ ID NO: 36 | SEQ ID NO: 38 | SEQ ID NO: 39 | SEQ ID NO: 40 |
| 6 | SEQ ID NO: 42 | SEQ ID NO: 43 | SEQ ID NO: 44 | SEQ ID NO: 46 | SEQ ID NO: 47 | SEQ ID NO: 48 |
| 7 | SEQ ID NO: 50 | SEQ ID NO: 51 | SEQ ID NO: 52 | SEQ ID NO: 54 | SEQ ID NO: 55 | SEQ ID NO: 56 |
| 8 | SEQ ID NO: 58 | SEQ ID NO: 59 | SEQ ID NO: 60 | SEQ ID NO: 62 | SEQ ID NO: 63 | SEQ ID NO: 64 |
| 9 | SEQ ID NO: 66 | SEQ ID NO: 67 | SEQ ID NO: 68 | SEQ ID NO: 70 | SEQ ID NO: 71 | SEQ ID NO: 72 |
| 10 | SEQ ID NO: 74 | SEQ ID NO: 75 | SEQ ID NO: 76 | SEQ ID NO: 78 | SEQ ID NO: 79 | SEQ ID NO: 80 |
| 11 | SEQ ID NO: 82 | SEQ ID NO: 83 | SEQ ID NO: 84 | SEQ ID NO: 86 | SEQ ID NO: 87 | SEQ ID NO: 88 |
| 12 | SEQ ID NO: 90 | SEQ ID NO: 91 | SEQ ID NO: 92 | SEQ ID NO: 94 | SEQ ID NO: 95 | SEQ ID NO: 96 |
| 13 | SEQ ID NO: 98 | SEQ ID NO: 99 | SEQ ID NO: 100 | SEQ ID NO: 102 | SEQ ID NO: 103 | SEQ ID NO: 104 |
| 14 | SEQ ID NO: 106 | SEQ ID NO: 107 | SEQ ID NO: 108 | SEQ ID NO: 110 | SEQ ID NO: 111 | SEQ ID NO: 112 |
| 15 | SEQ ID NO: 114 | SEQ ID NO: 115 | SEQ ID NO: 116 | SEQ ID NO: 118 | SEQ ID NO: 119 | SEQ ID NO: 120 |
| 16 | SEQ ID NO: 122 | SEQ ID NO: 123 | SEQ ID NO: 124 | SEQ ID NO: 126 | SEQ ID NO: 127 | SEQ ID NO: 128 |
| 17 | SEQ ID NO: 130 | SEQ ID NO: 131 | SEQ ID NO: 132 | SEQ ID NO: 134 | SEQ ID NO: 135 | SEQ ID NO: 136 |
| 18 | SEQ ID NO: 138 | SEQ ID NO: 139 | SEQ ID NO: 140 | SEQ ID NO: 142 | SEQ ID NO: 143 | SEQ ID NO: 144 |
| 19 | SEQ ID NO: 146 | SEQ ID NO: 147 | SEQ ID NO: 148 | SEQ ID NO: 150 | SEQ ID NO: 151 | SEQ ID NO: 152 |
| 20 | SEQ ID NO: 154 | SEQ ID NO: 155 | SEQ ID NO: 156 | SEQ ID NO: 158 | SEQ ID NO: 159 | SEQ ID NO: 160 |
| 21 | SEQ ID NO: 162 | SEQ ID NO: 163 | SEQ ID NO: 164 | SEQ ID NO: 166 | SEQ ID NO: 167 | SEQ ID NO: 168 |
| 22 | SEQ ID NO: 170 | SEQ ID NO: 171 | SEQ ID NO: 172 | SEQ ID NO: 174 | SEQ ID NO: 175 | SEQ ID NO: 176 |
| 23 | SEQ ID NO: 178 | SEQ ID NO: 179 | SEQ ID NO: 180 | SEQ ID NO: 182 | SEQ ID NO: 183 | SEQ ID NO: 184 |
| 24 | SEQ ID NO: 186 | SEQ ID NO: 187 | SEQ ID NO: 188 | SEQ ID NO: 190 | SEQ ID NO: 191 | SEQ ID NO: 192 |
| 25 | SEQ ID NO: 194 | SEQ ID NO: 195 | SEQ ID NO: 196 | SEQ ID NO: 198 | SEQ ID NO: 199 | SEQ ID NO: 200 |
| 26 | SEQ ID NO: 202 | SEQ ID NO: 203 | SEQ ID NO: 204 | SEQ ID NO: 206 | SEQ ID NO: 207 | SEQ ID NO: 208 |
| 27 | SEQ ID NO: 210 | SEQ ID NO: 211 | SEQ ID NO: 212 | SEQ ID NO: 214 | SEQ ID NO: 215 | SEQ ID NO: 216 |
| 28 | SEQ ID NO: 218 | SEQ ID NO: 219 | SEQ ID NO: 220 | SEQ ID NO: 222 | SEQ ID NO: 223 | SEQ ID NO: 224 |
| 29 | SEQ ID NO: 226 | SEQ ID NO: 227 | SEQ ID NO: 228 | SEQ ID NO: 230 | SEQ ID NO: 231 | SEQ ID NO: 232 |
| 30 | SEQ ID NO: 234 | SEQ ID NO: 235 | SEQ ID NO: 236 | SEQ ID NO: 238 | SEQ ID NO: 239 | SEQ ID NO: 240 |
| 31 | SEQ ID NO: 242 | SEQ ID NO: 243 | SEQ ID NO: 244 | SEQ ID NO: 246 | SEQ ID NO: 247 | SEQ ID NO: 248 |
| 32 | SEQ ID NO: 250 | SEQ ID NO: 251 | SEQ ID NO: 252 | SEQ ID NO: 254 | SEQ ID NO: 255 | SEQ ID NO: 256 |
| 33 | SEQ ID NO: 258 | SEQ ID NO: 259 | SEQ ID NO: 260 | SEQ ID NO: 262 | SEQ ID NO: 263 | SEQ ID NO: 264 |
| 34 | SEQ ID NO: 266 | SEQ ID NO: 267 | SEQ ID NO: 268 | SEQ ID NO: 270 | SEQ ID NO: 271 | SEQ ID NO: 272 |
| 35 | SEQ ID NO: 274 | SEQ ID NO: 275 | SEQ ID NO: 276 | SEQ ID NO: 278 | SEQ ID NO: 279 | SEQ ID NO: 280 |
| 36 | SEQ ID NO: 282 | SEQ ID NO: 283 | SEQ ID NO: 284 | SEQ ID NO: 286 | SEQ ID NO: 287 | SEQ ID NO: 288 |
| 37 | SEQ ID NO: 290 | SEQ ID NO: 291 | SEQ ID NO: 292 | SEQ ID NO: 294 | SEQ ID NO: 295 | SEQ ID NO: 296 |
| 38 | SEQ ID NO: 298 | SEQ ID NO: 299 | SEQ ID NO: 300 | SEQ ID NO: 302 | SEQ ID NO: 303 | SEQ ID NO: 304 |

In some embodiments, the heavy chain variable region comprises an amino acid sequence at least 80% identical to SEQ ID NO:1, and the light chain variable region comprises an amino acid sequence at least 80% identical to SEQ ID NO:5;
the heavy chain variable region comprises an amino acid sequence at least 80% identical to SEQ ID NO:9, and the light chain variable region comprises an amino acid sequence at least 80% identical to SEQ ID NO:13;
the heavy chain variable region comprises an amino acid sequence at least 80% identical to SEQ ID NO:17, and the light chain variable region comprises an amino acid sequence at least 80% identical to SEQ ID NO:21;
the heavy chain variable region comprises an amino acid sequence at least 80% identical to SEQ ID NO:25, and the light chain variable region comprises an amino acid sequence at least 80% identical to SEQ ID NO:29;
the heavy chain variable region comprises an amino acid sequence at least 80% identical to SEQ ID NO:33, and the light chain variable region comprises an amino acid sequence at least 80% identical to SEQ ID NO:37;
the heavy chain variable region comprises an amino acid sequence at least 80% identical to SEQ ID NO:41, and the light chain variable region comprises an amino acid sequence at least 80% identical to SEQ ID NO:45;
the heavy chain variable region comprises an amino acid sequence at least 80% identical to SEQ ID NO:49, and the light chain variable region comprises an amino acid sequence at least 80% identical to SEQ ID NO:53;
the heavy chain variable region comprises an amino acid sequence at least 80% identical to SEQ ID NO:57, and the light chain variable region comprises an amino acid sequence at least 80% identical to SEQ ID NO:61;
the heavy chain variable region comprises an amino acid sequence at least 80% identical to SEQ ID NO:65, and the light chain variable region comprises an amino acid sequence at least 80% identical to SEQ ID NO:69;
the heavy chain variable region comprises an amino acid sequence at least 80% identical to SEQ ID NO:73, and the light chain variable region comprises an amino acid sequence at least 80% identical to SEQ ID NO:77;
the heavy chain variable region comprises an amino acid sequence at least 80% identical to SEQ ID NO:81, and the light chain variable region comprises an amino acid sequence at least 80% identical to SEQ ID NO:85;
the heavy chain variable region comprises an amino acid sequence at least 80% identical to SEQ ID NO:89, and the light chain variable region comprises an amino acid sequence at least 80% identical to SEQ ID NO:93;
the heavy chain variable region comprises an amino acid sequence at least 80% identical to SEQ ID NO:97, and the light chain variable region comprises an amino acid sequence at least 80% identical to SEQ ID NO:101;
the heavy chain variable region comprises an amino acid sequence at least 80% identical to SEQ ID NO:105, and the light chain variable region comprises an amino acid sequence at least 80% identical to SEQ ID NO:109;
the heavy chain variable region comprises an amino acid sequence at least 80% identical to SEQ ID NO:113, and the light chain variable region comprises an amino acid sequence at least 80% identical to SEQ ID NO:117;
the heavy chain variable region comprises an amino acid sequence at least 80% identical to SEQ ID NO: 121, and the light chain variable region comprises an amino acid sequence at least 80% identical to SEQ ID NO:125;
the heavy chain variable region comprises an amino acid sequence at least 80% identical to SEQ ID NO:129, and the light chain variable region comprises an amino acid sequence at least 80% identical to SEQ ID NO:133;
the heavy chain variable region comprises an amino acid sequence at least 80% identical to SEQ ID NO: 127, and the light chain variable region comprises an amino acid sequence at least 80% identical to SEQ ID NO:141;
the heavy chain variable region comprises an amino acid sequence at least 80% identical to SEQ ID NO: 145, and the light chain variable region comprises an amino acid sequence at least 80% identical to SEQ ID NO:149;
the heavy chain variable region comprises an amino acid sequence at least 80% identical to SEQ ID NO: 153, and the light chain variable region comprises an amino acid sequence at least 80% identical to SEQ ID NO:157;
the heavy chain variable region comprises an amino acid sequence at least 80% identical to SEQ ID NO: 161, and the light chain variable region comprises an amino acid sequence at least 80% identical to SEQ ID NO:165;
the heavy chain variable region comprises an amino acid sequence at least 80% identical to SEQ ID NO: 169, and the light chain variable region comprises an amino acid sequence at least 80% identical to SEQ ID NO:173;
the heavy chain variable region comprises an amino acid sequence at least 80% identical to SEQ ID NO:177, and the light chain variable region comprises an amino acid sequence at least 80% identical to SEQ ID NO:181;
the heavy chain variable region comprises an amino acid sequence at least 80% identical to SEQ ID NO:185, and the light chain variable region comprises an amino acid sequence at least 80% identical to SEQ ID NO:189;
the heavy chain variable region comprises an amino acid sequence at least 80% identical to SEQ ID NO: 193, and the light chain variable region comprises an amino acid sequence at least 80% identical to SEQ ID NO:197;
the heavy chain variable region comprises an amino acid sequence at least 80% identical to SEQ ID NO:201, and the light chain variable region comprises an amino acid sequence at least 80% identical to SEQ ID NO:205;
the heavy chain variable region comprises an amino acid sequence at least 80% identical to SEQ ID NO:209, and the light chain variable region comprises an amino acid sequence at least 80% identical to SEQ ID NO:213;
the heavy chain variable region comprises an amino acid sequence at least 80% identical to SEQ ID NO:217, and the light chain variable region comprises an amino acid sequence at least 80% identical to SEQ ID NO:221;
the heavy chain variable region comprises an amino acid sequence at least 80% identical to SEQ ID NO:225, and the light chain variable region comprises an amino acid sequence at least 80% identical to SEQ ID NO:229;
the heavy chain variable region comprises an amino acid sequence at least 80% identical to SEQ ID NO:233, and the light chain variable region comprises an amino acid sequence at least 80% identical to SEQ ID NO:237;
the heavy chain variable region comprises an amino acid sequence at least 80% identical to SEQ ID NO:241, and the light chain variable region comprises an amino acid sequence at least 80% identical to SEQ ID NO:245;
the heavy chain variable region comprises an amino acid sequence at least 80% identical to SEQ ID NO:249, and the light chain variable region comprises an amino acid sequence at least 80% identical to SEQ ID NO:253;
the heavy chain variable region comprises an amino acid sequence at least 80% identical to SEQ ID NO:257, and the light chain variable region comprises an amino acid sequence at least 80% identical to SEQ ID NO:261;
the heavy chain variable region comprises an amino acid sequence at least 80% identical to SEQ ID NO:265, and the light chain variable region comprises an amino acid sequence at least 80% identical to SEQ ID NO:269;
the heavy chain variable region comprises an amino acid sequence at least 80% identical to SEQ ID NO:273, and the light chain variable region comprises an amino acid sequence at least 80% identical to SEQ ID NO:277;
the heavy chain variable region comprises an amino acid sequence at least 80% identical to SEQ ID NO:281, and the light chain variable region comprises an amino acid sequence at least 80% identical to SEQ ID NO:285;
the heavy chain variable region comprises an amino acid sequence at least 80% identical to SEQ ID NO:289, and the light chain variable region comprises an amino acid sequence at least 80% identical to SEQ ID NO:293; or
the heavy chain variable region comprises an amino acid sequence at least 80% identical to SEQ ID NO:297, and the light chain variable region comprises an amino acid sequence at least 80% identical to SEQ ID NO:301.

In some embodiments, the antigen binding molecule binds to Chikungunya virus (CHIKV) and at least one additional alphavirus selected from O'nyong'nyong virus (ONNV), Mayaro virus (MAYV), Ross River virus (RRV), Una virus (UNAV), Bebaru virus (BEBV), Getah virus (GETV), Eastern equine encephalitis (EEEV), Venezuelan equine encephalitis (VEEV) and Western equine encephalitis (WEEV).

In some embodiments, the antigen binding molecule binds to CHIKV and ONNV.

In some embodiments, the antigen binding molecule binds to CHIKV and MAYV.

In some embodiments, the antigen binding molecule binds to CHIKV and at least one of UNAV, RRV and BEBV.

In some embodiments, the antigen binding molecule binds to CHIKV, ONNV, MAYV and RRV.

In some embodiments, the antigen binding molecule binds to CHIKV, ONNV, BEBV and EEEV.

In some embodiments, the antigen binding molecule has inhibitory activity of EC₅₀<100 ng/mL measured by focus reduction neutralization tests (FRNTs) against CHIKV-37997 or CHIKV-LR 2006.

In some embodiments, the antigen binding molecule has inhibitory activity of EC₅₀<10 ng/mL measured by FRNTs against CHIKV-37997 or CHIKV-LR 2006.

In some embodiments, the present disclosure provides a pharmaceutical composition comprising the antigen binding molecule described above.

In some embodiments, the present disclosure provides a method of treating a viral infection in a subject in need thereof, comprising administering to the subject a therapeutically effective amount of the antigen binding molecule described above or the pharmaceutical composition described above.

In some embodiments, the viral infection is caused by an alphavirus.

In some embodiments, the alphavirus is Chikungunya virus (CHIKV), O'nyong'nyong virus (ONNV), Mayaro virus (MAYV), Ross River virus (RRV), Una virus (UNAV), Bebaru virus (BEBV), Getah virus (GETV), Eastern equine encephalitis (EEEV), Venezuelan equine encephalitis (VEEV), Western equine encephalitis (WEEV), or a combination thereof.

### BRIEF DESCRIPTION OF THE DRAWINGS

The following drawings form part of the present specification and are included to further demonstrate exemplary embodiments of certain aspects of the present disclosure.

FIG. 1 shows a heatmap of ELISA binding to CHIKV VLP, p62-E1, or E2, and neutralization of CHIKV-LR 2006 by recombinantly expressed mAbs. Intensity of the heatmap corresponds to endpoint titer (N=no detectable binding, 10 μg/mL, 1 μg/mL, and 0.1 μg/mL) for ELISA and binned EC₅₀ values for neutralization. Data are representative of two experiments.

FIGS. 2A-2C show binding by ELISA of selected mAbs against immobilized CHIKV VLP, CHIKV p62-E1, and CHIKV E2, respectively. The results relate to FIG. 1.

FIGS. 3A-3C show characterization of mAbs, and dose-response curves and table of EC₅₀ values of indicated mAbs against CHIKV-37997 and CHIKV-LR 2006 by FRNT. Plots in FIGS. 3A-3C are representative from 4 experiments with EC₅₀ values generated from the mean of all experiments.

FIG. 3D shows a heatmap of relative binding of each mAb (rows) in the presence of reference mouse mAbs or MXRA8-mouse Fc (columns). MAb binding to Expi293 cells expressing CHIKV structural proteins (capsid-E3-E2-6k-E1) was assessed by flow cytometry by measuring the mean fluorescence intensity (MFI). Numbers indicate % MFI in the presence of competitor (mAb or MXRA8-mouse Fc) compared to no competitor.

FIG. 3E shows CHIKV asymmetric unit (PDB: 6NK5) with E1 shown in light gray and capsid shown in purple. E2 is colored by domain with B domain in pale green, A domain in teal, and β-ribbon in royal blue, with the remainder of E2 colored dark gray. The footprints of indicated mAbs are highlighted in red (CHK-265), green (CHK-263), and yellow (CHK-152).

FIG. 3F shows CHIKV asymmetric unit (PDB: 6NK6), colored as in FIG. 3E. Bound MXRA8 is shown in purple.

FIG. 3G shows survival of 4-week-old male C57BL/6J mice treated with 500 μg of anti-interferon alpha and beta receptor subunit 1 (anti-IFNAR1) and indicated anti-CHIKV mAb (100 μg, ˜5 mg/kg) one day prior to inoculation with 10 FFU of CHIKV-LR 2006 via subcutaneous injection. Data are combined from two experiments with n=10 mice per group. Kaplan-Meier survival curve analysis (log-rank test) with Bonferroni correction; **** (p<0.0001).

FIGS. 3H-3J show competition binding analysis by cell-surface expression and immobilization of soluble CHIKV structural proteins.

FIG. 3H and FIG. 3I show flow cytometry gating strategy, where FIG. 3H is for cell-surface competition binding assay with mouse reference mAbs and human mAbs, and FIG. 3I shows representative histogram flow plots of hCHK-265 binding to cells pre-incubated with indicated mouse reference mAb (red) or no antibody control (black).

FIG. 3J shows Bio-Layer Interferometry (BLI) sensograms of indicated mAb captured on anti-human IgG Fc pins dipped into wells containing CHIKV E1 (1 μM). Data are representative of two experiments.

FIG. 3K shows competition ELISA for mAb binding to CHIKV p62-E1. Binding of each biotinylated antibody (rows) in the presence of each blocking antibody (columns) is expressed relative to binding to CHIKV p62-E1 in the absence of a blocking antibody. Data are shown as the mean of two experiments.

FIGS. 4A-4F show exemplary results of epitope mapping of protective mAbs.

FIG. 4A is a table showing percent relative binding of each mAb (columns) against indicated residues mutated to alanine (rows). The value in each cell correspond to % binding compared to wild type (WT) and are the mean of two experiments. Cells colored in blue are residues also identified by neutralization escape.

FIG. 4B shows CHIKV E1-E2 trimer (PDB: 6NK5) surface representation, with E1 shown in light gray, E2 B domain in pale green, E2 A domain in teal, E2 β-ribbon in royal blue, and the remainder of E2 colored dark gray. Alanine scanning hits are colored red. A single E1-E2 heterodimer is outlined in magenta.

FIGS. 4C-4F are ribbon diagrams highlighting alanine scanning hits on E1-E2 heterodimer. Structural proteins are colored as in FIG. 4B. Antibodies are clustered by site, including those binding B domain sites (apex and flank), both B domain and β-ribbon, or A domain. Alanine scanning mutagenesis loss-of-binding residues shared by all mAbs in each cluster are shown in yellow, with others reducing binding of subsets of mAbs shown in red.

FIGS. 4G-4N show protection by mAbs against CHIKV infection in immunodeficient and immunocompetent mice. FIGS. 4G-4I show survival of 4-week-old male C57BL/6J mice treated with 500 μg of anti-IFNAR1 and indicated anti-CHIKV mAb (20 μg, ˜1 mg/kg) one day prior to inoculation with 10 FFU of CHIKV-LR 2006 via a subcutaneous injection. Data are combined from two experiments with n=10 mice per group. Kaplan-Meier survival curve analysis (log-rank test) with Bonferroni correction; **** (p<0.0001).

FIGS. 4J-4N show results from 4-week-old male mice inoculated subcutaneously with 10³ of CHIKV-LR 2006. FIG. 4J showed foot swelling determined by digital caliper measurement at 3 days post infection. FIGS. 4K-4N are virological assessment by qRT-PCR at 3 days post infection in ipsilateral ankle (FIG. 4K) and calf (FIG. 4L), contralateral ankle (FIG. 4M) and calf muscle (FIG. 4N). Data in FIGS. 4J-4N are pooled from three experiments with n=6 to 9 mice per group. * (p<0.05), ** (p<0.01), *** (p<0.001), **** (p<0.0001) by Kruskal-Wallis test and Dunn's post-test correction.

FIGS. 5A-5F show exemplary results of mAbs protection against related arthritogenic alphaviruses.

FIG. 5A is a heatmap displaying normalized MFI of cell-surface binding of mAbs in Vero cells inoculated with the indicated alphaviruses. Normalized MFI was calculated by dividing geometric MFI of each mAb by geometric MFI of hE16 (isotype control). Results are representative of three experiments

FIG. 5B shows flow cytometry plots displaying binding of indicated mAbs (506.A08, 506.C01, and 516.A10) to infected Vero cells. Data is representative of three experiments.

FIG. 5C and FIG. 5D are dose-response curves and mean EC₅₀ values, respectively, of mAbs against indicated arthritogenic alphaviruses by FRNT. Data are representative (FIG. 5C) or the mean values (FIG. 5D) of three experiments.

FIG. 5E and FIG. 5F are viral titers in indicated tissues in 4-week-old C57BL/6 mice administered mAb one day prior to inoculation with 10³ FFU of MAYV (FIG. 5E) or RRV (FIG. 5F). Data are pooled from two experiments with n=7-10 mice per group. * (p<0.05), ** (p<0.01), *** (p<0.001), **** (p<0.0001) by Kruskal-Wallis with Dunn's post-test correction.

FIG. 5G and FIG. 5H show mapping of epitopes by focused arginine scanning and neutralization escape. FIG. 5G is a table showing percent relative binding of each mAb (columns) against indicated residues mutated to arginine or glutamate (rows). Values in each cell correspond to % binding compared to WT and are the mean of two experiments. FIG. 5H is a table of mutated residues identified following neutralization escape against the indicated mAbs.

FIGS. 6A-6I show exemplary results of cryo-EM reconstructions of broadly-neutralizing mAbs and CHK-265 bound to CHIKV VLPs.

FIGS. 6A-6C are whole CHIKV VLP reconstructions with bound Fab fragments colored green (506.A08) (FIG. 6A), yellow (506.C01) (FIG. 6B), or pink (CHK-265) (FIG. 6C), and structural proteins colored radially. Equatorial cross-sections are shown as round insets. Axes of symmetry are designated in FIG. 6A by a pentagon (5-fold; i5), triangles (3-fold; i3), three-pointed stars (quasi-3-fold; q3), and a diamond (2-fold; i2), with axial orientations displayed in the inset

FIGS. 6D-6F show asymmetric unit reconstructions with Fabs colored dark green/lime green (506.A08 heavy/light chain), dark gold/yellow (506.C01 heavy/light chain), or crimson/pink (CHK-265 heavy/light chain). CHIKV E1 is shown in white, and capsid is shown in purple. E2 is colored by domain, with the B domain in light green, A domain in teal, and β-ribbon in royal blue, with the remainder of E2 colored dark gray.

FIG. 6G shows magnified regions from black boxes in FIGS. 6D-6F, colored as in FIGS. 6D-6F, and is a surface representation of E1-E2, with superimposed ribbon diagrams of 506.A08, 506.C01, and CHK-265 Fv fragments.

FIG. 6H is side (left, right) and top (center) views of magnified region from FIGS. 6D-6F, with mAb footprints outlined in green (506.A08), yellow (506.C01), and red (CHK-265). Residue hits from scanning mutagenesis or neutralization escape are labeled, with impacted mAb(s) indicated by colored stars.

FIG. 6I is an alignment of E2 B domains from arthritogenic alphaviruses, including CHIKV-37997, CHIKV-LR 2006, ONNV, GETV, BEBV, RRV, MAYV, and UNAV. Viruses that escape cross-recognition by 506.A08 or 506.C01 are designated by green or yellow diamonds, respectively. Antibody contact residues (determined by Proteins, Interfaces, Structures, and Assemblies (PISA) solvent exclusion analysis) are delineated below the alignment, with scanning mutagenesis or neutralization escape residues designated by stars. Within the alignment, residues implicated in neutralization escape and correlating with loss of cross-recognition are colored magenta.

FIGS. 6J-6N show cross-neutralization of alphaviruses by mAbs. FIGS. 6J-6L are dose-response curves of indicated mAbs, against ONNV by FRNT. FIG. 6M shows dose-response curves of indicated mAbs against SINV-EEEV by FRNT. Here, EEEV-3 is a previously characterized mouse mAb (Kim A S, et al., Protective antibodies against Eastern equine encephalitis virus bind to epitopes in domains A and B of the E2 glycoprotein, Nat Microbiol. 4(1):187-197 (2019)). FIG. 6N shows foot swelling at 3 days post infection following MAYV infection in 4-week-old C57BL/6J male mice treated as prophylaxis with 200 μg of indicated mAbs. The results relate to FIG. 5E. Data are pooled from two experiments with n=7 to 10 mice per group.

FIG. 7 shows cryo-EM data processing pipeline. Flow chart depicting data processing steps for icosahedral and asymmetric unit reconstructions of CHIKV VLP bound to 506.A08, 506.C01, and CHK-265 Fab, respectively.

FIGS. 8A-8I show validation of cryo-EM reconstructions, where FIGS. 8A-8C show local resolution maps of icosahedral reconstructions and gold-standard Fourier shell correlation (GSFSC) curves for the icosahedral reconstructions, FIGS. 8D-8F show local resolution maps of asymmetric unit reconstructions and GSFSC curves for the asymmetric unit reconstructions, and FIGS. 8G-8I show example density and model fits at Fab/E2 interfaces.

FIGS. 9A-9D show structural features of broadly-neutralizing mAbs and CHK-265.

FIGS. 9A-9C are refined models of CHIKV asymmetric unit (upper panels) bound by 506.A08 (FIG. 9A), 506.C01 (FIG. 9B), or CHK-265 (FIG. 9C), with magnified region (lower panels) highlighting the interaction of complementarity-determining regions (CDRs) with E2 B domain. Fv fragments are colored dark green/lime green (506.A08 heavy/light chain), dark gold/yellow (506.C01 heavy/light chain), or crimson/pink (CHK-265 heavy/light chain). CHIKV E1 is shown in white, and capsid is shown in purple. E2 is colored by domain, with the B domain in light green, A domain in teal, and β-ribbon in royal blue, with the remainder of E2 colored dark gray.

FIG. 9D is a model of CHK-265 Fv and neighboring E2, highlighting no or minimal interaction between the framework region of CHK-265 and A domain. The sharpened electron density map is rendered as a black mesh at the interface.

FIG. 10A shows structural comparison of broadly-neutralizing mAbs 506.A08 and 506.C01. FIG. 10B shows structural comparison of the 506.A08, 506.C01 with previously characterized broadly-neutralizing infection-induced mAbs RRV-12 and CHK-265. CHIKV E2 was shown as a surface rendering with B domain (light green) and a portion of E1 (white) visible. Antibody Fv fragments are displayed in dark green/lime green (506.A08 heavy/light chain), dark gold/yellow (506.C01 heavy/light chain), crimson/pink (CHK-265 heavy/light chain), or dark blue/light blue (RRV-12 heavy/light chain; PDB 6W09).

### DETAILED DESCRIPTION

Unless otherwise defined herein, scientific and technical terms used in the present disclosure shall have the meanings that are commonly understood by one of ordinary skill in the art.

Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

Throughout this application, the term “about” is used to indicate that a value includes the inherent variation of error for the method/device being employed to determine the value, or the variation that exists among the study subjects. “About” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

Ranges provided herein, of any type, include all values within a particular range described and values about an endpoint for a particular range. As used herein, “between” is a range inclusive of the ends of the range. For example, a number between x and y explicitly includes the numbers x and y, and any numbers that fall within x and y.

As used herein, the term “substantially,” or “substantial,” when used in a negative connotation refers to the complete or near complete lack of an action, characteristic, property, state, structure, item, or result. For example, a composition that is “substantially” free of a certain component would not have any amount of that component, or the component would be present in such a low amount in the composition that the effect would be the same as if the component were not present.

As used herein, the term “in embodiments” refers to in some embodiments and does not necessarily apply to all embodiments.

As used herein, the term “antigen binding molecule” refers to a molecule (e.g., a polypeptide chain or an assembly of multiple polypeptide chains) that specifically binds an epitope (antigenic determinant) of an antigen. The antigen binding molecule of the disclosure can be monospecific or multi-specific (e.g., bispecific). The antigen binding sites in monospecific binding molecules all bind to the same epitope whereas multispecific binding molecules have at least two antigen-binding sites that bind to different epitopes, which can be on the same or different target molecules. In certain embodiments, the antigen binding molecule in the present disclosure is a functionally active fragment of an antibody, e.g., an IgG (i.e., molecules that contain an antigen binding domain that specifically binds an antigen, also termed antibody fragments or antigen-binding fragments). In some embodiments, the term antigen binding molecule also includes a mutation, such as insertion, deletion, or substitution of amino acids of a reference molecule.

In some embodiments, the antigen binding molecule is an immunoglobulin selected from IgG, IgM, IgD and IgE. In some embodiments, the antigen binding molecule is an IgG. In some embodiment, the antibody comprises a heavy chain constant region selected from an IgG1, an IgG2, IgG3 or an IgG4 isotype, or a variant thereof.

As used herein, the term “antibody” is used in the broadest sense and encompasses various antibody structures, including but not limited to monoclonal antibodies, polyclonal antibodies, and multi-specific antibodies as long as they exhibit the desired antigen-binding activity. In some embodiments, the term antibody as used herein relates to whole (full-length) antibodies (i.e., comprising the elements of two heavy chains and two light chains). In some embodiments, the antibody is monoclonal.

As used herein, the terms “treatment”, “treating” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. Treatment thus covers any treatment of a disease in a mammal, particularly in a human, and includes: (a) preventing the disease from occurring in a subject, i.e., a human, which may contract the disease, but has not yet been diagnosed as having it; (b) inhibiting the disease, e.g., arresting or slowing its development; and (c) relieving the disease, e.g., causing regression of the disease.

The term “subject” is meant any subject, particularly a mammalian subject, in need of treatment with the antigen binding molecule or the pharmaceutical composition comprising the antigen binding molecule. Mammalian subjects include human or non-human animal. In some embodiments, the term “subject” refers to a human subject. In some embodiments, the term “subject” refers to a female subject. In some embodiments, the term “subject” refers to a male subject. In some embodiments, the human subject is 4 years of age or older, 12 years of age or older, 14 years of age or older, or 18 years of age or older.

As used herein, a “subject in need thereof” refers to the subject for whom it is desirable to treat, e.g., a subject having been exposed to an alphavirus.

A non-human animal includes, but is not limited to, pig, dog, cat, guinea pig, rabbit, rat, mouse, horse, cattle, bear, cow, non-human primate, e.g., ape, monkey, orangutan, and chimpanzees, and so on.

A “therapeutically effective amount” refers to the amount of antigen binding molecule according to the present disclosure, when administered to a mammal or other subject for treating a disease, is sufficient to affect such treatment for the disease.

The present disclosure addresses the need for new treatment of alphavirus infection by providing an antigen binding molecule binding an alphavirus and/or an antigen binding molecule with cross-activity against different alphavirus genotypes.

In some embodiments, the antigen binding molecule comprises a VH and VL selected from the below Table 2, or a homolog thereof. In certain embodiments, the antigen binding molecule comprises 3 heavy chain CDRs and 3 light chain CDRs selected from the below Table 2, or a homolog thereof.

TABLE 2

| SEQ ID NO | Sequences | Description |
|---|---|---|
| 1 | QVQLVESGGGVVQPGRSLRLSCAASGFTFSSYGM | 520.B01 VH (Homsap |
| | HWVRQAPGKGLEWVAVISYDGSNKYYADSVKGRF | IGHV3-30*03 F, or |
| | TISRDNSKNTLYLQMNSLRAEDTAVYYCAKGEAW | Homsap IGHV3-30*18 F |
| | FGELSSFDYWGQGTLVTVSS | or Homsap IGHV3-30- |
| | | 5*01 F) |
| 2 | GFTFSSYG | 520.B01 VH CDR-H1 |
| 3 | ISYDGSNK | 520.B01 VH CDR-H2 |
| 4 | AKGEAWFGELSSEDY | 520.B01 VH CDR-H3 |
| 5 | EIVLTQSPGTLSLSPGERATLSCRASQSVSSSYL | 520.B01 VL (Homsap |
| | AWYQQKPGQAPRLLIYGASSRATGIPDRFSGSGS | IGKV3-20*01 F) |
| | GTDFTLTISRLEPEDFAVYYCQQYGSSSFTFGPG | |
| | TKVDIK | |
| 6 | QSVSSSY | 520.B01 VL CDR-L1 |
| 7 | GAS | 520.B01 VL CDR-L2 |
| 8 | QQYGSSSFT | 520.B01 VL CDR-L3 |
| 9 | QVQLVESGGGVVQPGRSLRLSCAASGFTFSNYGI | 520.H01 VH (Homsap |
| | HWVRQAPGKGLEWVAVISYDGTNKYYADSVKGRF | IGHV3-30*03 F, or |
| | AISRDNSKNTLYLRMNSLRAEDTAVYYCAKVVEW | Homsap IGHV3-30*18 F |
| | YCSGGSCYYSGMDVWGQGTTVTVSS | or Homsap IGHV3-30- |
| | | 5*01 F) |
| 10 | GFTFSNYG | 520.H01 VH CDR-H1 |
| 11 | ISYDGTNK | 520.H01 VH CDR-H2 |
| 12 | AKVVEWYCSGGSCYYSGMDV | 520.H01 VH CDR-H3 |
| 13 | DIVMTQSPLSLPVTPGEPASISCRSSQSLLHSNG | 520.H01 VL (Homsap |
| | YNYLDWYLQKPGQSPQLLIYLGSNRASGVPDRFS | IGKV2-28*01 F, or |
| | GSGSGTDFTLKISRVEAEDVGVYYCMQALQTPYT | Homsap IGKV2D-28*01 |
| | FGQGTKLEIK | F) |
| 14 | QSLLHSNGYNY | 520.H01 VL CDR-L1 |
| 15 | LGS | 520.H01 VL CDR-L2 |
| 16 | MQALQTPYT | 520.H01 VL CDR-L3 |
| 17 | QVQLVQSGSELQKPGASVKVSCKASGYTFTENAI | 520. D02 VH (Homsap |
| | IWVRQAPGQGLEWMGRIIRYPGRPTIAQGFTGRF | IGHV7-4-1*02 F) |
| | GFSLDTSINTAYLEISNLKAEDTAFFYCAREYCD | |
| | SSSCSSFDYWGQGTLVTVSS | |
| 18 | GYTFTENA | 520.D02 VH CDR-H1 |
| 19 | IIRYPGRP | 520.D02 VH CDR-H2 |
| 20 | AREYCDSSSCSSFDY | 520.D02 VH CDR-H3 |
| 21 | QSVLTQPPSASGSPGQSVTISCTGINNDVGIYSY | 520.D02 VL (Homsap |
| | VSWYQQHPGKAPKLMIYEVSKRPSGVPDRFSGSK | IGLV2-8*01 F) |
| | SGNTASLTVSGLQADDEADYYCSSYAGSNSYVFG | |
| | SGTKVTVL | |
| 22 | NNDVGIYSY | 520.D02 VL CDR-L1 |
| 23 | EVS | 520.D02 VL CDR-L2 |
| 24 | SSYAGSNSYV | 520.D02 VL CDR-L3 |
| 25 | EVQLVESGGGVVRPGGSLRLSCAASGFTFDDYGM | 520. F02 VH (Homsap |
| | SWVRQAPGKGLEWVSGLNWNGGSTGYADSVKGRF | IGHV3-20*01 F) |
| | TISRDNAKNSLYLQMNSLRAEDTALYHCARMTVV | |
| | AAGTSWYLDLWGRGTLVTVSS | |
| 26 | GFTFDDYG | 520.F02 VH CDR-H1 |
| 27 | LNWNGGST | 520.F02 VH CDR-H2 |
| 28 | ARMTVVAAGTSWYLDL | 520.F02 VH CDR-H3 |
| 29 | DIVLTQSPGTLSLSPGERATLSCRASQSVSSSYL | 520.F02 VL (Homsap |
| | AWYQQKPGQAPRLLIYDASSRATGIPDRFSGSGS | IGKV3-20*01 F) |
| | GTDFTLTISRLEPEDFAVYYCQQYGSSPLDTFGQ | |
| | GTKLEIK | |
| 30 | QSVSSSY | 520.F02 VL CDR-L1 |
| 31 | DAS | 520.F02 VL CDR-L2 |
| 32 | QQYGSSPLDT | 520.F02 VL CDR-L3 |
| 33 | EVQLLESGGGLVQPGGSLRLSCAASGFTFSSYAM | 520.H02 VH (Homsap |
| | SWVRQAPGKGLEWVSAISGSGGSTYYADSVKGRF | IGHV3-23*01 F, or |
| | TISRDNSKNTLYLQMNSLRAEDTAVYYCAKDLGW | Homsap IGHV3-23D*01 |
| | ELLPGSWFDPWGQGTLVTVSS | F) |
| 34 | GFTFSSYA | 520.H02 VH CDR-H1 |
| 35 | ISGSGGST | 520.H02 VH CDR-H2 |
| 36 | AKDLGWELLPGSWFDP | 520.H02 VH CDR-H3 |
| 37 | DIQMTQSPSSLSASVGDRVTITCRASQSISSYLN | 520.H02 VL (Homsap |
| | WYQQKPGKAPKLLIYAASSLQSGVPSRFSGSRSG | IGKV1-39*01 F, or |
| | TDFTLTISSLQPEDFATYYCQQSYSTPYTFGQGT | Homsap IGKV1D-39*01 |
| | KLEIK | F) |
| 38 | QSISSY | 520.H02 VL CDR-L1 |
| 39 | AAS | 520.H02 VL CDR-L2 |
| 40 | QQSYSTPYT | 520.H02 VL CDR-L3 |
| 41 | QVQLVESGGGVVQPGRSLRLSCAASGFTFSSYGM | 520. F04 VH (Homsap |
| | HWVRQAPGKGLEWVAVISYDGSNDYYADSVKGRF | IGHV3-30*03 F, or |
| | TISRDNSKNTLYLQMNSLRAEDTAVYYCAKCVWF | Homsap IGHV3-30*18 F |
| | GELSPFDYWGQGTLVTVSS | or Homsap IGHV3- |
| | | 30*19 F or Homsap |
| | | IGHV3-30-5*01 F or |
| | | Homsap IGHV3-33*05 |
| | | F) |
| 42 | GFTFSSYG | 520.F04 VH CDR-H1 |
| 43 | ISYDGSND | 520.F04 VH CDR-H2 |
| 44 | AKCVWFGELSPFDY | 520.F04 VH CDR-H3 |
| 45 | DIQMTQSPSSLSASVGDRVTITCRASQGISNYFA | 520. F04 VL (Homsap |
| | WYQQKPGKVPKLLIYAASTLQSGVPSRFSGSGSG | IGHV3-30*03 F, or |
| | TDFTLTISSLQPEDVATYYCQKYNSAPGITFGPG | Homsap IGHV3-30*18 F |
| | TKVDIK | or Homsap IGHV3- |
| | | 30*19 F or Homsap |
| | | IGHV3-30-5*01 F or |
| | | Homsap IGHV3-33*05 |
| | | F) |
| 46 | QGISNY | 520. F04 VL CDR-L1 |
| 47 | AAS | 520.F04 VL CDR-L2 |
| 48 | QKYNSAPGIT | 520.F04 VL CDR-L3 |
| 49 | QVQLQESGPGLVKPSQTLSLTCTVSGGSISSGGY | 520.B05 VH (Homsap |
| | YWSWIRQHPGKGLEWIGYIFYSGSTYYNPSLKSR | IGHV4-31*03 F) |
| | VTISVDTSKNQFSLKLSSVTAADTAVYYCARDRG | |
| | GYCFDYWGQGTLVTVSS | |
| 50 | GGSISSGGYY | 520.B05 VH CDR-H1 |
| 51 | IFYSGST | 520.B05 VH CDR-H2 |
| 52 | ARDRGGYCFDY | 520.B05 VH CDR-H3 |
| 53 | DIQMTQSPSSLSASVGDRVTITCQASQDISNYLN | 520.B05 VL (Homsap |
| | WYQQKPGKAPKLLIYDASNLETGVPSRFSGSGSG | IGKV1-33*01 F, or |
| | TDFTFTISSLQPEDIATYYCQQYDNLPPTFGQGT | Homsap IGKV1D-33*01 |
| | KLEIK | F) |
| 54 | QDISNY | 520.B05 VL CDR-L1 |
| 55 | DAS | 520.B05 VL CDR-L2 |
| 56 | QQYDNLPPT | 520.B05 VL CDR-L3 |
| 57 | QVQLQQWGAGLLKPSETLSLTCAVYGGSESGYIL | 520. D05 VH (Homsap |
| | SWIRQPPGKGLEWIGEINHRGSTSYNPSLKSRVT | IGHV4-34*01 F) |
| | ISLDTSKNHFSLKLSSVTAADTAVYYCAKSPYMD | |
| | VWGKGTTVTVSS | |
| 58 | GGSFSGYI | 520.D05 VH CDR-H1 |
| 59 | INHRGST | 520.D05 VH CDR-H2 |
| 60 | AKSPYMDV | 520.D05 VH CDR-H3 |
| 61 | EIVLTQSPGTLSLSPGERATLSCRASQSVSSSYF | 520. D05 VL (Homsap |
| | AWYQQKPGQAPRLLIYGASSRATGIPDRFSGGGS | IGKV3-20*01 F) |
| | GTDFTLTISRLEPEDFAVYYCQQYVSSPYTFGQG | |
| | TKLEIK | |
| 62 | QSVSSSY | 520.D05 VL CDR-L1 |
| 63 | GAS | 520.D05 VL CDR-L2 |
| 64 | QQYVSSPYT | 520.D05 VL CDR-L3 |
| 65 | QVQLVESGGGVVQPGRSLRLSCAASGFTFSSYAM | 520.A06 VH (Homsap |
| | HWVRQAPGKGLEWVALVSYDGSNKYYADSVKGRF | IGHV3-30-3*01 F) |
| | TISRDNSKNTLYLQMNSLRAEDTAVYYCARAEWE | |
| | LGSFDPWGQGTLVTVSS | |
| 66 | GFTFSSYA | 520.A06 VH CDR-H1 |
| 67 | VSYDGSNK | 520.A06 VH CDR-H2 |
| 68 | ARAEWELGSFDP | 520.A06 VH CDR-H3 |
| 69 | EIVLTQSPGTLSLSPGERATLSCRASQSVSSSYL | 520.A06 VL (Homsap |
| | AWYQQKPGQAPRLLIYGASSRATGIPDRLSGSGS | IGKV3-20*01 F) |
| | GTDFTLTISRLEPEDFAVYYCQQYGSSPLTFGQG | |
| | TKVEIK | |
| 70 | QSVSSSY | 520.A06 VL CDR-L1 |
| 71 | GAS | 520.A06 VL CDR-L2 |
| 72 | QQYGSSPLT | 520.A06 VL CDR-L3 |
| 73 | QVQLQESGPGLVKPSGTLSLTCAVSGGSVSSSSW | 520.B06 VH (Homsap |
| | WTWVRQPPGKGLEWIGEIYHSGSTNYNPSLKSRV | IGHV4-4*02 F) |
| | TISVDKSKNQFSLKLISVTAADTAVYYCARRPSG | |
| | SFHFDYWGQGTLVTVSS | |
| 74 | GGSVSSSSW | 520.B06 VH CDR-H1 |
| 75 | IYHSGST | 520.B06 VH CDR-H2 |
| 76 | ARRPSGSFHFDY | 520.B06 VH CDR-H3 |
| 77 | QSALTQPASVSGSPGQSITISCTGTSSDVGGYNY | 520.B06 VL (Homsap |
| | VSWYQQHPGKAPKLMIYDVSNRPSGVSNRFSGSK | IGLV2-14*03 F) |
| | SGNTASLTISGLQAEDEADYYCSSYTSSSTLLVF | |
| | GGGTKLTVL | |
| 78 | SSDVGGYNY | 520.B06 VL CDR-L1 |
| 79 | DVS | 520.B06 VL CDR-L2 |
| 80 | SSYTSSSTLLV | 520.B06 VL CDR-L3 |
| 81 | EVQLVQSGAEVKKPGESLKISCKGSGYSFTSYWI | 516.E07 VH (Homsap |
| | GWVRQMPGKGLEWMGIIYPGDSDTRYSPSFQGQV | IGHV5-51*03 F) |
| | TISADKSISTAYLQWSSLKASDTATYYCARPEHP | |
| | YQYYYGMDVWGQGTTVTVSS | |
| 82 | GYSFTSYW | 516.E07 VH CDR-H1 |
| 83 | IYPGDSDT | 516.E07 VH CDR-H2 |
| 84 | ARPEHPYQYYYGMDV | 516.E07 VH CDR-H3 |
| 85 | QSALTQPRSVSGSPGQSVTISCTGTSSDVGGYNY | 516.E07 VL (Homsap |
| | VSWYQQHPGKAPKLMIYDVSKRPSGVPDRFSGSK | IGLV2-11*01 F) |
| | SGNTASLTISGLQAEDEADYYCCSYSGSYTLVFG | |
| | GGTKLTVL | |
| 86 | SSDVGGYNY | 516.E07 VL CDR-L1 |
| 87 | DVS | 516.E07 VL CDR-L2 |
| 88 | CSYSGSYTLV | 516.E07 VL CDR-L3 |
| 89 | EVQLVQSGAEVKKPGESLKISCKGSGYRFTSYWI | 516. F07 VH (Homsap |
| | AWVRQMPGKGLECMGIIYPGDSDTRYSPSFQGQV | IGHV5-51*01 F) |
| | TISVDKSISTAYLQWSSLKASDTAMFYCARHNNN | |
| | WSLQGYFDYWGQGTLVTVSS | |
| 90 | GYRFTSYW | 516.F07 VH CDR-H1 |
| 91 | IYPGDSDT | 516.F07 VH CDR-H2 |
| 92 | ARHNNNWSLQGYFDY | 516.F07 VH CDR-H3 |
| 93 | PSALTQPASVSGSPGQSITISCTGTSSDVGGYNY | 516. F07 VL (Homsap |
| | VSWYQQHPGKAPKLMIYDVSNRSSGVSNRFSGSK | IGLV2-14*03 F) |
| | SGNTASLTISGLQAEDEADYYCSSYTGSSTSNYV | |
| | FGTGTKVTVL | |
| 94 | SSDVGGYNY | 516.F07 VL CDR-L1 |
| 95 | DVS | 516.F07 VL CDR-L2 |
| 96 | SSYTGSSTSNYV | 516.F07 VL CDR-L3 |
| 97 | EVQLVQSGAEVKKPGESLKISCKGSGYSFTSYWI | 516. G07 VH (Homsap |
| | GWVRQMPGKGLEWMGIIYPGDSDTIYSPSFQGQV | IGHV5-51*03 F) |
| | TISADKSISTAYLQWSSLKASDTAMYYCARQAYS | |
| | SGWAAPSPYWGQGTLVTVSS | |
| 98 | GYSFTSYW | 516.G07 VH CDR-H1 |
| 99 | IYPGDSDT | 516.G07 VH CDR-H2 |
| 100 | ARQAYSSGWAAPSPY | 516.G07 VH CDR-H3 |
| 101 | EIVLTQSPGTLSLSPGKRATLSCRASQSVSSSYL | 516.G07 VL (Homsap |
| | AWYQQKPGQAPRLLIYGASSRATGIPDRFSGSGS | IGKV3-20*01 F) |
| | GTDFTLTISRLEPEDFAVYYCQQYGSSPITFGQG | |
| | TRLEIK | |
| 102 | QSVSSSY | 516.G07 VL CDR-L1 |
| 103 | GAS | 516.G07 VL CDR-L2 |
| 104 | QQYGSSPIT | 516.G07 VL CDR-L3 |
| 105 | EVQLVESGGGLVQPGGSLRLSCAASGFTFSSYEM | 516.H07 VH (Homsap |
| | NWVRQAPGKGLEWVSYMSYTGSTIYYADSVKGRF | IGHV3-48*03 F) |
| | TISRDNGKNSLYLQMNSLRVEDTAVYYCARGGVV | |
| | VAPTYFDYWGQGTLVTVSS | |
| 106 | GFTFSSYE | 516.H07 VH CDR-H1 |
| 107 | MSYTGSTI | 516.H07 VH CDR-H2 |
| 108 | ARGGVVVAPTYFDY | 516.H07 VH CDR-H3 |
| 109 | QSVLTQPPSVSGAPGQRVTISCTGSSSNIGAGYD | 516.H07 VL (Homsap |
| | VHWYQQLPGTAPKLLIYHNNNRPSGVPDRFSASK | IGLV1-40*01 F) |
| | SGTSASLAITGLQAEDEADYYCQSYDSSLSGWVE | |
| | GGGTKLTVL | |
| 110 | SSNIGAGYD | 516.H07 VL CDR-L1 |
| 111 | HNN | 516.H07 VL CDR-L2 |
| 112 | QSYDSSLSGWV | 516.H07 VL CDR-L3 |
| 113 | QVQLAQSGAEVKKPGASVKVSCKASGYIFTSYGI | 516.B08 VH (Homsap |
| | SWVRQAPGQGLEWMGWISAYNSNTNYAQKLQGRV | IGHV1-18*01 F) |
| | TMTTDTSTSTAYMELRSLRSDDTAVYYCARDGSR | |
| | YPAYFDYWGQGTLVTVSS | |
| 114 | GYIFTSYG | 516.B08 VH CDR-H1 |
| 115 | ISAYNSNT | 516.B08 VH CDR-H2 |
| 116 | ARDGSRYPAYFDY | 516.B08 VH CDR-H3 |
| 117 | EIVLTQSPGTLSLSPGERATLSCRASQSVSSSYL | 516.B08 VL (Homsap |
| | AWYQQKPGQAPRLLIYGASSRATGIPDRFSGSGS | IGKV3-20*01 F) |
| | GTDFTLTISRLEPEDFAVYYCQQYDSSPTFGQGT | |
| | KVEIK | |
| 118 | QSVSSSY | 516.B08 VL CDR-L1 |
| 119 | GAS | 516.B08 VL CDR-L2 |
| 120 | QQYDSSPT | 516.B08 VL CDR-L3 |
| 121 | QITLKESGPTLVKPTQTLTLTCTFSGFSLSTSEV | 516.C08 VH (Homsap |
| | GVGWIRQPPGKALEWLALIYWDDDKRYSPSLKSR | IGHV2-5*02 F) |
| | LTITKDTSKNQVVLTMTNMDPVDTATYYCAHIKQ | |
| | DCSSTSCYPVTEDYWGQGTLVTVSS | |
| 122 | GFSLSTSEVG | 516.C08 VH CDR-H1 |
| 123 | IYWDDDK | 516.C08 VH CDR-H2 |
| 124 | AHIKQDCSSTSCYPVTEDY | 516.C08 VH CDR-H3 |
| 125 | DIVMTQSPDSLAVSLGERATINCKSSQSVLDRSN | 516.C08 VL (Homsap |
| | NKKYLAWYQQKSGQPPKLLIYWASTRESGVPDRF | IGKV4-1*01 F) |
| | SGSGSGTDFTLTISSLQAEDVAVYYCQQYYSTPW | |
| | TFGQGTKVEIK | |
| 126 | QSVLDRSNNKKY | 516.C08 VL CDR-L1 |
| 127 | WAS | 516.C08 VL CDR-L2 |
| 128 | QQYYSTPWT | 516.C08 VL CDR-L3 |
| 129 | EVQLVESGGGLVQPGGSLRLSCAASGFTFSSYEM | 516.D08 VH (Homsap |
| | NWVRQAPGKGLEWVSYISSSGSTIYYADSVKGRF | IGHV3-48*03 F) |
| | TISRDNAKNSLYLQMNSLRAEDTAVYYCARGGQD | |
| | EYTSWGVRVGTRRATDRDYYYGMDVWGQGTTVTV | |
| | SS | |
| 130 | GFTFSSYE | 516.D08 VH CDR-H1 |
| 131 | ISSSGSTI | 516.D08 VH CDR-H2 |
| 132 | ARGGQDEYTSWGVRVGTRRATDRDYYYGMDV | 516.D08 VH CDR-H3 |
| 133 | DIQMTQSPSTLSASVGDRVTITCRASQSISSWLA | 516.D08 VL (Homsap |
| | WYQQKPGKAPKLLIYKASSLESGVPSRFSGSGSG | IGKV1-5*03 F) |
| | TEFTLTISSLQPDDFATYYCQQYNTYPLTFGGGT | |
| | KVEIK | |
| 134 | QSISSW | 516.D08 VL CDR-L1 |
| 135 | KAS | 516.D08 VL CDR-L2 |
| 136 | QQYNTYPLT | 516.D08 VL CDR-L3 |
| 137 | EVQLVESGGGLVQPGGSLRLSCAASGFTFSSYEM | 516.E08 VH (Homsap |
| | NWVRQAPGKGLEWVSYISSSDSTIFYADSVKGRF | IGHV3-48*03 F) |
| | TISRDNAKNSLYLQMNSLRAEDTAVYYCARGDPE | |
| | CSGGSCYLWYYYGMDVWGQGTTVTVSS | |
| 138 | GFTFSSYE | 516.E08 VH CDR-H1 |
| 139 | ISSSDSTI | 516.E08 VH CDR-H2 |
| 140 | ARGDPECSGGSCYLWYYYGMDV | 516.E08 VH CDR-H3 |
| 141 | QSVLTQPPSASGTPGQRVTISCSGGSSNIGSNYV | 516.E08 VL (Homsap |
| | YWYQHLPGTAPKLLIYRNNQRPSGVPDRFSGSKS | IGLV1-47*01 F) |
| | GTSASLAISGLRSEDEADYYCAVWDDSLSGWVFG | |
| | GGTKLTVL | |
| 142 | SSNIGSNY | 516.E08 VL CDR-L1 |
| 143 | RNN | 516.E08 VL CDR-L2 |
| 144 | AVWDDSLSGWV | 516.E08 VL CDR-L3 |
| 145 | QVQLVESGGGVVQPGRSLRLSCAASGFTFSSYAM | 516.H08 VH (Homsap |
| | HWVRQAPGKGLEWVAVISYDGSNKYYADSVKGRF | IGHV3-30*04 F, or |
| | TISRDNSKNTLYLQMNSLRAEDTAVYYCARGGKW | Homsap IGHV3-30-3*03 |
| | ELSDFDYWGQGTLVTVSS | F) |
| 146 | GFTFSSYA | 516.H08 VH CDR-H1 |
| 147 | ISYDGSNK | 516.H08 VH CDR-H2 |
| 148 | ARGGKWELSDFDY | 516.H08 VH CDR-H3 |
| 149 | EIVLTQSPGTLSLSPGERATLSCRASQSVSSSYL | 516.H08 VL (Homsap |
| | AWYQQKPGQAPRLLIYGASSRATGIPDRFSGSGS | IGKV3-20*01 F) |
| | GTDFTLTISRLEPEDFAVYYCQQYGSSLFTFGPG | |
| | TKVDIK | |
| 150 | QSVSSSY | 516.H08 VL CDR-L1 |
| 151 | GAS | 516.H08 VL CDR-L2 |
| 152 | QQYGSSLFT | 516.H08 VL CDR-L3 |
| 153 | QVQLVESGGGVVQPGRSLRLSCAASGFTFSSFAM | 516.A09 VH (Homsap |
| | HWVRQAPGKGLEWVAVISYVGSKKYNADSVKGRF | IGHV3-30*04 F, or |
| | TISRDNSKNTLYLQMNSLRAEDTAVYYCARELED | Homsap IGHV3-30-3*03 |
| | YYDSSGYYYGMDVWGQGTTVTVSS | F) |
| 154 | GFTFSSFA | 516.A09 VH CDR-H1 |
| 155 | ISYVGSKK | 516.A09 VH CDR-H2 |
| 156 | ARELEDYYDSSGYYYGMDV | 516.A09 VH CDR-H3 |
| 157 | DIQLTQSPSFLSASVGDRVTITCRASQGISSYLA | 516.A09 VL (Homsap |
| | WYQQKPGKAPKLLIYAASTLQSGVPSRFSGSGSG | IGKV1-9*01 F) |
| | TEFTLTISSLQPEDFATYYCQQLNSYPRTFGPGT | |
| | KVDIK | |
| 158 | QGISSY | 516.A09 VL CDR-L1 |
| 159 | AAS | 516.A09 VL CDR-L2 |
| 160 | QQLNSYPRT | 516.A09 VL CDR-L3 |
| 161 | QVQLVESGGGVVQPGRSLRLSCAASGFTFSSYSM | 516.B09 VH (Homsap |
| | HWVRQAPGKGLEWVAVISYDGSNKYYEDSVKGRF | IGHV3-30*04 F, or |
| | TISRDNSKNTLYLQMHSLRAEDTAVYYCARDKWF | Homsap IGHV3-30-3*03 |
| | GDLNPFDPWGQGTLVTVSS | F) |
| 162 | GFTFSSYS | 516.B09 VH CDR-H1 |
| 163 | ISYDGSNK | 516.B09 VH CDR-H2 |
| 164 | ARDKWFGDLNPFDP | 516.B09 VH CDR-H3 |
| 165 | EIVLTQSPGTLSLSPGERATLSCRASQSVSSSYL | 516.B09 VL (Homsap |
| | AWYQQKPGQAPRLLIYGASSRATGIPDRFSGSGS | IGKV3-20*01 F) |
| | GTDFTLTISRLEPEDFAVYYCQQYGSSPWTFGLG | |
| | TKVEIK | |
| 166 | QSVSSSY | 516.B09 VL CDR-L1 |
| 167 | GAS | 516.B09 VL CDR-L2 |
| 168 | QQYGSSPWT | 516.B09 VL CDR-L3 |
| 169 | QVQLVQSGAEVKKPGSSVKVSCKAFGGTESSSAI | 516. D09 VH (Homsap |
| | TWVRQAPGQGLEWMGRIIPIFGTANYAQKFQGRV | IGHV1-69*14 F, or |
| | TITADKSTTTAYMELSSLRSEDTAVYYCALKADS | Homsap IGHV1-69*15 |
| | YCSGSSCYPSFDYWGQGTLVTVSS | F) |
| 170 | GGTFSSSA | 516.D09 VH CDR-H1 |
| 171 | IIPIFGTA | 516.D09 VH CDR-H2 |
| 172 | ALKADSYCSGSSCYPSFDY | 516.D09 VH CDR-H3 |
| 173 | SYVLTQPPSVSVAPGKTARITCGGNNIGSKSVHW | 516.D09 VL (Homsap |
| | YQQRPGQAPVLVVYDDSDRPSGIPERFSGSNSGN | IGLV3-21*03 F) |
| | TATLTISRVEAGDEADYYCQVWDSSSDQEVFGGG | |
| | TKLTVL | |
| 174 | NIGSKS | 516.D09 VL CDR-L1 |
| 175 | DDS | 516.D09 VL CDR-L2 |
| 176 | QVWDSSSDQEV | 516.D09 VL CDR-L3 |
| 177 | QLQLQESGPGLVKPSETLSLTCTVSGGSISSSSY | 516.H09 VH (Homsap |
| | YWGWIRQPPGKGLEWIGSIYYSGSTYYNPSLKSR | IGHV4-39*07 F) |
| | VTISVDTSKNQFSLKLSSVTAADTAVYYCARHSY | |
| | CSGGSCYLAPLGDWGQGTLVTVSS | |
| 178 | GGSISSSSYY | 516.H09 VH CDR-H1 |
| 179 | IYYSGST | 516.H09 VH CDR-H2 |
| 180 | ARHSYCSGGSCYLAPLGD | 516.H09 VH CDR-H3 |
| 181 | SYVLTQPPSVSVAPGKTARITCGGNNIGSKSVHW | 516.H09 VL (Homsap |
| | YQQKPGQAPVLVIYYDSDRPSGIPERFSGSNSGN | IGLV3-21*04 F) |
| | TATLTISRVEAGDEADYYCQVWDSSSDFYVEGTG | |
| | TKVTVL | |
| 182 | NIGSKS | 516.H09 VL CDR-L1 |
| 183 | YDS | 516.H09 VL CDR-L2 |
| 184 | QVWDSSSDFYV | 516.H09 VL CDR-L3 |
| 185 | QVQMVESGGGVVQPGRSLRLSCAASGFTFSSYPI | 516.A10 VH (Homsap |
| | HWVRQVPGKGLEWVAVISYDGSNKYYADSVKGRF | IGHV3-30*04 F, or |
| | TISRDNSKNTLYLQMNSLRPEDTAVYFCARGRGD | Homsap IGHV3-30-3*03 |
| | FELNSFDYWGQGTLVTVSS | F) |
| 186 | GFTFSSYP | 516.A10 VH CDR-H1 |
| 187 | ISYDGSNK | 516.A10 VH CDR-H2 |
| 188 | ARGRGDFELNSFDY | 516.A10 VH CDR-H3 |
| 189 | EIVLTQSPGTLSLSPGERATLSCRASQSVSSSYL | 516.A10 VL (Homsap |
| | GWYQQKLGQAPRLLIYGASSRATGIPDRFSGSGS | IGKV3-20*01 F) |
| | GTDFTLTISRLEPEDFAVYYCQQYGSTPFTFGPG | |
| | TKVDIK | |
| 190 | QSVSSSY | 516.A10 VL CDR-L1 |
| 191 | GAS | 516.A10 VL CDR-L2 |
| 192 | QQYGSTPFT | 516.A10 VL CDR-L3 |
| 193 | QVQLVESGGGVVQPGRSLRLSCAASGFTFSSYAM | 516.B10 VH (Homsap |
| | HWVRQAPGKGLEWVAIISYDGSNKYYADSVKGRF | IGHV3-30*04 F, or |
| | TISRDNSKNTLYFRMNSLRAEDTAVYYCARAYTE | Homsap IGHV3-30*14 F |
| | LSSLDYWGQGTLVTVSS | or Homsap IGHV3-30- |
| | | 3*03 F) |
| 194 | GFTFSSYA | 516.B10 VH CDR-H1 |
| 195 | ISYDGSNK | 516.B10 VH CDR-H2 |
| 196 | ARAYTELSSLDY | 516.B10 VH CDR-H3 |
| 197 | EIVLTQSPGTLSLSPGERATLSCRASQSFSSSYL | 516.B10 VL (Homsap |
| | AWYQQKPGQAPRLVIYGASSRATGIPDRFSGSGS | IGKV3-20*01 F) |
| | GTDFTLTISRLEPEDFRVYYCQQFGSTPFTFGPG | |
| | TKVDIK | |
| 198 | QSFSSSY | 516.B10 VL CDR-L1 |
| 199 | GAS | 516.B10 VL CDR-L2 |
| 200 | QQFGSTPFT | 516.B10 VL CDR-L3 |
| 201 | EVQLVQSGAEVKKPGESLNISCKTSGYSFTSYWI | 516.G10 VH (Homsap |
| | GWVRQMPGKGLEWMGIIYPGDSDTRYSPSFQGQV | IGHV5-51*01 F) |
| | TISADKSITTAYLQWSSLKASDTAMYYCARFKGV | |
| | GATTLGWFDPWGQGTLVTVSS | |
| 202 | GYSFTSYW | 516.G10 VH CDR-H1 |
| 203 | IYPGDSDT | 516.G10 VH CDR-H2 |
| 204 | ARFKGVGATTLGWFDP | 516.G10 VH CDR-H3 |
| 205 | QSALTQPRSVSGSPGQSVTISCTGTSSDVGGYNY | 516.G10 VL (Homsap |
| | VSWYQQHPGKAPKLMIYDVSKRPSGVPDRFSGSK | IGLV2-11*01 F) |
| | SGNTASLTISGLQAEDEAEYYCCSYTGSYTLVFG | |
| | GGTKLTVL | |
| 206 | SSDVGGYNY | 516.G10 VL CDR-L1 |
| 207 | DVS | 516.G10 VL CDR-L2 |
| 208 | CSYTGSYTLV | 516.G10 VL CDR-L3 |
| 209 | EVQLVQSGAEVKKPGESLKISCKGSGYSFTSYWI | 516.B11 VH (Homsap |
| | GWVRQMPGKGLEWMGIIYPGDSDTRYSPSFQGQV | IGHV5-51*03 F) |
| | SISADKSISTAYLQWSSLKASDTAMYYCARSIPP | |
| | YYPFYFDYWGQGTLVTVSS | |
| 210 | GYSFTSYW | 516.B11 VH CDR-H1 |
| 211 | IYPGDSDT | 516.B11 VH CDR-H2 |
| 212 | ARSIPPYYPFYFDY | 516.B11 VH CDR-H3 |
| 213 | EIVLTQSPGTLSLSPGERATLSCRASQSVSSSYL | 516.B11 VL (Homsap |
| | AWYQQKPGQAPRLLIYGASSRATGIPDRFSGSGS | IGKV3-20*01 F) |
| | GTDFTLTISRLEPEDFAVYYCQQYTSSPNTFGQG | |
| | TKLEIK | |
| 214 | QSVSSSY | 516.B11 VL CDR-L1 |
| 215 | GAS | 516.B11 VL CDR-L2 |
| 216 | QQYTSSPNT | 516.B11 VL CDR-L3 |
| 217 | EVQLVESGGGLVQPGGSLRLSCAASGFTFSSYEM | 516.C11 VH (Homsap |
| | NWVRQAPGKGLEWVSYISSSGSTIYYAESVKGRF | IGHV3-48*03 F) |
| | TISRDNAKNSLYLQMNSLRAEDTAVYYCARGGQD | |
| | EYSSWGVRVGTRRATARDYYYEMDVWGQGTTVTV | |
| | SS | |
| 218 | GFTFSSYE | 516.C11 VH CDR-H1 |
| 219 | ISSSGSTI | 516.C11 VH CDR-H2 |
| 220 | ARGGQDEYSSWGVRVGTRRATARDYYYEMDV | 516.C11 VH CDR-H3 |
| 221 | DIQMTQSPSSLSASVGDRVTITCRASQSISSYLN | 516.C11 VL (Homsap |
| | WYQQKPGKAPKLLIYAASSLQSGVPSRFSGSGSG | IGKV1-39*01 F, or |
| | TDFTLTISSLQPEDIATYYCQQSYSTPRTFGQGT | Homsap IGKV1D-39*01 |
| | KVEIK | F) |
| 222 | QSISSY | 516.C11 VL CDR-L1 |
| 223 | AAS | 516.C11 VL CDR-L2 |
| 224 | QQSYSTPRT | 516.C11 VL CDR-L3 |
| 225 | EVQLVESGGGLVQPGGSLRLSCAASGFTFSSFEM | 516.D11 VH (Homsap |
| | NWVRQAPGKGLEWVSDISSSGSTIYYADSVKGRF | IGHV3-48*03 F) |
| | SISRDNAKNSLYLQMNSLRAEDTAVYYCARGACG | |
| | SGGSCYQTGDLDYWGQGTLVTVSS | |
| 226 | GFTFSSFE | 516.D11 VH CDR-H1 |
| 227 | ISSSGSTI | 516.D11 VH CDR-H2 |
| 228 | ARGACGSGGSCYQTGDLDY | 516.D11 VH CDR-H3 |
| 229 | QAVVTQEPSLTVSPGGTVTLTCGSSSGAVTSGHY | 516.D11 VL (Homsap |
| | PYWFQQKPGQAPRTLIYHTSNKHSWTPARFSGSL | IGLV7-46*01 F) |
| | LGGKAALTLSGAQPEDEAEYYCLLSYSGAWRVFG | |
| | GGTKLTVL | |
| 230 | SGAVTSGHY | 516.D11 VL CDR-L1 |
| 231 | HTS | 516.D11 VL CDR-L2 |
| 232 | LLSYSGAWRV | 516.D11 VL CDR-L3 |
| 233 | QVQLVESGGGVVQPGRSLRLSCAASGFTFSSYGM | 516. F11 VH (Homsap |
| | HWVRQAPGKGLEWVAVIWYDGSNKYYADSVKGRF | IGHV3-33*08 F) |
| | TISRDNSKNTLYLQMNSLRAEDTAVYYCARGPGW | |
| | GGDLDVWGKGTTVTVSS | |
| 234 | GFTFSSYG | 516.F11 VH CDR-H1 |
| 235 | IWYDGSNK | 516.F11 VH CDR-H2 |
| 236 | ARGGQDEYSSW | 516.F11 VH CDR-H3 |
| 237 | SYELTQPLSVSVALGQTARITCGGNNIGSKNVHW | 516. F11 VL (Homsap |
| | YQQKPGQAPVMVIYRDSNRPSGIPERFSGSNSGN | IGLV3-9*01 F) |
| | TATLTISRAQAGDEADYYCQVWDTSTGVFGGGTK | |
| | LTVL | |
| 238 | NIGSKN | 516.F11 VL CDR-L1 |
| 239 | RDS | 516.F11 VL CDR-L2 |
| 240 | QVWDTSTGV | 516.F11 VL CDR-L3 |
| 241 | QVQLVQSGAEVKKPGASVKVSCKASGYTFTGYYM | 516.G11 VH (Homsap |
| | HWVRQAPGQGLEWMGWINPNSGVTNYAQRFQGRV | IGHV1-2*02 F) |
| | TMTRDTSISTASMELSRLRSDDTAVYYCARDDRR | |
| | IVAGADYYAMDVWGQGTTVTVSS | |
| 242 | GYTFTGYY | 516.G11 VH CDR-H1 |
| 243 | INPNSGVT | 516.G11 VH CDR-H2 |
| 244 | ARDDRRIVAGADYYAMDV | 516.G11 VH CDR-H3 |
| 245 | SYELTQPPSVSVSPGQTASITCSGDKLGDKYACW | 516.G11 VL (Homsap |
| | YQQRPGQSPVLVIYQDDKRPSGIPERFSGSNSGN | IGLV3-1*01 F) |
| | AATLTISGTQAIDEADYYCQAWDSSTVVFGGGTK | |
| | LTVL | |
| 246 | KLGDKY | 516.G11 VL CDR-L1 |
| 247 | QDD | 516.G11 VL CDR-L2 |
| 248 | QAWDSSTVV | 516.G11 VL CDR-L3 |
| 249 | EVQLEESGGGLVQPGGSLRLSCAASGFTFSSYWM | 516.B12 VH (Homsap |
| | HWVRQAPGKGLVWVSRINSDGSSTTYADSVKGRF | IGHV3-74*01 F, or |
| | TISRDNAKSTLYLQMNSLRAEDTAVYYCARGGSY | Homsap IGHV3-74*03 |
| | HYSYYFMDVWGKGTTVTVSS | F) |
| 250 | GFTFSSYW | 516.B12 VH CDR-H1 |
| 251 | INSDGSST | 516.B12 VH CDR-H2 |
| 252 | ARGGSYHYSYYFMDV | 516.B12 VH CDR-H3 |
| 253 | SYELTQPPSVSVSPGQTASITCSGDKLGNKYACW | 516.B12 VL (Homsap |
| | YQQKPGQSPVLVICEDRKRPSGIPERFSGSNSGN | IGLV3-1*01 F) |
| | TATLTISGTQAMDEADYYCQAWDSSTALVEGGGT | |
| | KLTVL | |
| 254 | KLGNKY | 516.B12 VL CDR-L1 |
| 255 | EDR | 516.B12 VL CDR-L2 |
| 256 | QAWDSSTALV | 516.B12 VL CDR-L3 |
| 257 | QVQLVESGGGVVQPGRSLRLSCAASGFTFSSYAV | 516.C12 VH (Homsap |
| | HWVRQAPGKGLEWVAVISYDGSNKYYADSVKGRF | IGHV3-30*04 F, or |
| | TISRDNSKNTLSLQMNSLRAEDTAVYHCARGGAV | Homsap IGHV3-30-3*03 |
| | EVAASDYWGQGTLVTVSS | F) |
| 258 | GFTFSSYA | 516.C12 VH CDR-H1 |
| 259 | ISYDGSNK | 516.C12 VH CDR-H2 |
| 260 | ARGGAVEVAASDY | 516.C12 VH CDR-H3 |
| 261 | EIVLTQSPGTLSLSPGERATLSCRASQSVSSSYL | 516.C12 VL (Homsap |
| | AWYQQKPGQAPRLLIYGASSRATGIPDRFSGSGS | IGKV3-20*01 F) |
| | GTDFTLTISRLEPEDFAVYYCQQYGTTPFTFGPG | |
| | TKVDIK | |
| 262 | QSVSSSY | 516.C12 VL CDR-L1 |
| 263 | GAS | 516.C12 VL CDR-L2 |
| 264 | QQYGTTPFT | 516.C12 VL CDR-L3 |
| 265 | QVQLVESGGGVVQPGRSLRLSCAASGFTFSSYGM | 506.A05 VH (Homsap |
| | HWVRQAPGKGLEWLAVISYDGSNKYYADSVKGRF | IGHV3-30*03 F, or |
| | TISRDNSKTTLFLQMNSLRAEDTAVYYCAKDIQR | Homsap IGHV3-30*18 F |
| | RLIDYWGQGTLVTVSS | or Homsap IGHV3-30- |
| | | 5*01 F) |
| 266 | GFTFSSYG | 506.A05 VH CDR-H1 |
| 267 | ISYDGSNK | 506.A05 VH CDR-H2 |
| 268 | AKDIQRRLIDY | 506.A05 VH CDR-H3 |
| 269 | DIQMTQSPSSLSASVGDRVTITCQASQDISNFLN | 506.A05 VL (Homsap |
| | WYQQKPRKAPKLLIFDASNLETGVPSRFSGSGSG | IGKV1-33*01 F, or |
| | TDFTFTISSLQPEDIATYYCQQYDNLPLTFGGGT | Homsap IGKV1D-33*01 |
| | KVEIK | F) |
| 270 | QDISNF | 516.A05 VL CDR-L1 |
| 271 | DAS | 516.A05 VL CDR-L2 |
| 272 | QQYDNLPLT | 516.A05 VL CDR-L3 |
| 273 | EEQLVESGGGLVQPGGSLRLSCAASGFTFSSYSM | 506.A08 VH (Homsap |
| | NWVRQAPGKGLEWVSYISSSSSTIHYADSVKGRF | IGHV3-48*01 F) |
| | TISRDNAKNSLYLQMNSLRAEDTAVYYCAREGNS | |
| | GYEPLDYWGQGTLVTVSS | |
| 274 | GFTFSSYS | 506.A08 VH CDR-H1 |
| 275 | ISSSSSTI | 506.A08 VH CDR-H2 |
| 276 | AREGNSGYEPLDY | 506.A08 VH CDR-H3 |
| 277 | DIQMTQSPSSLSASVGDRVTITCRASQSISSYLN | 506.A08 VL (Homsap |
| | WYQQKPGKAPKLLIYAASSLQSGVPSRFSGSGSG | IGKV1-39*01 F, or |
| | TDFTLTISSLQPEDFATYYCQQSYSTPWTFGQGT | Homsap IGKV1D-39*01 |
| | KVEIK | F) |
| 278 | QSISSY | 516.A08 VL CDR-L1 |
| 279 | AAS | 516.A08 VL CDR-L2 |
| 280 | QQSYSTPWT | 516.A08 VL CDR-L3 |
| 281 | QVQLVESGGGVVQPGRSLRLSCAASGFTFSSYAM | 506.A09 VH (Homsap |
| | HWVRQAPGKGLEWVAVISYDGSNKYYADSVKGRF | IGHV3-30-3*01 F) |
| | TISRDNSKNTLYLQMNSLRAEDTAVYYCARSPWE | |
| | LDAFDIWGQGTMVTVSS | |
| 282 | GFTFSSYA | 506.A09 VH CDR-H1 |
| 283 | ISYDGSNK | 506.A09 VH CDR-H2 |
| 284 | ARSPWELDAFDI | 506.A09 VH CDR-H3 |
| 285 | EIVLTQSPGTLSLSPGERATLSCRASQSVSSSYL | 506.A09 VL (Homsap |
| | AWYQQKPGQAPRLLIYGASSRATGIPDRFSGSGS | IGKV3-20*01 F) |
| | GTDFTLTISRLEPEDFAVYYCQQYGSSQITFGPG | |
| | TKVDIK | |
| 286 | QSVSSSY | 516.A09 VL CDR-L1 |
| 287 | GAS | 516.A09 VL CDR-L2 |
| 288 | QQYGSSQIT | 516.A09 VL CDR-L3 |
| 289 | QVQLQESGPGLVKPSQTLSLTCTVSGGSISSDDY | 506.C01 VH (Homsap |
| | YWTWIRLPPGKGLEWIGYIFYTGGTYYNPSLKSR | IGHV4-30-4*08 (F)) |
| | VTISLDRSKNQFSLKLSSVTAADTAVYFCARAPE | |
| | TYCSTTNCYKGYFDSWGQGTLVTVSS | |
| 290 | GGSISSDDYY | 506.C01 VH CDR-H1 |
| 291 | IFYTGGT | 506.C01 VH CDR-H2 |
| 292 | ARAPETYCSTTNCYKGYFDS | 506.C01 VH CDR-H3 |
| 293 | DIQMTQSPSSVSASVGDRVTITCRASQGISSWLG | 506.C01 VL (Homsap |
| | WYQQKPGKAPKLLIYAASSLQSGVPSRFSGSGSG | IGKV1-12*01 F, or |
| | TDFTLTISSLQPEDFATYYCQQANSFPRTFGQGT | Homsap IGKV1-12*02 F |
| | KVEIK | or Homsap IGKV1D- |
| | | 12*02 F) |
| 294 | QGISSW | 516.C01 VL CDR-L1 |
| 295 | AAS | 516.C01 VL CDR-L2 |
| 296 | QQANSFPRT | 516.C01 VL CDR-L3 |
| 297 | QLQLQESGPGLVKPSETLSLTCTVSGGSISSSSY | 506.C07 VH (Homsap |
| | YWGWIRQPPGKGLEWIGSIYYSGSTYYNPSLKSR | IGHV4-39*01 F) |
| | VTISVDTSKNQFSLKLSSVTAADTAVYYCARDTP | |
| | YCSSTSCYMGDFDYWGQGTLVTVSS | |
| 298 | GGSISSSSYY | 506.C07 VH CDR-H1 |
| 299 | IYYSGST | 506.C07 VH CDR-H2 |
| 300 | ARDTPYCSSTSCYMGDFDY | 506.C07 VH CDR-H3 |
| 301 | DIQMTQSPSSLSASVGDRVTITCRASQSISSYLN | 506.C07 VL (Homsap |
| | WYQQKLGKAPKLLIYAASSLQSGVPSRFSGSGSG | IGKV1-39*01 F, or |
| | TDFTLTISSLQPEDFATYYCQQSYSTPPLFTFGP | Homsap IGKV1D-39*01 |
| | GTKVDIK | F) |
| 302 | QSISSY | 516.C07 VL CDR-L1 |
| 303 | AAS | 516.C07 VL CDR-L2 |
| 304 | QQSYSTPPLFT | 516.C07 VL CDR-L3 |

In certain embodiments, the antigen binding molecule that binds to an alphavirus provided by the present disclosure is an:
a CDR-H1 comprising SEQ ID NO:2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290, 298, or a homolog of any of the above sequences,
a CDR-H2 comprising SEQ ID NO:3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291, 299, or a homolog of any of the above sequences, and
a CDR-H3 comprising SEQ ID NO:4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292, 300, or a homolog of any of the above sequences; and
a CDR-L1 comprising SEQ ID NO:6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294, 302, or a homolog of any of the above sequences,
a CDR-L2 comprising SEQ ID NO:7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295, 303, or a homolog of any of the above sequences, and
a CDR-L3 comprising SEQ ID NO: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296, 304 or a homolog of any of the above sequences.

In some embodiments, a homolog of an amino acid sequence is a mutation such as an insertion, deletion, or substitution of one or more amino acids to the sequence. In some embodiments, a homolog of an amino acid sequence contains conservative amino acid substitutions that would not be expected to substantially change the functionality of the amino acid sequence. Conservative amino acid substitutions include, for example: A for S, T, C, G or V; C for A; D for E, N or S; E for D, Q, S or K; F for M, I, or L; G for A, S or N; H for Y, N, E, Q, or R; I for V, L, M or F; K for R, Q, E or S; L for I, M, V, and F; M for L, I, V or Q; N for S, D, H, Q, E, T, G, H, R or K; Q for E, K, R, D, S, M, H or N; R for K, Q, E or N; S for T, A, N, G, D, E, Q, or K; T for S, N or V; V for I, L, M, T or A; W for Y and F; and Y for F, H or W.

In some embodiments, the antigen binding molecule is an antibody or an antibody derivative. In some embodiments, the antigen binding molecule is an antibody.

In some embodiments, the antibody derivative is an antibody fragment or a chimeric antibody.

In some embodiments, the antibody fragment is Fab, Fab′, F(ab′)₂, Fv, dsFv, or single chain variable fragment (scFv).

In some embodiments, the antigen binding molecule is a monoclonal antibody.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72), the EBV-hybridoma technique (Cole et al., Monoclonal Antibodies and Cancer Therapy, pp 77-96, Alan R Liss, Inc., 1985), and B cell sorting (Pedrioli A et al., Single B cell technologies for monoclonal antibody discovery, Trends Immunol. 42(12): 1143-1158 (2021)).

Antibodies for use in the present disclosure may also be generated using single lymphocyte antibody methods by cloning and expressing immunoglobulin variable region cDNAs generated from single lymphocytes selected for the production of specific antibodies by for example the methods described by Babcook, J. et al, 1996, Proc. Natl. Acad. Sci. USA 93(15): 7843-78481; WO 92/02551; WO 2004/051268 and WO 2004/106377.

The antibodies of the present disclosure can also be generated using various phage display methods known in the art and include those disclosed by Brinkman et al. (in J. Immunol. Methods, 1995, 182:41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. When the antigen-binding molecules disclosed herein are functionally active fragments or derivatives of a whole immunoglobulin such as single chain antibodies, they may be made such as those described in U.S. Pat. No. 4,946,778. Transgenic mice, or other organisms, including other mammals, may be used to express antibodies, including those within the scope of the disclosure.

The antibody of the present disclosure may be chimeric, human or humanized.

Chimeric antibodies are those antibodies encoded by immunoglobulin genes that have been genetically engineered so that the light and heavy chain genes are composed of immunoglobulin gene segments belonging to different species.

Humanized, antibodies are antibody molecules from non-human species having one or more complementarity determining regions (CDRs) from the non-human species and a framework region from a human immunoglobulin molecule (see, e.g., U.S. Pat. No. 5,585,089; WO 91/09967). Preferably the antibody of the present disclosure is humanized. In one embodiment of the present disclosure, there is provided an antibody, for example, a monoclonal antibody.

In humanized antibodies, the heavy and/or light chain contains one or more CDRs (including, if desired, one or more modified CDRs) from a donor antibody (e.g., a murine monoclonal antibody) grafted into a heavy and/or light chain variable region framework of an acceptor antibody (e.g., a human antibody). For a review, see Vaughan et al, Nature Biotechnology, 16, 535-539, 1998. In one embodiment, rather than the entire CDR being transferred, only one or more of the specificity determining residues from any one of the CDRs described herein above are transferred to the human antibody framework (see, for example, Kashmiri et al., 2005, Methods, 36, 25-34). When the CDRs or specificity determining residues are grafted, any appropriate acceptor variable region framework sequence may be used having regard to the class/type of the donor antibody from which the CDRs are derived, including mouse, primate and human framework regions. Preferably, the humanized antibody according to the disclosure comprises a variable domain comprising human acceptor framework regions as well as one or more of the CDRs or specificity determining residues described above.

Examples of human frameworks which can be used in the disclosure are KOL, NEWM, REI, EU, TUR, TEI, LAY and POM (Kabat et al, supra). For example, KOL and NEWM can be used for the heavy chain, REI can be used for the light chain and EU, LAY and POM can be used for both the heavy chain and the light chain. In a CDR-grafted antibody of the disclosure, the acceptor heavy and light chains do not necessarily need to be derived from the same antibody and may, if desired, comprise composite chains having framework regions derived from different chains.

Fully human antibodies are those antibodies in which the variable regions and the constant regions (where present) of both the heavy and the light chains are all of human origin, or substantially identical to sequences of human origin, not necessarily from the same antibody. Examples of fully human antibodies may include antibodies produced for example by the phage display methods described above and antibodies produced by mice in which the murine immunoglobulin variable and constant region genes have been replaced by their human counterparts, e.g., as described in general terms in EP 0546073, U.S. Pat. Nos. 5,545,806, 5,569,825, 5,625,126, 5,633,425, 5,661,016, 5,770,429, EP 0438474 and EP 0463151.

Furthermore, the antibody of the disclosure may comprise a heavy chain constant region selected from an IgG1, an IgG2, an IgG3 or an IgG4 isotype, or a variant thereof. The constant region domains of the antibody of the disclosure, if present, may be selected having regard to the proposed function of the antibody, and in particular the effector functions which may be required. For example, the human IgG constant region domains of the IgG1 and IgG3 isotypes may be used when the antibody effector functions are required. Alternatively, IgG2 and IgG4 isotypes may be used when the antibody effector functions are not required. For example, IgG4 molecules in which the serine at position 241 has been changed to proline as described in Angal et al., Molecular Immunology, 1993, 30 (1), 105-108, may be used. Particularly preferred is the IgG4 constant domain that comprises this change. In some embodiments, the Fc of the antibody of the disclosure is modified to enhance or reduce effector functions, binding to Fc receptors, circulation half-life, or combinations thereof, as discussed, e.g., in Saunders, Frontiers in Immunology, Conceptual Approaches to Modulating Antibody Effector Functions and Circulation Half-Life 10:1296 doi: 10.3389/fimmu.2019.01296 (2019). In another example, glycosylation of Fc can be modified to enhance or reduce effector function as described in, e.g., Maverakis E et al., Glycans in the immune system and the altered glycan theory of autoimmunity: a critical review, J Autoimmun. 57:1-13 (2015).

It should also be appreciated that antigen-binding portions comprised in the antigen binding molecule of the disclosure, such as the functionally-active fragments or derivatives of whole immunoglobulin fragments described above, may be incorporated into other antibody formats than being the antigen-binding portions of the classic IgG format. Alternative format to the classic IgG may include those known in the art and those described herein, such as DVD-Igs, FabFvs for example as disclosed in WO 2009/040562 and WO 2010/035012, diabodies, triabodies, tetrabodies etc. Other examples include a diabody, triabody, tetrabody, bibodies and tribodies (see for example Holliger and Hudson, 2005, Nature Biotech 23(9): 1126-1136; Schoonjans et al. 2001, Biomolecular Engineering, 17 (6), 193-202), nanobodies, tandem scFv, tandem scFv-Fc, FabFv, Fab′Fv, FabdsFv, Fab-scFv, Fab′-scFv, diFab, diFab′, scdiabody, scdiabody-Fc, ScFv-Fc-scFv, scdiabody-CH3, IgG-scFv, scFv-IgG, V-IgG, IgG-V, DVD-Ig, DuoBody, Fab-Fv-Fv, Fab-Fv-Fc and Fab-dsFv-PEG fragments described in WO 2009040562, WO 2010035012, WO 2011/08609, WO 2011/030107 and WO 2011/061492, respectively.

In some embodiments, the term “derivative” includes a molecule that has a chemical modification other than an insertion, deletion, or substitution of amino acids (or nucleic acids). In some embodiments, derivatives comprise covalent modifications, including, but not limited to, chemical bonding with polymers, lipids, or other organic or inorganic moieties. In some embodiments, a chemically modified antigen binding molecule can have a greater circulating half-life than an antigen binding molecule that is not chemically modified. In some embodiments, a chemically modified antigen binding molecule can have improved targeting capacity for desired cells, tissues, and/or organs. In some embodiments, a derivative antigen binding molecule is covalently modified to include one or more water soluble polymer attachments, including, but not limited to, polyethylene glycol, polyoxyethylene glycol, or polypropylene glycol.

Furthermore, in certain embodiments, the antigen binding molecule of the present disclosure comprises at least an additional antigen-binding portion. Therefore, in some embodiments, there is provided an antigen binding molecule, for example, a monoclonal antibody, comprising a first alphavirus antigen-binding portion and further comprises an additional antigen-binding portion.

In some embodiment, the additional antigen-binding portion is capable of increasing, i.e., extending, the half-life of the antibody. In embodiments, the additional antigen-binding portion binds albumin, for example, human serum albumin.

In some embodiments, the antigen binding molecule comprises a heavy chain variable region, which comprises an amino acid sequence that is at least 60%, 70%, 80%, 90%, or at least 95%, or even at least 96%, 97%, 98% or 99% identical to a sequence selected from the following reference sequences: SEQ ID NOs:1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289 and 297; and the antigen binding molecule comprises a light chain variable region, which comprises an amino acid sequence that is at least 60%, 70%, 80%, 90%, or at least 95%, or even at least 96%, 97%, 98% or 99% identical to a sequence selected from the following reference sequences: SEQ ID NOs:5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293 and 301. In some embodiments, the CDR regions of the antigen binding molecule are the same as in the reference sequence. In some embodiments, the CDR regions of the antigen binding molecule only include conservative substitutions.

In some embodiments, the heavy chain variable region comprises an amino acid sequence selected from SEQ ID NOs:1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289 and 297; and the light chain variable region comprises an amino acid sequence selected from SEQ ID NOs:5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293 and 301.

In some embodiments, each of CDR-H1, CDR-H2, CDR-H3, CDR-L1, CDR-L2, CDR-L3 is from a same row of Table 1. In some embodiments, each of the CDR-H1, CDR-H2, CDR-H3, CDR-L1, CDR-L2, CDR-L3 is from an amino acid sequence that is at least 96%, 97%, 98% or 99% identical to its corresponding sequence in Table 1 shown above in the summary section of the present disclosure.

In some embodiments, each of the heavy chain variable region (VH) and the light chain variable region (VL) is from a same row of Table 3 below. For example, the corresponding VH and VL sequence can be SEQ ID NO:1 and SEQ ID NO:5 in row 1 of Table 3. In another example, the corresponding VH and VL sequence can be, for example SEQ ID NO: 297 and SEQ ID NO:301 in row 38 of Table 3. In some embodiments, each of the VH and VL is from an amino acid sequence that is at least 60%, 70%, 80%, 90%, or at least 95%, or at least 96%, 97%, 98% or 99% identical to its corresponding sequence in Table 3. In embodiments, the VH and/or VL sequences having the aforementioned sequence identity are homologs of the sequences of Table 3. In embodiments, the VH and/or VL sequences having the aforementioned sequence identity comprise the identical CDR sequences of the sequences of Table 3.

TABLE 3
Sets of VH and VL sequences

| | VH | VL |
|---|---|---|
| 1 | SEQ ID NO: 1 | SEQ ID NO: 5 |
| 2 | SEQ ID NO: 9 | SEQ ID NO: 13 |
| 3 | SEQ ID NO: 17 | SEQ ID NO: 21 |
| 4 | SEQ ID NO: 25 | SEQ ID NO: 29 |
| 5 | SEQ ID NO: 33 | SEQ ID NO: 37 |
| 6 | SEQ ID NO: 41 | SEQ ID NO: 45 |
| 7 | SEQ ID NO: 49 | SEQ ID NO: 53 |
| 8 | SEQ ID NO: 57 | SEQ ID NO: 61 |
| 9 | SEQ ID NO: 65 | SEQ ID NO: 69 |
| 10 | SEQ ID NO: 73 | SEQ ID NO: 77 |
| 11 | SEQ ID NO: 81 | SEQ ID NO: 85 |
| 12 | SEQ ID NO: 89 | SEQ ID NO: 93 |
| 13 | SEQ ID NO: 97 | SEQ ID NO: 101 |
| 14 | SEQ ID NO: 105 | SEQ ID NO: 109 |
| 15 | SEQ ID NO: 113 | SEQ ID NO: 117 |
| 16 | SEQ ID NO: 121 | SEQ ID NO: 125 |
| 17 | SEQ ID NO: 129 | SEQ ID NO: 133 |
| 18 | SEQ ID NO: 137 | SEQ ID NO: 141 |
| 19 | SEQ ID NO: 145 | SEQ ID NO: 149 |
| 20 | SEQ ID NO: 153 | SEQ ID NO: 157 |
| 21 | SEQ ID NO: 161 | SEQ ID NO: 165 |
| 22 | SEQ ID NO: 169 | SEQ ID NO: 173 |
| 23 | SEQ ID NO: 177 | SEQ ID NO: 181 |
| 24 | SEQ ID NO: 185 | SEQ ID NO: 189 |
| 25 | SEQ ID NO: 193 | SEQ ID NO: 197 |
| 26 | SEQ ID NO: 201 | SEQ ID NO: 205 |
| 27 | SEQ ID NO: 209 | SEQ ID NO: 213 |
| 28 | SEQ ID NO: 217 | SEQ ID NO: 221 |
| 29 | SEQ ID NO: 225 | SEQ ID NO: 229 |
| 30 | SEQ ID NO: 233 | SEQ ID NO: 237 |
| 31 | SEQ ID NO: 241 | SEQ ID NO: 245 |
| 32 | SEQ ID NO: 249 | SEQ ID NO: 253 |
| 33 | SEQ ID NO: 257 | SEQ ID NO: 261 |
| 34 | SEQ ID NO: 265 | SEQ ID NO: 269 |
| 35 | SEQ ID NO: 273 | SEQ ID NO: 277 |
| 36 | SEQ ID NO: 281 | SEQ ID NO: 285 |
| 37 | SEQ ID NO: 289 | SEQ ID NO: 293 |
| 38 | SEQ ID NO: 297 | SEQ ID NO: 301 |

In some embodiments, the antigen binding molecule binds to Chikungunya virus (CHIKV) and at least one additional alphavirus selected from O'nyong'nyong virus (ONNV), Mayaro virus (MAYV), Ross River virus (RRV), Una virus (UNAV), Bebaru virus (BEBV), Eastern equine encephalitis (EEEV), Venezuelan equine encephalitis (VEEV) and Western equine encephalitis (WEEV). CHIKV infection causes a self-limiting febrile illness associated with joint pain, myalgia, and rash. Some patients experience prolonged arthralgia or arthritis months to years after initial infection. The antigen binding molecule according to the present disclosure can be used to inhibit CHIKV infection, reduce infection of CHIKV virus, and treatment of CHIKV infection. Further, antigen binding molecules according to certain embodiments of the disclosure also show cross-activity against one or more alphaviruses in addition to CHIKV, and may be used for preventing or treating different alphavirus diseases.

In some embodiments, the antigen binding molecule binds to both CHIKV and ONNV. In some embodiments, the antigen binding molecule (i) is an antibody selected from 506.A08, 506.A09, 506.C01, 516.A10, 516.B08, 516.B09, 516.B10, 516.C08, 516.C10, 516.C12, 516.D08, 516.E08, 516.H08, 520.A06, 520.B01, 520.B02, 520.D02, 520.D05, 520.F04, 520.H01, and 520.H04, and optionally 516.D09, 516.H07, 516.H09, 520.B03, with reference to Table 2, above, or (ii) comprises the CDRH1, CDRH2, CDRH3, CDRL1, CDRL2 and CDRL3 from any one of the antibodies listed in (i).

In some embodiments, the antigen binding molecule binds to both CHIKV and MAYV. In some embodiments, the antigen binding molecule is (i) an antibody selected from 506.A08, 506.C01, 516.E08, and 520.B02, and optionally 516.H07 and 520.H01, with reference to Table 2, above, or (ii) comprises the CDRH1, CDRH2, CDRH3, CDRL1, CDRL2 and CDRL3 from any one of the antibodies listed in (i).

In some embodiments, the antigen binding molecule binds to CHIKV and at least one of UNAV, RRV and BEBV. In some embodiments, the antigen binding molecule binds to CHIKV and UNAV, and is (i) an antibody selected from 506.C01 and 516.E08, or (ii) comprises the CDRH1, CDRH2, CDRH3, CDRL1, CDRL2 and CDRL3 from any one of the antibodies listed in (i). In some embodiments, the antigen binding molecule binds to CHIKV and RRV, and (i) is an antibody selected from 506.A08 and 520.H04, and optionally 506.C01, or (ii) comprises the CDRH1, CDRH2, CDRH3, CDRL1, CDRL2 and CDRL3 from any one of the antibodies listed in (i). In some embodiments, the antigen binding molecule binds to CHIKV and BEBV, and (i) is an antibody selected from 506.A08, 506.A09, 516.A10 and 520.A06, or (ii) comprises the CDRH1, CDRH2, CDRH3, CDRL1, CDRL2 and CDRL3 from any one of the antibodies listed in (i). In some embodiments, the antigen binding molecule binds to CHIKV, RRV and BEBV, and (i) is an antibody 506.A08, or (ii) comprises the CDRH1, CDRH2, CDRH3, CDRL1, CDRL2 and CDRL3 from any one of the antibodies listed in (i).

In some embodiments, the antigen binding molecule binds to CHIKV, ONNV, MAYV and RRV. In some embodiments, the antigen binding molecule (i) is an antibody selected from 506.A08 and 506.C01, or (ii) comprises the CDRH1, CDRH2, CDRH3, CDRL1, CDRL2 and CDRL3 from any one of the antibodies listed in (i).

In some embodiments, the antigen binding molecule binds to CHIKV, ONNV, BEBV and EEEV. In some embodiments, the antigen binding molecule (i) is the antibody 516.A10, or (ii) comprises the CDRH1, CDRH2, CDRH3, CDRL1, CDRL2 and CDRL3 from antibody 516.A10.

In some embodiments, the antigen binding molecule has inhibitory activity of EC₅₀<100 ng/mL measured by focus reduction neutralization tests (FRNTs) against CHIKV-37997 and/or CHIKV-LR 2006. In some embodiments, the antigen binding molecule has inhibitory activity of EC₅₀<100 ng/mL against CHIKV-37997, and (i) is an antibody selected from 506.A05, 506.A08, 506.A09, 506.C01, 516.C08, 516.F07, 520.A06, 520.D02, 520.D05 and 520.H01, or (ii) comprises the CDRH1, CDRH2, CDRH3, CDRL1, CDRL2 and CDRL3 from any one of the antibodies listed in (i). In some embodiments, the antigen binding molecule has inhibitory activity of EC₅₀<100 ng/mL against CHIKV-LR 2006, and (i) is an antibody selected from 506.A09, 506.C01, 516.A10, 516.C08, 516.F07, 516.H07, 520.A06, 520.B02, 520.D01, 520.D05, 520.F04 and 520.H01, or (ii) comprises the CDRH1, CDRH2, CDRH3, CDRL1, CDRL2 and CDRL3 from any one of the antibodies listed in (i). In some embodiments, the antigen binding molecule has inhibitory activity of EC₅₀<100 ng/ml against both CHIKV-37997 and CHIKV-LR 2006, and (i) is an antibody selected from 506.A09, 506.C01, 516.C08, 516.F07, 520.A06, 520.D02, 520.D05, and 520.H01, or (ii) comprises the CDRH1, CDRH2, CDRH3, CDRL1, CDRL2 and CDRL3 from any one of the antibodies listed in (i).

In some embodiments, the antigen binding molecule has inhibitory activity of EC₅₀<10 ng/ml measured by FRNTs against CHIKV-37997 and/or CHIKV-LR 2006. In some embodiments, the antigen binding molecule has inhibitory activity of EC₅₀<10 ng/ml against CHIKV-37997, and (i) is an antibody selected from 506.A05, 506.C01, 516.F07, 520.D02, and 520.D05, or (ii) comprises the CDRH1, CDRH2, CDRH3, CDRL1, CDRL2 and CDRL3 from any one of the antibodies listed in (i). In some embodiments, the antigen binding molecule has inhibitory activity of EC₅₀<10 ng/mL against CHIKV-LR 2006, and (i) is an antibody selected from 506.C01, 516.A10, 520.A06, and 520.D02, or (ii) comprises the CDRH1, CDRH2, CDRH3, CDRL1, CDRL2 and CDRL3 from any one of the antibodies listed in (i). In some embodiments, the antigen binding molecule has inhibitory activity of EC₅₀<10 ng/ml against both CHIKV-37997 and CHIKV-LR 2006, and (i) is an antibody selected from 506.C01 and 520.D02, or (ii) comprises the CDRH1, CDRH2, CDRH3, CDRL1, CDRL2 and CDRL3 from any one of the antibodies listed in (i).

In some embodiments, the antigen binding molecule binds to at least one of E1 and E2 glycoprotein of a CHIKV virus.

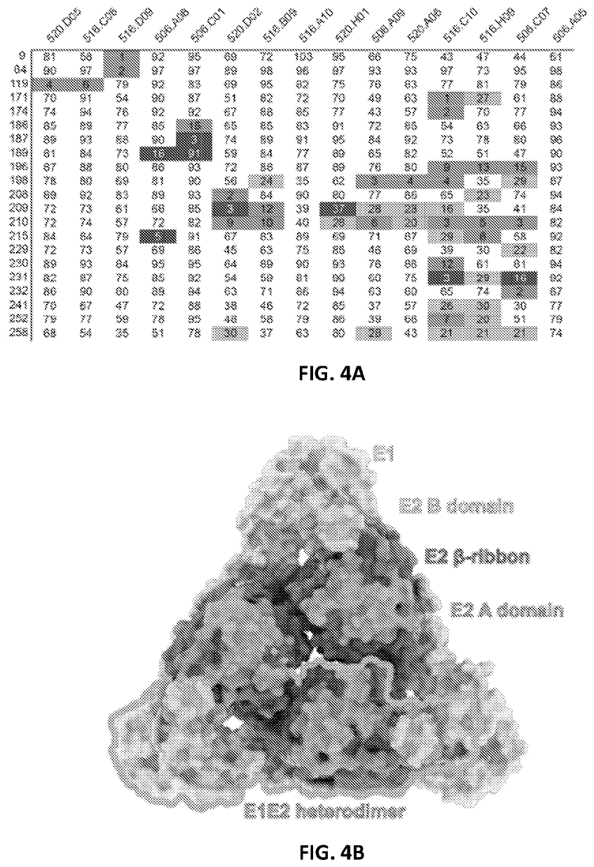

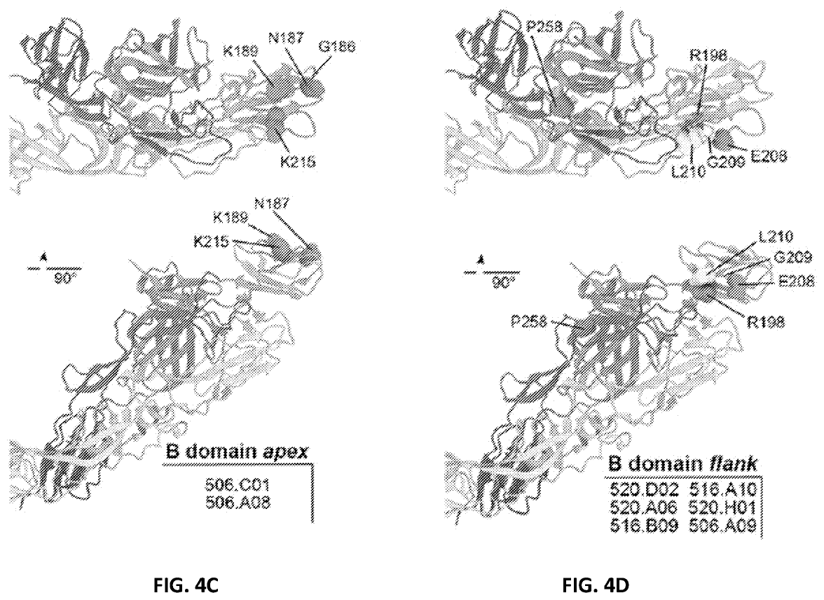

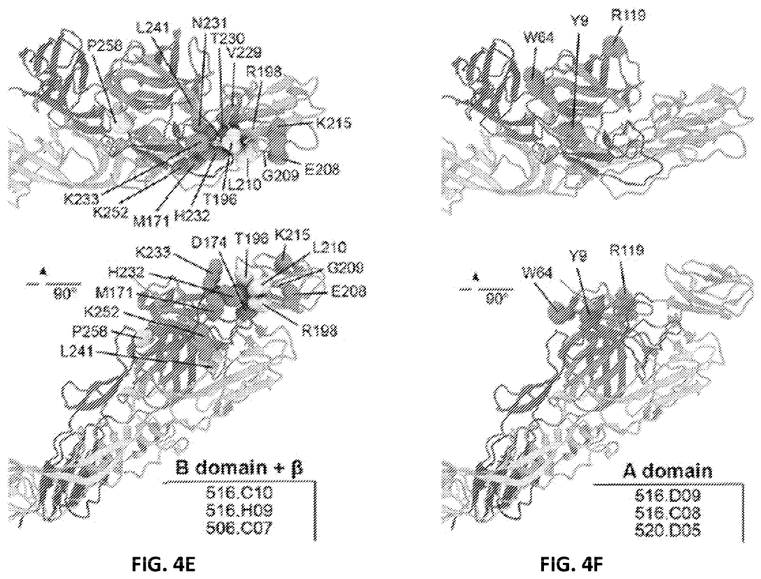

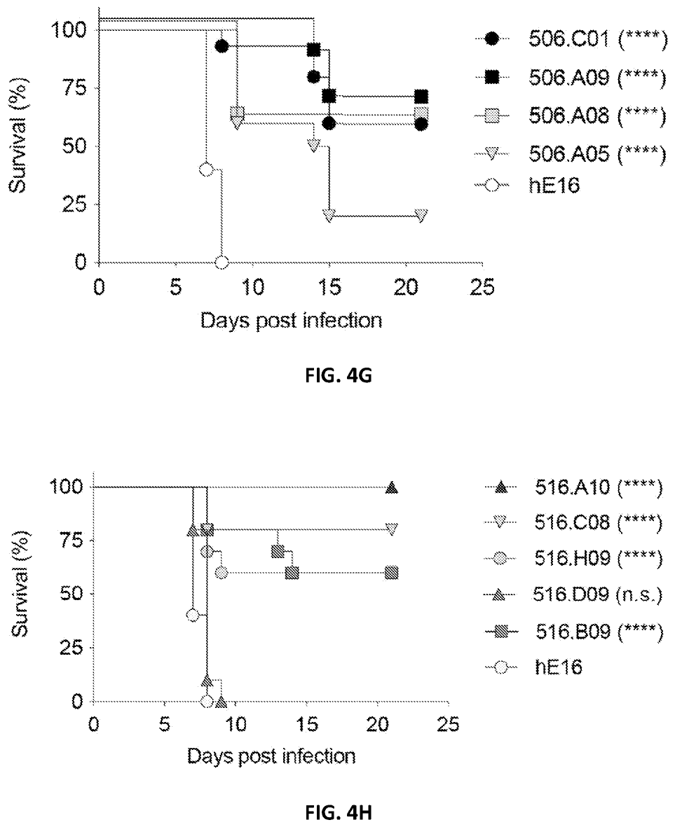

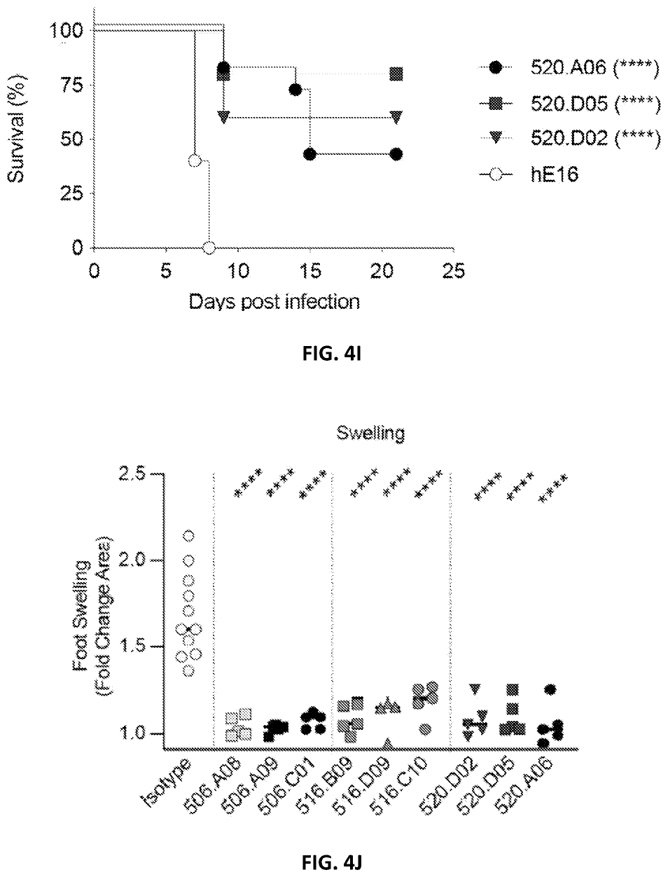

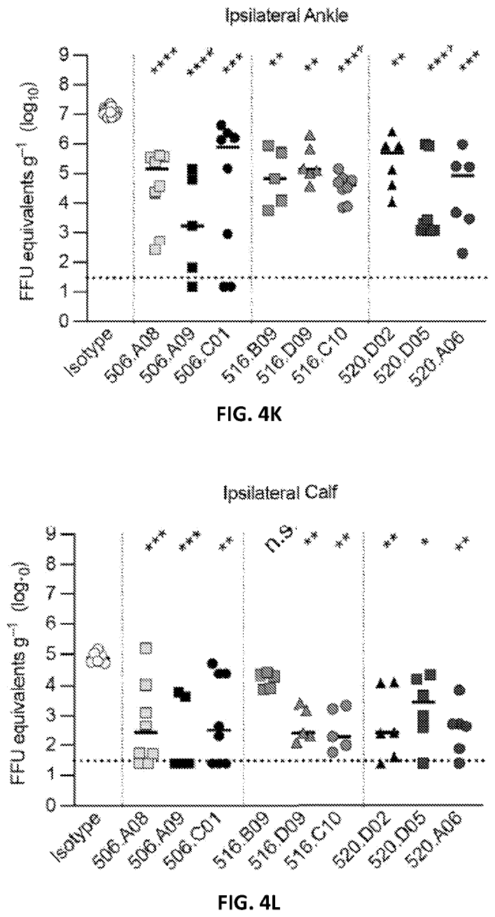

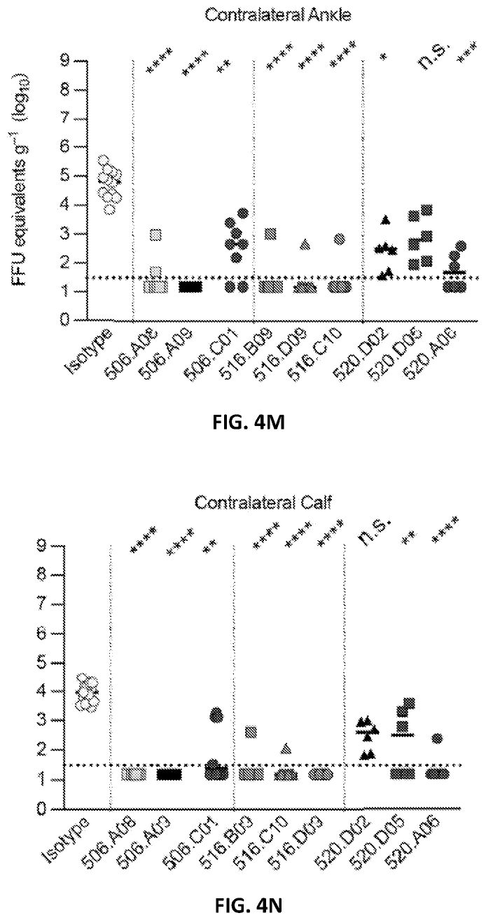

In some embodiments, the antigen binding molecule binds to apex of B domain of the E2 glycoprotein. In some embodiments, the antigen binding molecule binds at least one of G186, N187, K189 and K215 of the E2 glycoprotein. In some embodiments, the antigen binding molecule is an antibody of 506.A08 or 506.C01. As shown in FIG. 4A and FIG. 4C, 506.A08 binds at least to residues K189 and K215 of the E2 glycoprotein, and 506.C01 binds at least to residues G186, N187 and K189 of the E2 glycoprotein.

In some embodiments, the antigen binding molecule binds to a flank of B domain of the E2 glycoprotein. In some embodiments, the antigen binding molecule binds at least one of R198, E208, G209, L210 and P258 of the E2 glycoprotein. In some embodiments, the antigen binding molecule is (i) an antibody of 506.A09, 516.A10, 516.B09, 520.A06, 520.D02, or 520.H01, or (ii) comprises the CDRH1, CDRH2, CDRH3, CDRL1, CDRL2 and CDRL3 from any one of the antibodies listed in (i). As shown in FIG. 4A and FIG. 4D, 506.A09 binds to at least residues R198, G209, L210 and P258 of the E2 glycoprotein; 516.B09 binds to at least residues R198, G209 and L210 of the E2 glycoprotein; 520.A06 binds to at least residues R198, G209 and L210 of the E2 glycoprotein; 520.D02 binds to at least residues E208, G209, L210 and P258 of the E2 glycoprotein; and 520.H01 binds to at least residues G209 and L210 of the E2 glycoprotein.

In some embodiments, the antigen binding molecule binds to B domain and β-ribbon connecting A and the B domains of the E2 glycoprotein. In some embodiments, the antigen binding molecule binds at least one of M171, D174, T196, R198, E208, G209, L210, K215, V229, T230, N231, H232, K233, L241, K252 and P258 of the E2 glycoprotein. In some embodiments, the antigen binding molecule (i) is an antibody of 516.C10, 516. H09 or 506.C07, or (ii) comprises the CDRH1, CDRH2, CDRH3, CDRL1, CDRL2 and CDRL3 from any one of the antibodies listed in (i). As shown in FIG. 4A and FIG. 4E, 506.C07 at least binds to residues T196, R198, L210, V229, N231, H232, and P258; 516.C10 at least binds to residues M171, D174, T196, R198, G209, L210, K215, T230, N231, L241, K252 and P258 of the E2 glycoprotein; and 516. H09 at least binds to residues M171, T196, E208, L210, K215, N231, L241, K252 and P258 of the E2 glycoprotein.

In some embodiments, the antigen binding molecule binds to A domain of the E2 glycoprotein. In some embodiments, the antigen binding molecule binds at least one of Y9, W64 and R119 of the E2 glycoprotein. In some embodiments, the antigen binding molecule (i) is an antibody of 516.C08, 516.D09 and 520.D05, or (ii) comprises the CDRH1, CDRH2, CDRH3, CDRL1, CDRL2 and CDRL3 from any one of the antibodies listed in (i). As shown in FIG. 4A and FIG. 4F, 516.C08 at least binds to residue R119 of the E2 glycoprotein; 516.D09 at least binds to residues Y9 and W64 of the E2 glycoprotein; and 520.D05 at least binds to residue R119 of the E2 glycoprotein.

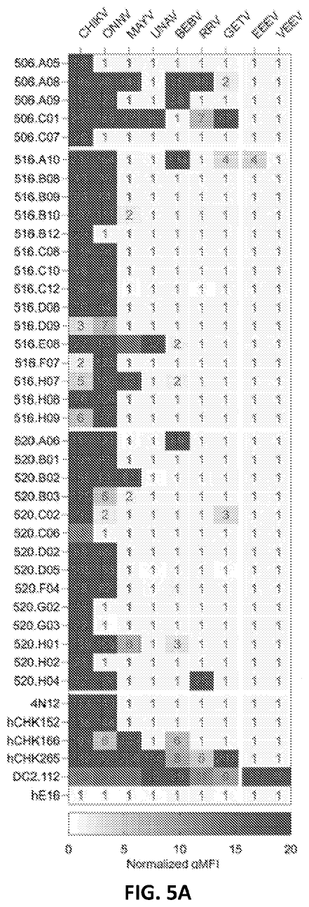

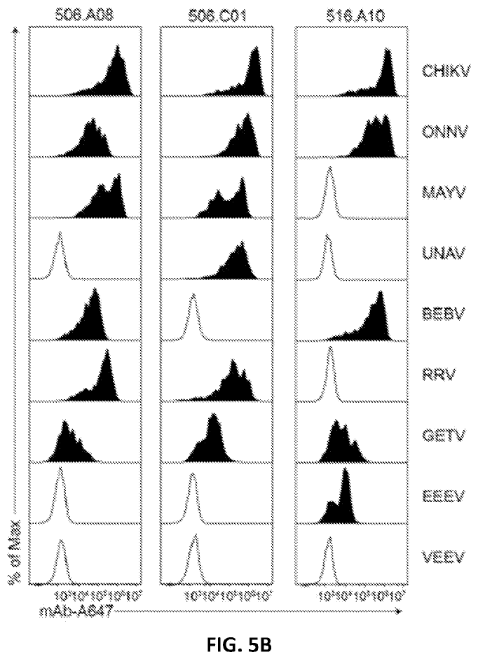

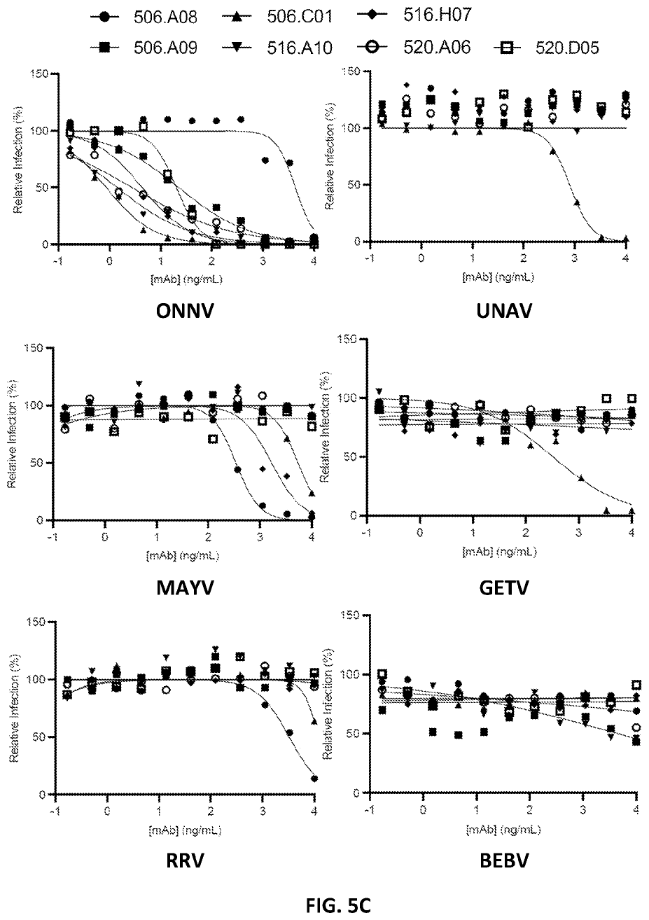

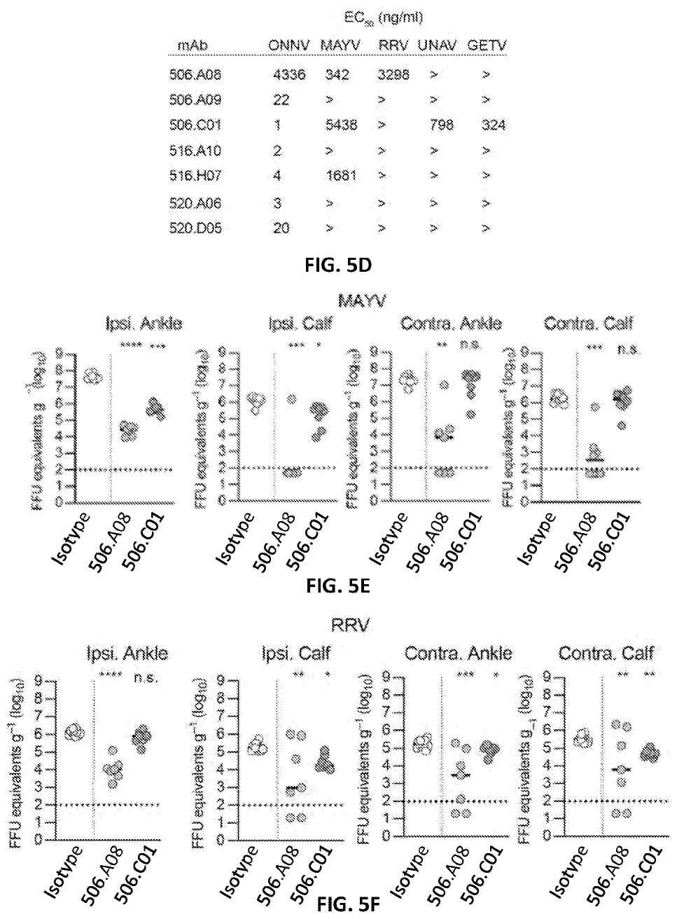

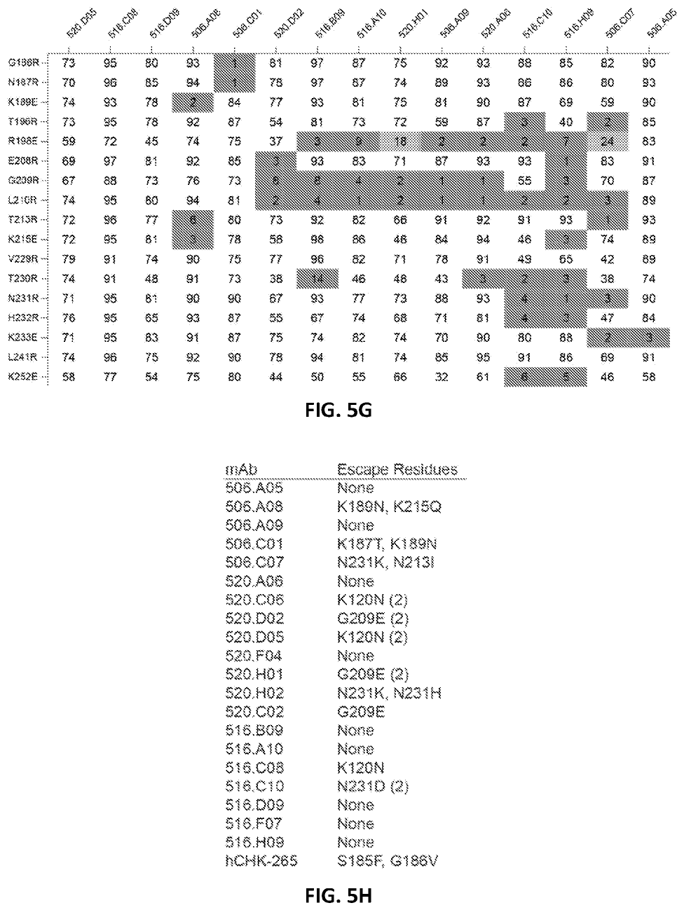

In some embodiments, binding to A domain and different areas of E2 domain are obtained by alanine-scanning mutagenesis, e.g., as shown in FIG. 4A. In some embodiments, the alanine-scanning mutagenesis results were further confirmed by charge reversal substitutions as shown in FIG. 5G. In some embodiments, the binding of the antibody 516.A10 is mapped to R198, G209 and L210, and the antibody 506.A5 is mapped to the residue K233 of the E2 glycoprotein using the charge-reversal mutations. In some embodiments, epitope mapping of an antigen binding molecule is performed by neutralization escape, e.g., as shown in FIG. 5H. The result is typically substantially consistent with that from alanine/arginine mutagenesis.

In some embodiments, close contacts between antibodies and CHIKV E2-Fab fragment were analyzed by cryo-EM model. Particularly, antibody 506.A08 has close contacts, through its residues S56, T57, T94, E105, E105, S102 and G103, to residues N193, G194, T213, D214, K215, V216 and N218 of the CHIKV E2 glycoprotein; and antibody 506.C01 has close contacts, through its residues D32, Y35, Y52, F54, T59, Y60, A91, N92, F94, Y104, C105 and K112, to residues Q184, S185, G186, K189, D214, V216, N218 and N219 of the CHIKV E2 glycoprotein.

In certain aspects, the present disclosure provides a pharmaceutical composition comprising the antigen binding molecule described above, e.g., a composition suitable for therapeutic use in a human.

In certain aspects, the present disclosure provides a method of preventing or treating a viral infection in a subject in need thereof, comprising administering to the subject a therapeutically effective amount of the antigen binding molecule(s) described herein or the pharmaceutical composition(s) described above.

In some embodiments, the viral infection is caused by an alphavirus. In some embodiments, the alphavirus is Chikungunya virus (CHIKV), O'nyong'nyong virus (ONNV), Mayaro virus (MAYV), Ross River virus (RRV), Una virus (UNAV), Bebaru virus (BEBV), Getah virus (GETV), Eastern equine encephalitis (EEEV), Venezuelan equine encephalitis (VEEV), Western equine encephalitis (WEEV), or a combination thereof.

### EXAMPLES

### Example 1. Methods Used in the Examples

Human cohort and sample collection. Human samples were obtained from Group 9 of NCT03483961, a Phase 2 trial evaluating the immune response to and safety profile of various doses and schedules of administration of PXVX0317 in healthy adults. Trial design and outcome are summarized in Bennett, S. R. et al, Safety and immunogenicity of PXVX0317, a purified aluminium hydroxide-adjuvanted chikungunya virus-like particle vaccine: a randomised, double-blind, parallel-group, phase 2 trial, Lancet Infect. Dis., 2022, doi:10.1016/S1473-3099(22)00226-2 (Bennett et al, 2022), which is incorporated herein by reference in its entirety. The participants in group 9 received two doses of 20 μg of PXVX0317 (alum-adjuvanted CHIKV VLP) at Day 1 and Day 28. Peripheral blood was collected by venipuncture into serum separator tubes, followed by centrifugation, followed by freezing of serum (supernatant) at −80° C. Peripheral blood mononuclear cells were isolated from anticoagulated leukopak collected in 50 mL conical tubes by density-gradient centrifugation (Ficoll) and frozen in liquid nitrogen after addition of 10% (final concentration) DMSO and 90% FBS. Monoclonal antibodies were generated from peripheral blood B cells of three PXVX0317-vaccinees on day 57 post-immunization. Single-cell RNA sequencing of bulk CD19⁺IgD-B cells from blood at Day 57 were performed. To identify VLP-specific cells, oligonucleotide-conjugated probes (streptavidin-TotalSeqC to label biotinylated-CHK-265) and B cell receptor sequencing, also termed LIBRA-Seq were used. Approximately 30 to 200 of the recovered B cells from each vaccinee bound to VLPs and were mapped almost exclusively to non-activated B cells (CD71^{lo}).

Expression of recombinant mAbs. The V-D-J region for each was codon-optimized, synthesized, and cloned into a mammalian expression vector (GenBank FJ475055 and FJ475056) containing a CMV promoter and the human IgG1 constant regions (Genscript). For antibody expression, plasmids (1:1 ratio of heavy and light chain vectors) were diluted into Opti-MEM, complexed with EXPIFECTAMINE transfection reagent (Thermo Fisher), and introduced into Expi293 cells (Thermo Fisher) in deep-well 96-well plates using (for micro-scale production) and 250 mL to 1000 mL baffled flasks (for midi-scale production) according to the manufacturer's recommendations.

For micro-scale purification, supernatant was harvested 5 days post-transfection, centrifuged to remove cellular debris, and incubated with MAGNE Protein G beads (Promega) for 4 hours at room temperature with shaking. Antibodies were purified with a Kingfisher instrument (Thermo Fisher) by sequential washing with PBS and elution with 0.2 M citric acid pH 3.0 followed by neutralization with 2 M Tris pH 7.4 and immediate buffer exchange into PBS with a 96-well Zeba desalting plate (Thermo Fisher). Antibody concentrations were determined with NANOORANGE Protein Quantitation kit (Thermo Fisher) according to the manufacturer's instructions and interpolated from a human IgG1 standard curve.

For midi-scale antibody purification (for in vivo studies), supernatants (50 to 200 mL) were harvested 5 to 6 days post-transfection, centrifuged to remove cell debris, and purified using Protein A Sepharose 4B (Thermo Fisher) with the same washing, elution and neutralization conditions as described above. Antibodies were desalted into PBS with PD-10 columns (Cytiva). Protein concentration was assessed by Nanodrop (Thermo Fisher), and purity was confirmed using SDS-PAGE analysis.

Virus and cell culture. Vero cells (CCL-81) were obtained from the American Type Culture Collection (ATCC) and maintained at 37° C. in Dulbecco's Modified Eagle medium (DMEM) supplemented with 10% fetal bovine serum (HYCLONE), 100 U/mL of penicillin, 100 μg/mL of streptomycin, 10 mM N-2-hydroxyethylpiperazine-N′-2-ethanesulfonic acid (HEPES), non-essential amino acids, and GlutaMAX (Thermo Fisher). The following alphaviruses were obtained from the World Reference Center for Emerging Viruses and Arboviruses and propagated and titered in Vero CCL-81 cells: CHIKV (AF15561, LR 2006, 37997, RSU1, and 181/25), ONNV (MP30), MAYV (BeH407), UNAV (BeAr 2380), RRV (T48), GETV (AMM 2021), and BEBV (MM 2354). SINV-EEEV (FL93-939) SINV-VEEV (TrD) have been described previously by Kim, A. S. et al, Protective antibodies against Eastern equine encephalitis virus bind to epitopes in domains A and B of the E2 glycoprotein, Nat Microbiol, 4: 187-197, 2019, and Sun, C., et al., Stable, high-level expression of reporter proteins from improved alphavirus expression vectors to track replication and dissemination during encephalitic and arthritogenic disease, J. Virol., 88:2035-2046, 2014.

Mouse experiments. Virus inoculations were performed under anesthesia that was induced and maintained with isoflurane, and all efforts were made to minimize animal pain and discomfort. 3-week-old male C57BL/6J mice were purchased from Jackson Laboratories (catalog #00664). For the lethal challenge model of CHIKV infection, 4-5 week-old male mice were treated with 500 μg of anti-IFNAR1 (MAR1-5A3, Leinco) by intraperitoneal injection and either 100 μg (˜5 mg/kg) or 20 μg (˜1 mg/kg) of the indicated mAb one day prior to subcutaneous inoculation of the rear footpad with 101 FFU of CHIKV-LR 2006. For immunocompetent models of alphavirus infection, 3- to 4-week-old male mice were administered 200 μg (˜10 mg/kg) of the indicated mAb one day prior to inoculation in the rear footpad with 10³ FFU of CHIKV-LR 2006, MAYV (strain BeH407), or RRV (strain T48). Foot swelling in this model was determined using digital calipers (Fowler) by measurement of the height and width of feet.

Viral burden analysis. At 3 days post infection, mice were euthanized, and tissues were collected. Tissues were homogenized with a Mag NA Lyser (Roche), and viral RNA was extracted with a MagMAX Viral RNA extraction kit using a Kingfisher Flex instrument (Thermo Fisher) according to the manufacturer's instructions. Viral load was determined using a TaqMan RNA-to-Ct 1-Step Kit on a QUANTSTUDIO 6 (Thermo Fisher) using previously established CHIKV-LR 2006, MAYV, and RRV primer and probe sets (Pinna, D., et al., “Clonal dissection of the human memory B-cell repertoire following infection and vaccination,” Eur. J. Immunol. 39, 1260 1270 (2009), Morrison, T. E., et al., A mouse model of chikungunya virus-induced musculoskeletal inflammatory disease: evidence of arthritis, tenosynovitis, myositis, and persistence, Am. J. Pathol., 178: 32-40, 2011, Kim, A. S., et al., Protective antibodies against Eastern equine encephalitis virus bind to epitopes in domains A and B of the E2 glycoprotein, Nat Microbiol, 4: 187-197, 2019). FFU equivalents were determined by parallel processing of a viral stock with known titer.

Virus-like particle production. Chikungunya VLPs of the 37997 strain were prepared. Briefly, suspension-adapted, human embryonic kidney 293 cells were transfected in serum free media with an expression plasmid containing the structural genes. Following centrifugation and filtration of the transfected harvest, VLPs were purified by tangential flow filtration followed by anion exchange chromatography. The purified VLPs were concentrated and buffer exchanged into 10 mM potassium phosphate, 218 mM sucrose, and 25 mM sodium citrate, sterile filtered and stored at −80° C. prior to use.

Recombinant Proteins. Recombinant mammalian cells expressing CHIKV E2 and E1 were purchased from Sino Biologicals (40440-V08B) or Native Antigen (CHIKV-E1-100), respectively. CHIKV p62-E1 (Voss, J. E., et al., Glycoprotein organization of Chikungunya virus particles revealed by X-ray crystallography, Nature, 468: 709-712, 2010) was expressed transiently in Expi293 cells according to the manufacturer's instructions with Expifectamine 293. Four days after transfection, supernatants were harvested, centrifuged, filtered, and concentrated and dialyzed overnight into 20 mM Tris pH 8.0, 200 mM NaCl using a Millipore Ultra-Cel 30 kDa membrane. Dialyzed supernatant was passed over Ni-NTA agarose column (Thermo Fisher), and purified protein was eluted with 250 mM imidazole (pH 8.0). Eluted protein was exchanged into 1×PBS with PD-10 columns followed by concentration with Amicon centrifugal filter with a 30 kDa cutoff. Protein purity was assessed by SDS-PAGE, and proteins were stored frozen at −80° C.

ELISA. (a) VLPs. MAXISORP plates (Thermo Fisher) were coated with 1 μg/mL of alphavirus cross-reactive mAbs with MAY-117 and MAY-119 (Earnest J T, et al., Neutralizing antibodies against Mayaro virus require Fc effector functions for protective activity, J Exp Med. 216(10):2282-230 (2019)) overnight in 0.1 M NaHCO₃ buffer pH 9.3 at 4° C. After washing, CHIKV VLPs were added in blocking buffer (2% BSA in PBS) at a concentration of 1 μg/mL. (b) E2 and p62-E1. For CHIKV E2 (Sino Biological) and CHIKV p62-E1 ELISA, plates were coated with 2 μg/mL of purified recombinant protein. MAbs were diluted in blocking buffer and added to plates for 1 hour at 25° C. Plates were washed and incubated with horseradish peroxidase conjugated goat-anti human IgG (H+L) (1:10,000 dilution, Jackson ImmunoResearch) for 30 min at 25° C. After washing, plates were developed with 1-Step™ Ultra TMB-ELISA substrate (Thermo Fisher), stopped with 2 N H2SO₄, and read at 450 nM using a Synergy H1 plate reader or Cytation 7 (Biotek).

For epitope binning experiments, plates were coated with CHIKV p62-E1 as described above followed by incubation with 20 μg/mL of each mAb for 1 hour at room-temperature. Biotinylated mAbs then were directly applied without washing at a concentration of 50 ng/mL and incubated for 30 min at room temperature. Following incubation, plates were washed and incubated with streptavidin-HRP (Jackson, 1:10,000) for 20 min at room temperature and ultimately developed with 1-Step™ Ultra-TMB, as described above.

Focus reduction neutralizing test. Serially diluted mAbs or sera were incubated with ˜200 FFU of different alphaviruses for 1 hour at 37° C. The antibody-virus complex then was added to Vero cells for 60 min at 37° C. followed by a 1% methylcellulose overlay in Minimal Essential Medium supplemented with 2% FBS (HyClone). Cells were fixed at 16 hours post-infection with 1% paraformaldehyde (PFA; Electron Microscopy Sciences) in PBS. Cells were washed with PBS-T (0.05% Tween-20) and incubated for 2-4 hours at room-temperature or overnight at 4° C. with 1 μg/mL of CHK-48, CHK-265, or DC2.112 (Pal, P. et al., “Development of a highly protective combination monoclonal antibody therapy against Chikungunya virus,” PLOS Pathog., 9, e1003312 (2013), Quiroz, J. A. et al., Human monoclonal antibodies against chikungunya virus target multiple distinct epitopes in the E1 and E2 glycoproteins, PLOS Pathog., 15: e1008061, 2019, Kim, A. S. et al., Pan-protective anti-alphavirus human antibodies target a conserved E1 protein epitope, Cell, 184:4414-4429.e19, 2021). After washing, cells were incubated for 1 hour with HRP-conjugated goat anti-mouse or goat anti-human IgG (H+L) (1:2,000; Jackson ImmunoResearch). Plates were developed using TrueBlue substrate (KPL), foci were quantitated using a BioSpot plate reader, and neutralization data was analyzed with Prism 9 (GraphPad).

Flow cytometry of blood samples. Samples were thawed rapidly at 37° C. and stained with CHIKV VLPs at a concentration of 10 μg/mL in FACS buffer (0.1% BSA+0.05% NaN₃⁺2 mM EDTA in 1×PBS) for 30 min at 4° C. Cells were subsequently stained with the following monoclonal antibodies and reagents from BioLegend: BV785-conjugated anti-CD19 (HIB19), BV421-conjugated anti-IgD (IA6-2), APC-Cy7-conjugated anti-CD38 (HIT-2), PE-Cy7-conjugated anti-CD71 (RI7217), BV605-conjugated anti-CD27 (O323), PE-Dazzle594-conjugated anti-CD4 (A161A1). CHK-265 was conjugated to Alexa Fluor 488 and Alexa Fluor 647 with a Zip Antibody Labelling Kit (Thermo Fisher) according to the manufacturer's instructions. Samples were analyzed in the presence of DAPI (0.1 μg/mL) to exclude dead cells on a Cytek Aurora 3-laser (VBR) instrument. Data were analyzed with FloJo software version 10 (Treestar).

Single-cell RNA sequencing. Approximately 2 to 3×10⁶ PBMCs were thawed and incubated sequentially with 10 μg/mL of CHIKV VLP for 30 min on ice and 1 μg/mL of biotinylated CHK-265. Cells then were stained with the following cocktail of reagents purchased from Biolegend unless otherwise noted: streptavidin-APC-TotalSeqC (Cat #405293), anti-CD19-TotalSeqC (Cat #302265), anti-CD71-TotalSeqC (Cat #334125) and anti-CD27-TotalSeqC (Cat #302853), PE-conjugated anti-IgD (IA6-2) and PerCP-Cy5.5-conjugated anti-CD20 (2H7). Naïve B cells were depleted with anti-PE Mojo magnetic nanobeads, and B cells were enriched using EASYSTEP™ pan B cell magnetic enrichment kit (STEMCELL). Approximately 20,000 to 30,000 non-naïve enriched B cells were obtained for downstream 10× Genomics analysis using this protocol.

VDJ, 5′ gene expression, and probe feature libraries (TotalSeq-C) were prepared using the 10× Chromium System (10× Genomics). The Chromium Single Cell 5′ Library and Gel Bead v2 Kit, Human B Cell V (D) J Enrichment Kit, and Feature Barcode Library Kit were used. Libraries were pooled and sequenced using the NovaSeq (Illumina) 6000. Raw sequencing reads were processed using standard Cell Ranger (version 3.0.2) pipeline, including 5′ gene expression analysis, antigen probe analysis, and immunoprofiling analysis of B cells.

Cell Ranger output was processed using Seurat (an R package, for transcriptome, cell surface protein and antigen probe analysis). For transcriptome analysis, Seurat was used for cell quality control, data normalization, data scaling, dimension reduction (both linear and non-linear), clustering, and data visualization. Unwanted cells were removed according to the number of detectable genes (number of genes <200 or >2,500 were removed) and percentage of mitochondrial genes for each cell. Transcriptome data were normalized by a log-transform function with a scaling factor of 10,000, whereas cell surface proteins and antigen probes were normalized by a centered log-ratio (CLR) normalization. All computational analyses were performed in R.

Antibody binding to alphavirus-infected cells. Vero cells were inoculated (multiplicity of infection [MOI] of 5) with different alphaviruses in DMEM supplemented with 2% FBS. After allowing infection to proceed for 14 to 18 hours, cells were detached using TrypLE (Thermo Fisher), washed with DMEM containing 2% FBS, and filtered through 100 μm nylon mesh (Corning). Cells then were incubated with 10 g/mL of mAbs for 30 min at 4° C. in FACS buffer. Cells were washed and incubated with Alexa Fluor 647 conjugated goat anti-human or anti-mouse IgG (1:2,000 dilution; Thermo Fisher) for 30 min at 4° C. Cells were washed, fixed with 2% paraformaldehyde (PFA), and subjected to flow cytometry analysis using an iQue3 flow cytometer (Sartorius).

Alanine-scanning and charge-reversal mutagenesis. A pCAGGS plasmid encoding CHIKV VLP (capsid-E3-E2-6K-E1) was subjected to mutagenesis of all residues of CHIKV E2 to alanine except for alanine residues, which were mutated to serine (Genscript). Select residues identified by alanine-scanning were separately mutated to arginine with arginine and lysine residues mutated to glutamate (Genscript). Plasmids were transfected in a 96-well format with Expifectamine 293 into Expi293 cells (Thermo Fisher). 24 hours post-transfection, cells were stained with pre-titrated sub-saturating concentrations of mAbs for 30 min at 4° C. followed by secondary staining with anti-human IgG Alexa Flour 647 for 20 min at 4° C. Cells were resuspended in FACS buffer containing DAPI for exclusion of dead cells. Data were acquired on an iQue3 flow cytometer, and analyzed with ForeCyt software (Sartorius).

Epitope binning by flow cytometry. CHIKV structural proteins (capsid-E3-E2-6k-E1) were expressed on the surface of Expi293 cells by transfection as described above. Cells were harvested 24 hours after transfection and half were labelled with CellTrace™ CFSE (Thermo Fisher) according to the manufacturer's recommendations. Labelled cells were then pre-incubated with mouse mAbs or MXRA8-mouse Fc at a concentration of 10 μg/mL for 1 hour at 4° C. Cells were extensively washed with FACS buffer and combined in a 1:1 ratio with CFSE-negative cells. The cell mixture was then incubated with mAbs at a concentration of 1 μg/mL for 30 min at 4° C., followed by incubation with anti-human IgG-A647 (Thermo Fisher) for 20 min at 4° C. Cells were washed, and resuspended in FACS buffer supplemented with DAPI and analyzed on an iQue3 flow cytometer. Percent binding was calculated for each mAb by geometric MFI in CFSE⁺ divided by CFSE⁻ cells in each sample multiplied by 100.

Biolayer interferometry. BLI experiments were performed using an Octet Red96 (ForteBio) at 25° C. in 10 mM HEPES, pH 7.4, 150 mM NaCl, 3 mM EDTA, 0.005% P20 surfactant, and 1% BSA (w/v). MAbs were immobilized onto anti-human IgG Fc capture biosensors (ForteBio) and dipped into wells containing 1 μM CHIKV E1 (Native Antigen) to assess E1 reactivity of mAbs.

Neutralization escape. To generate neutralization escape mutants, CHIKV 181/25 (10⁵ FFU) was incubated with 10 μg/mL of anti-CHIKV mAbs for 1 hour at 37° C. The virus-mAb complexes were added to Vero cells. One day post-infection, the virus supernatant was removed and then incubated with mAbs for 1 hour at 37° C. and added to new Vero cells. This process was repeated for 5 days. Viral RNA from bulk supernatant was extracted with MagMAX Viral RNA extraction kit using a Kingfisher Flex instrument (Thermo Fisher) according to the manufacturer's instructions. cDNA was generated using ProtoScript® II First Strand cDNA Synthesis Kit (NEB) according to the manufacturer's instructions using random hexamers. Viral structural genes were amplified with the following primers: 5′-TGCCATTCCAGTTATGTGCC-3′ (SEQ ID NO:305) and 5′-CACGCATAGCACCACGATTA-3′ (SEQ ID NO:306), purified with ChargeSwitch PCR clean-up kit (Thermo Fisher) and subject to long-read amplicon sequencing, Oxford Nanopore (Plasmidsaurus).

Cryo-EM sample preparation. Fab was generated from mAbs 516.A08, 516.C01, and chimeric human CHK-265 using a FabALACTICA Fab kit (Genovis) according to manufacturer's instructions. CHIKV VLPs were prepared at a nominal concentration of ˜0.7 mg/mL in 10 mM potassium phosphate, 218 mM sucrose, and 25 mM sodium citrate. VLPs were incubated for 1 hour on ice with approximately 2:1 molar excess of each Fab, applied to glow-discharged lacey carbon grids (Ted Pella #01895-F), and then flash-frozen in liquid ethane using a Vitrobot Mark IV (Thermo Fisher).

Cryo-EM data collection. Grids were loaded into a Cs-corrected FEI Titan Krios 300 kV microscope equipped with a Falcon 4 direct electron detector and then imaged at a nominal magnification of 59,000×, resulting in a calibrated pixel size of 1.16 Å. Movies were collected in EER format with a total dose of 38.25 e⁻/Å²/movie (4.70 e⁻/Å²/s over 8.14 s acquisition).

Cryo-EM data processing. EER movies were binned into 35 fractions (1.09 e⁻/Å²/f) and then subjected to patch motion and patch CTF corrections in cryoSPARC v3.1.0. Particles were selected using a template picker then cleaned via two- and three-dimensional classification. Whole VLPs were reconstructed via homogeneous non-uniform refinement with I1 symmetry imposed. To extract asymmetric units as individual subparticles, the method performed I1 symmetry expansion, applied particle shifts to center asymmetric units, and then re-extracted these particles with shifts applied. These asymmetric units were cleaned via three-dimensional classification without alignment in Relion 3.1, and the class of highest resolution for each Fab was refined via local non-uniform refinement in cryoSPARC.

Model building. To facilitate model building, maps were sharpened using deep learning implemented via DeepEMhancer. Starting models for CHIKV structural proteins were adapted from a previous CHIKV VLP cryo-EM structure (PDB: 6NK5), and initial Fv were modeled using AlphaFold2 implemented in ColabFold. These starting components were docked into the electron density maps and then refined iteratively using Coot v0.9.5, Isolde v1.1.0, Phenix v1.19, and Rosetta. Proteins, Interfaces, Structures, and Assemblies (PISA) solvent exclusion analysis was used to identify contact residues and calculate buried surface area. Structures were visualized using UCSF ChimeraX.

Phylogenetic inference. E2 sequences were retrieved from the NCBI GenBank for each alphavirus: CHIKV (QKY67868.1), MAYV (QED21311.1), Una (UNAV, YP_009665989.1), ONNV (AAC97205.1), SFV (NP_463458.1), RRV (AAA47404.1), EEEV (ANB41743.1), VEEV (AGE98294.2), SINV (AAM10630.1), WEEV (QEX51909.1), GETV (ABK32032.1), and BEBV (AEJ36225.1). Sequences were aligned via Clustal Omega with simple phylogeny inferred from pairwise distances. Results were visualized in R using the ggtree package.

Statistical analysis. Statistical analysis was performed with Prism 9.0. Details of statistical tests are included in figure legends and include Kruskal Wallis with Dunn's post-test correction for virological analysis and Kaplan-Meier analysis with Bonferroni correction for survival studies.

Data availability. Models of mAb complexes were generated from their respective PDB files of CHK-265 (PDB: 6W09), 506.A08, and 506.C01. The generated structures are deposited as PDB 8DWY (Chikungunya VLP in complex with neutralizing Fab CHK-265 (asymmetric unit)), 8DWW (Chikungunya VLP in complex with neutralizing Fab 506.A08 (asymmetric unit)), and 8DWX (Chikungunya VLP in complex with neutralizing Fab 506.C01 (asymmetric unit)) in RCSB Protein Data Bank on May 31, 2023.

### Example 2. Functional Properties of mAbs

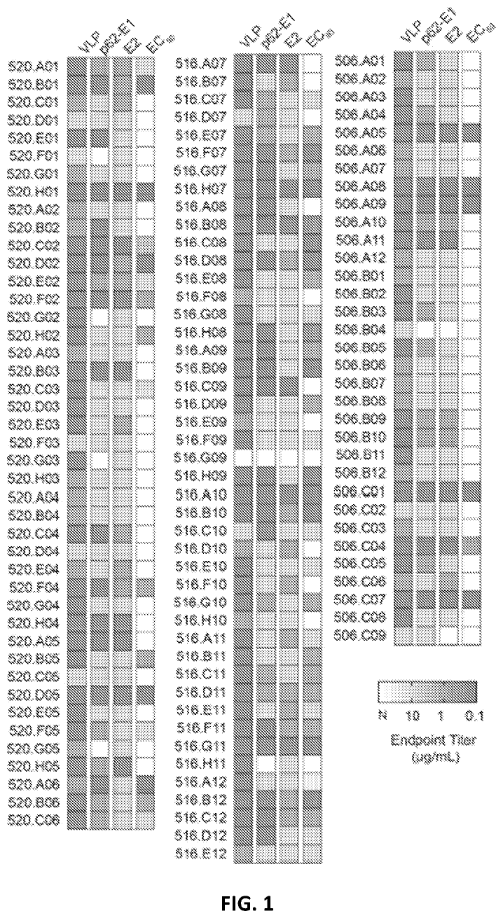

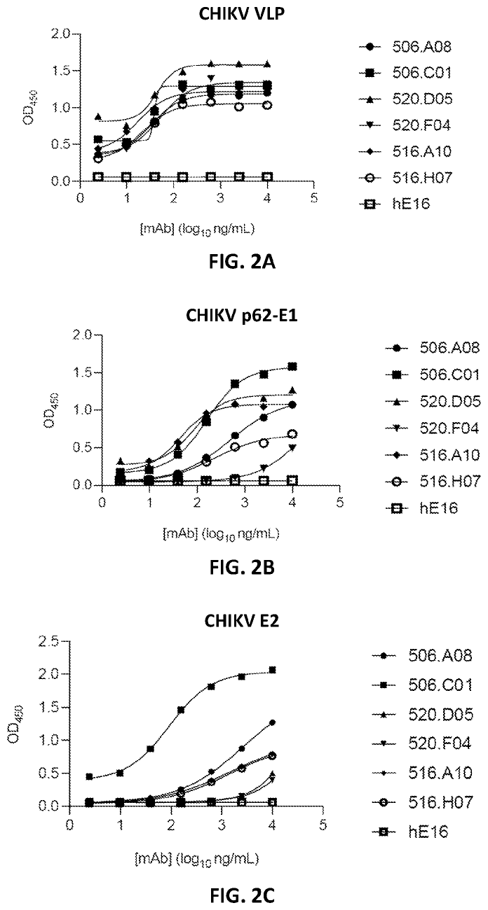

As shown in FIG. 1, nearly all expressed mAbs bound to VLPs, and many bound at concentrations below 100 ng/mL, validating the specificity of the approach. Binding to the constituent E2-E1 heterodimer proteins were also tested by ELISA using recombinant p62-E1 protein derived from the CHIKV-LR 2006 strain. Although the majority of VLP-binding mAbs bound p62-E1, a few (e.g., 520.F01 and 520.G03) did not bind even at 10 μg/mL, potentially indicating engagement of quaternary epitopes or a genotype-dependent (5% amino acid difference in E2-E1 compared to strain CHIKV 37997) loss in p62-E1 binding. Moreover, most of the VLP-binding antibodies also bound to soluble recombinant E2 by ELISA (FIGS. 1, 2A and 2B). Note that some mAbs (520.G02, 520.G03, 520.G05, 516.H11) bound E2 but not p62-E1 appreciably. However, these mAbs generally bound weakly, and this finding could reflect differential limits of detection with the ELISA-based assays.

mAbs were screened for inhibitory activity by performing FRNTs at selected (100 ng/mL, 1 μg/mL, and 10 μg/mL) concentrations against the CHIKV-LR 2006 strain. Notably, 54 of 121 mAbs neutralized CHIKV-LR 2006 infection at the highest tested concentration (10 μg/mL), and 20 of 121 mAbs inhibited infection at concentrations below 100 ng/ml (FIG. 1).

### Example 3. Neutralization and Epitope Binning of mAbs

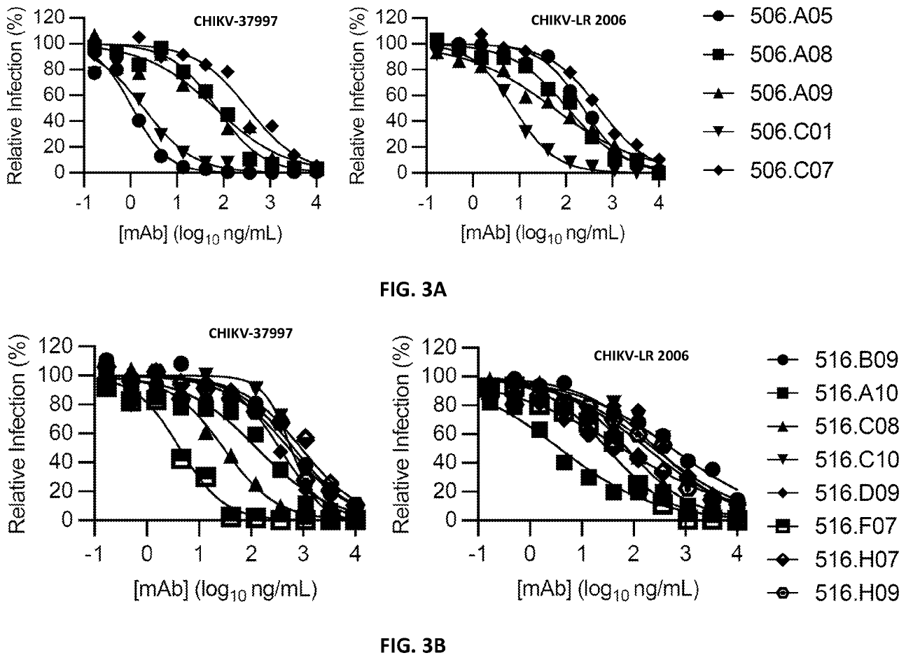

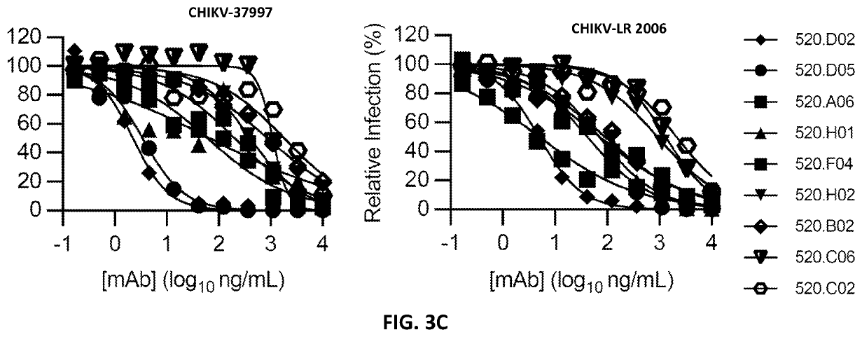

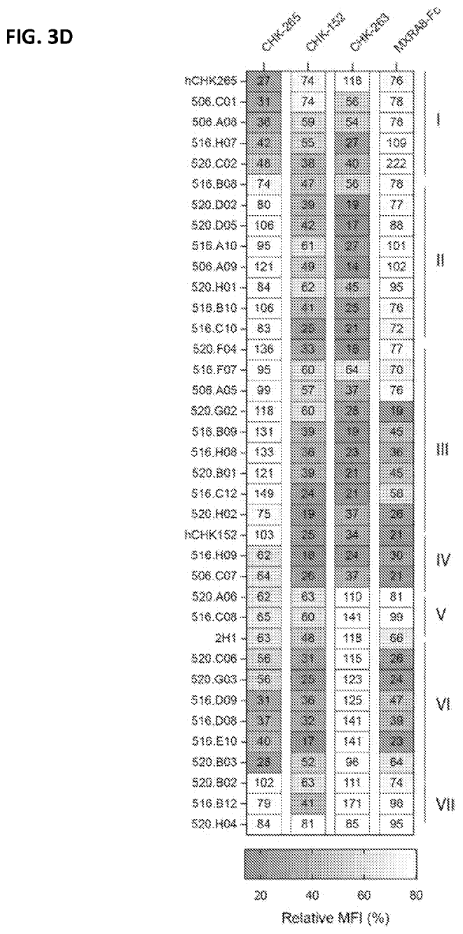

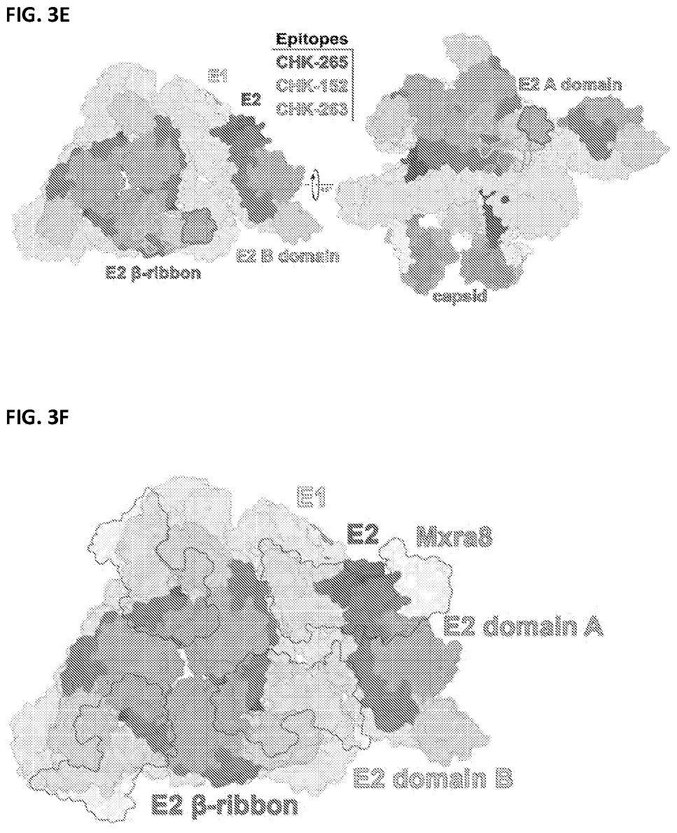

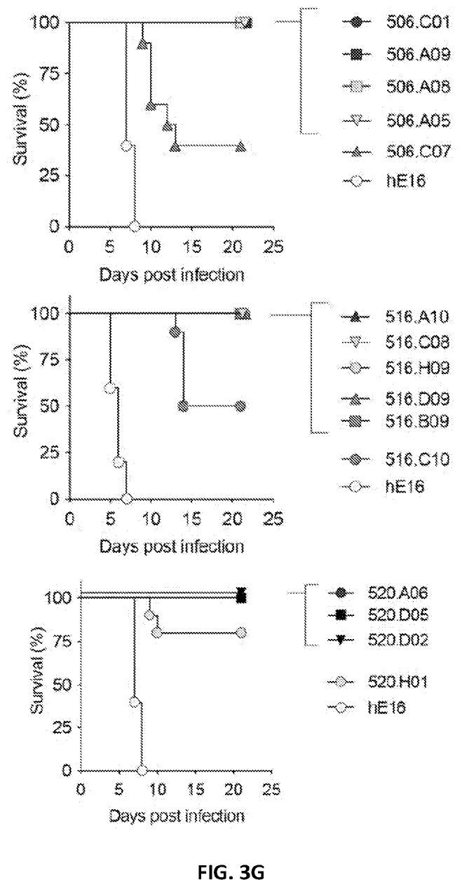

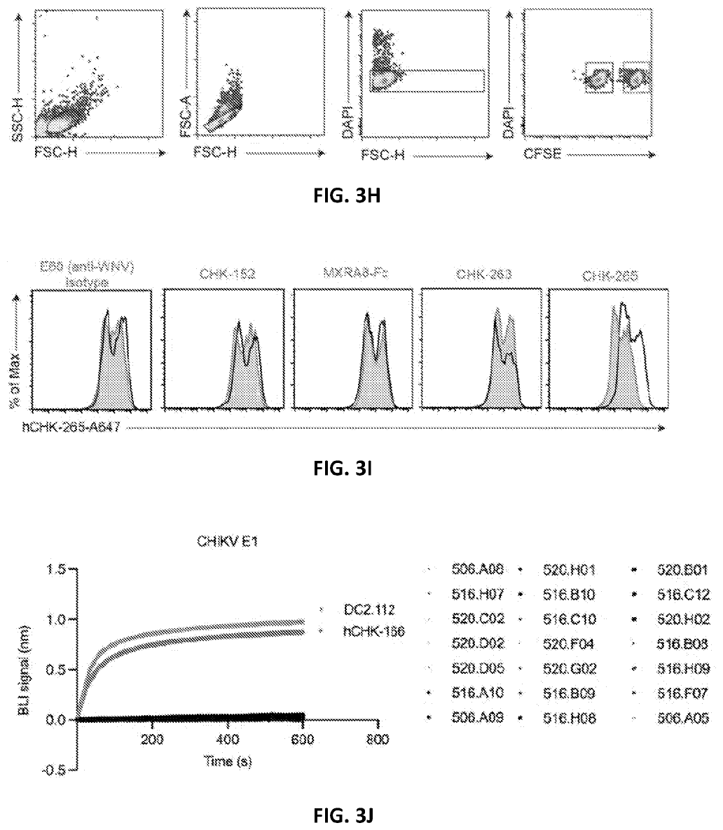

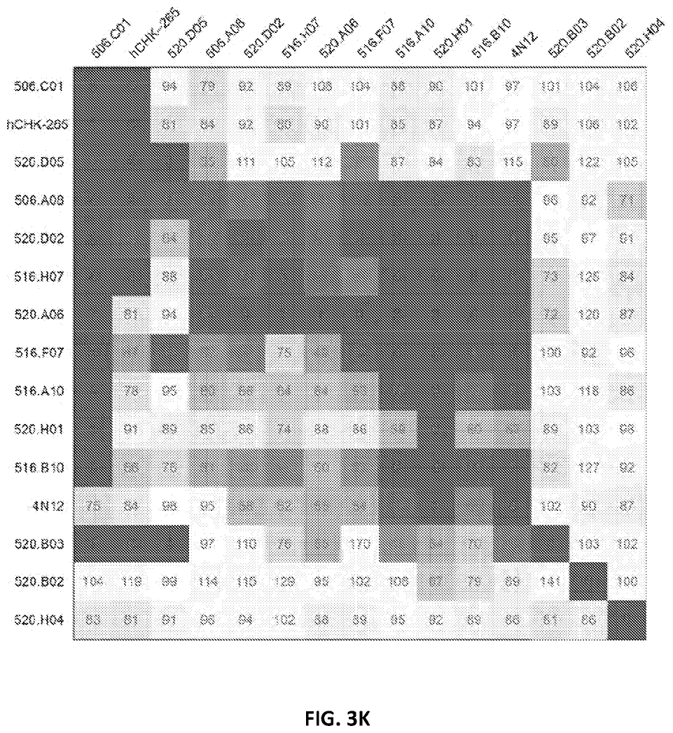

To assess the neutralizing potency of the mAbs more quantitatively, full dose-response FRNTs were performed against the immunizing strain CHIKV-37997 and CHIKV-LR 2006 for the subset of mAbs that were the most potently inhibitory in the initial screen shown in FIG. 1. Notably, as shown in FIGS. 3A-3C and Table 4, several mAbs showed strong inhibitory activity, with EC₅₀ values <100 ng/mL. Two mAbs (506.C01 and 520.D02) showed ‘elite’ inhibitory activity against both CHIKV strains, with EC₅₀ values <10 ng/ml.

TABLE 4
Table of EC50 Values of Indicated mAbs

| | 37997 | LR 2006 |
|---|---|---|
| | EC₅₀ | EC₅₀ |
| mAb | (ng ml⁻¹) | (ng ml⁻¹) |
| 506.C01 | 2 | 8 |
| 506.A09 | 67 | 55 |
| 506.A08 | 72 | 110 |
| 506.A05 | 1 | 236 |
| 506.C07 | 333 | 515 |
| 516.F07 | 4 | 31 |
| 516.C08 | 30 | 75 |
| 516.A10 | 128 | 4 |
| 516 H09 | 526 | 182 |
| 516.C10 | 582 | 279 |
| 516.D09 | 350 | 248 |
| 516.B09 | 655 | 555 |
| 516.H07 | 910 | 61 |
| 520.D02 | 2 | 5 |
| 520.D05 | 3 | 42 |
| 520.A06 | 60 | 5 |
| 520.H01 | 67 | 70 |
| 520.F04 | 202 | 72 |
| 520.H02 | 407 | 920 |
| 520.B02 | 870 | 90 |
| 520.C06 | 1096 | 1311 |
| 520.C02 | 1814 | 2116 |

To define the epitopes of the neutralizing human mAbs, competition-binding assays were performed with well-characterized reference mAbs derived from mice. A flow cytometry assay was developed with cells expressing the structural proteins (capsid-E3-E2-6K-E1) on the plasma membrane following transfection, which mimics envelope glycoprotein expression patterns after viral infection. First, as shown in FIG. 3D, cells with reference mouse mAbs: CHK-265 (E2, B domain), CHK-263 (E1, E2 B domain), and CHK-152 (E2, A/B domain) were stained (Sun, S. et al., Structural analyses at pseudo atomic resolution of Chikungunya virus and antibodies show mechanisms of neutralization, Elife, 2: e00435, 2013; Pal, P. et al., Development of a highly protective combination monoclonal antibody therapy against Chikungunya virus, PLOS Pathog, 9: e1003312, 2013; Malonis, R. J. et al., Near-germline human monoclonal antibodies neutralize and protect against multiple arthritogenic alphaviruses, Proc. Natl. Acad. Sci., 118(37):e2100104118, 2021). Competition binding with MXRA8, a mammalian cell surface receptor for CHIKV, was also performed using recombinant MXRA8-mouse Fc protein, as shown in FIG. 3D. After binding of the reference mAbs or MXRA8-Fc, transfected cells were incubated with mAbs from the panel followed by detection with A647-conjugated anti-human IgG (FIG. 3H and FIG. 3I). A majority of tested mAbs (20 of 34) competed with CHK-152 and CHK-263 but not with CHK-265, designated here in FIG. 3D as clusters II and III. Given that CHK-263 makes contacts with E130, binding of cluster II and III mAbs to purified E1 were tested. However, as shown in FIG. 3J, none demonstrated binding. Cluster I contained fewer mAbs (e.g., 506.A08, 506.C01, and 516.H07), which competed with CHK-265, a broadly-neutralizing E2 B domain mAb (FIG. 3D). Even though a majority of the mAbs in the competition panel were neutralizing, only a subset (clusters III and VI) competed substantively (>50% reduction) with MXRA8-Fc binding (FIG. 3D). Thus, based on competition-binding data, evaluated neutralizing mAbs appear to bind multiple antigenic sites on CHIKV E2, an important feature that decreases the likelihood of viral escape.

As an orthogonal method, a competition-binding assay to recombinant CHIKV p62-E1 in the solid-phase by ELISA were performed. The panel of mAbs were down-selected to those with the highest level of binding to p62-E1 protein. 15 mAbs were biotinylated and binding assays were performed with pre-bound non-biotinylated versions of each mAb. Previously characterized CHIKV-specific human mAbs (Smith, S. A. et al., Isolation and Characterization of Broad and Ultrapotent Human Monoclonal Antibodies with Therapeutic Activity against Chikungunya Virus, Cell Host Microbe, 18:86-95, 2015) were also included to provide context for the results. Consistent with the cell-based competition, as shown in FIG. 3K, mAbs in cluster I (506.C01 and 506.A08) competed with each other and with CHK-265. However, the majority of the other mAbs formed one large competition group that included antibodies recognizing epitopes in both the A and B domain. Notably, mAbs 520.B02 and 520.H04 did not compete with any other mAb, suggesting they engage unique epitopes.

### Example 4. Protection of mAbs In Vivo

mAbs from different competition clusters were tested for protective activity in vivo. A pathogenesis model in immunodeficient mice is used in which all animals succumb to CHIKV infection within one week (Pal, P. et al., Development of a highly protective combination monoclonal antibody therapy against Chikungunya virus, PLOS Pathog, 9: e1003312, 2013). A single 100 μg (˜5 mg/kg) dose of anti-CHIKV mAb as well as 500 μg (˜25 mg/kg) of anti-IFNAR1 mAb (MAR1-5A3) were administered one day prior to subcutaneous inoculation with 10 focus-forming units (FFU) of CHIKV-LR 2006. As shown in FIG. 3G, the most potently neutralizing mAbs completely protected mice against lethality, whereas animals administered an isotype control mAb (hE16, anti-West Nile virus (WNV)) succumbed to infection within 6 to 8 days of infection. The anti-CHIKV mAbs (520.H01, 506.C07, and 516.C10) with less neutralizing capacity demonstrated variable (40 to 80% survival) protection. For mAbs conferring complete protection at 10 mg/kg, experiments were repeated with lower doses to identify those with the greatest activity in vivo. As shown in FIGS. 4G-4I, at a 20 μg-dose (1 mg/kg), only 516.A10 conferred 100% protection. Most mAbs demonstrated >50% protection at this dose including: 520.D02, 520.D05, 506.A08, 506.A09, and 506.C01. In contrast, 506.A05 and 516.D09 lost protection, which correlated with their poorer neutralization potency.

Next, a subset of mAbs (506.A08, 506.A09, 506.C01, 516.B09, 516.D09, 516.C10, 520.D02, 520.D05, 520.A06) with varying neutralization activity were tested in an immunocompetent mouse model of CHIKV infection (Morrison T E, et al., A mouse model of chikungunya virus-induced musculoskeletal inflammatory disease: evidence of arthritis, tenosynovitis, myositis, and persistence, Am J Pathol. 178(1): 32-40 (2011)). C57BL/6 mice were inoculated subcutaneously in the rear footpad, which results in foot and ankle swelling, immune cell (monocyte and CD4⁺ T cell) infiltration, and viral burden in adjacent and distant musculoskeletal tissues. When administered as prophylaxis at a dose of 10 mg/kg, as shown in FIG. 4J, all anti-CHIKV mAbs reduced foot swelling compared to animals treated with an isotype-control mAb. Further, consistent with lack of clinical disease, as shown in FIGS. 4K-4N, viral burden in all analyzed tissues including ankle and calf showed substantial reductions by all tested anti-CHIKV mAbs compared to the isotype-control mAb.

### Example 5. Epitope Mapping by Alanine-Scanning Mutagenesis and Neutralization Escape

To better define the epitopes engaged by mAbs that demonstrated in vivo protection, alanine-scanning mutagenesis coupled with cell-surface display and high-throughput flow cytometry were performed to identify residues in the E2 glycoprotein required for mAb binding. Cells were transfected with capsid-E3-E2-6K-E1 expression plasmids encoding individual proteins with alanine (or serine for alanine) substitutions (residues 1-270) in the E2 gene (Akahata, W. et al., A virus-like particle vaccine for epidemic Chikungunya virus protects nonhuman primates against infection, Nat. Med., 16:334-338, 2010). Critical interaction residues were defined as those with <25% binding to a given mAb that retained >90% binding to an anti-CHIKV oligoclonal antibody mixture. As shown in FIGS. 4A and 4B, a majority of the inhibitory mAbs mapped to the B domain of E2. 506.C01 and 506.A08 mapped to the apex of the B domain (FIG. 4A and FIG. 4C), 520.D02 and 520.A06 mapped to the flank of the B domain (FIG. 4A and FIG. 4D), and 516.C10, 516.H09 and 506.C07 mapped to residues in both the B domain and the β-ribbon connecting the A and B domains (FIG. 4A and FIG. 4E). Residues in the A domain critical for binding of 516.D09, 516.C08, and 520.D05 mAbs were also identified. The alanine-scanning mutagenesis results were confirmed with charge reversal substitutions which resulted in even greater loss-of-binding than alanine mutations, as shown in FIG. 5G. Moreover, 516.A10 was mapped to R198/G209/L210 and 506.A05 was mapped to K233 by charge-reversal mutations, which did not meet criteria as critical residues by alanine scanning mutagenesis. Epitope mapping were also performed by neutralization escape by passaging CHIKV (strain 181/25) in the presence of 10 μg/mL of each mAb. Sequencing of the structural genes revealed mutations in the same or neighboring residues identified by alanine/arginine mutagenesis for all mAbs, which validated the alanine-scanning approach with an orthogonal method (FIG. 4A and FIG. 5H).

### Example 6. Reactivity of Protective mAbs Against Related Alphaviruses

To assess the cross-reactivity of the protective mAbs of the present disclosure against other alphaviruses, binding to the surface of virus-infected Vero cells were evaluated by flow cytometry and included additional mAbs from the panel of the disclosure as comparators. As shown in FIG. 5A, a majority of the mAbs cross-reacted with ONNV. A smaller number of mAbs cross-reacted with MAYV, and a few bound to cells infected with more distantly related alphaviruses including UNAV, RRV, or Bebaru virus (BEBV). As shown in FIGS. 5A and 5B, the neutralizing mAbs 506.C01 and 506.A08, both from cluster I, showed broad reactivity and bound to the cell surface of ONNV, MAYV, UNAV, GETV and RRV-infected cells, similar to the binding pattern of CHK-265. Further, one potently neutralizing mAb, 516.A10, showed cross-reactivity with ONNV, BEBV, and also GETV and EEEV, as shown using a chimeric Sindbis virus (SINV) expressing the EEEV structural proteins.

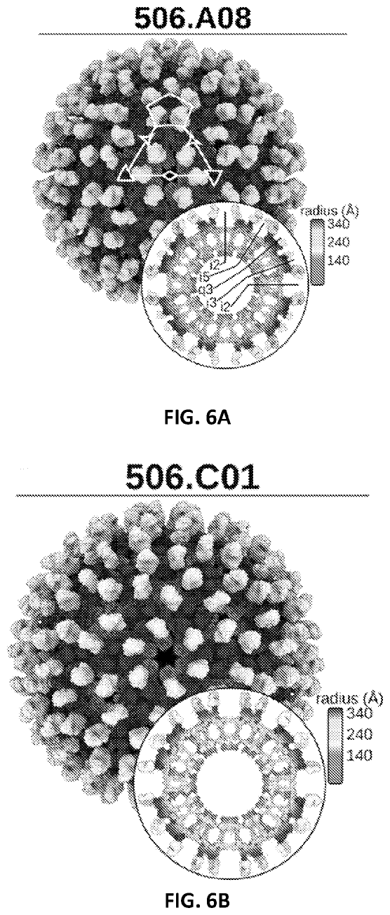

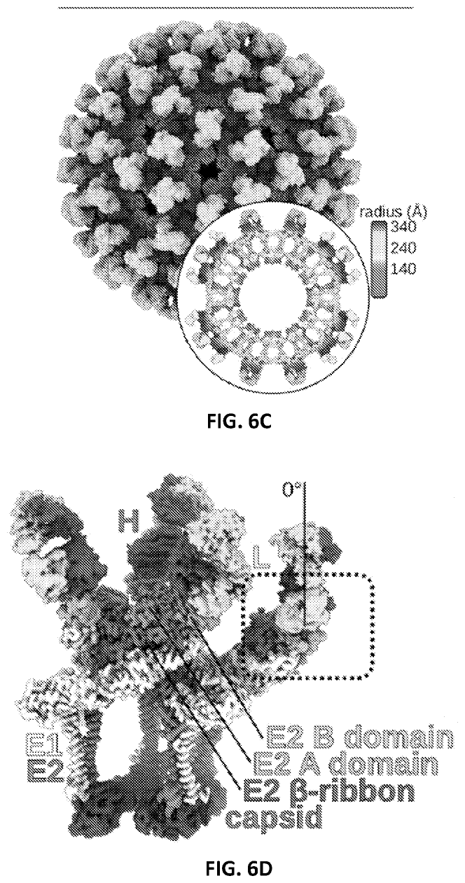

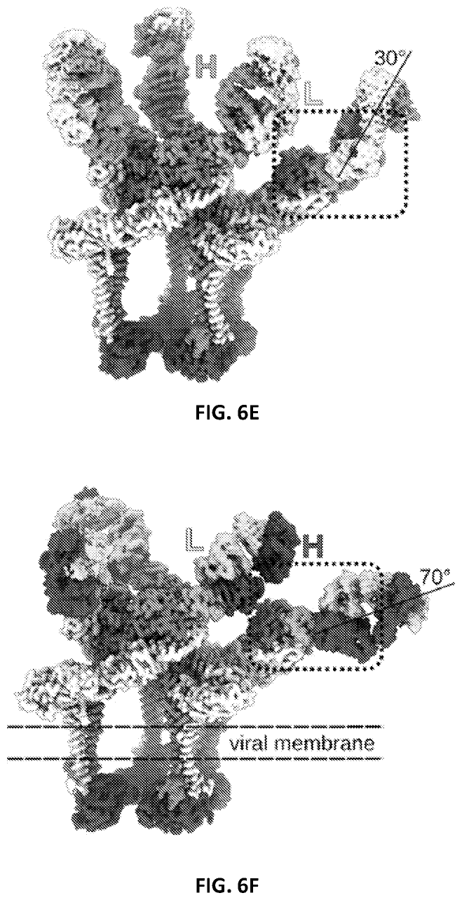

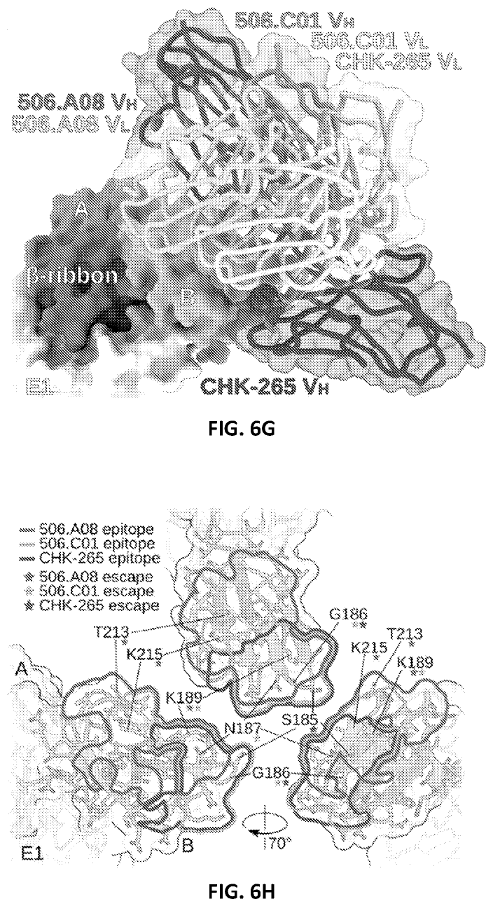

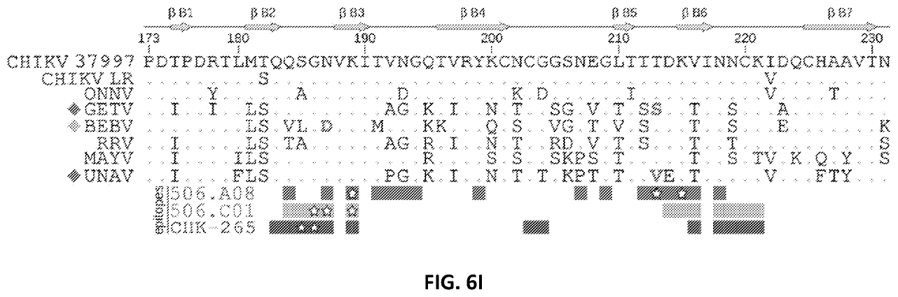

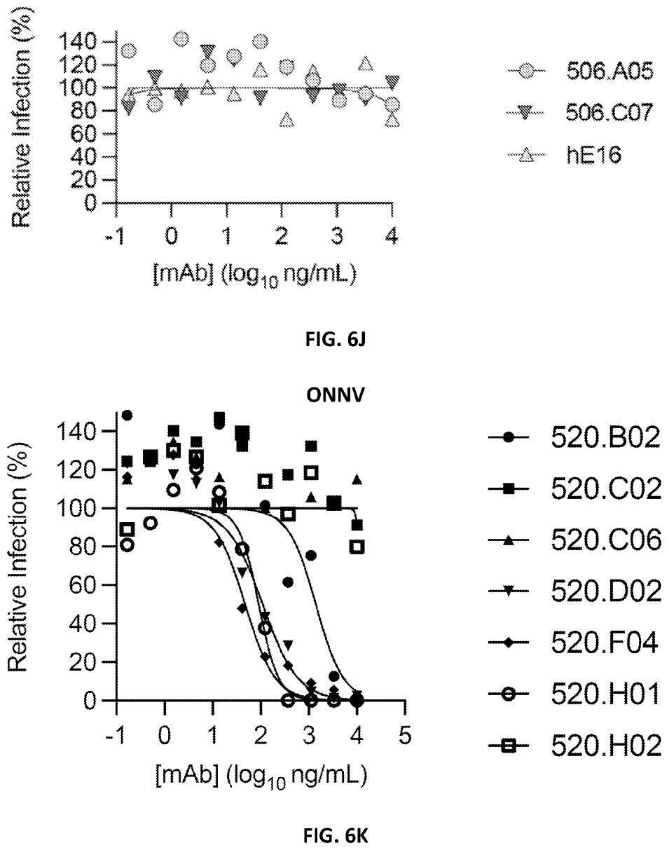

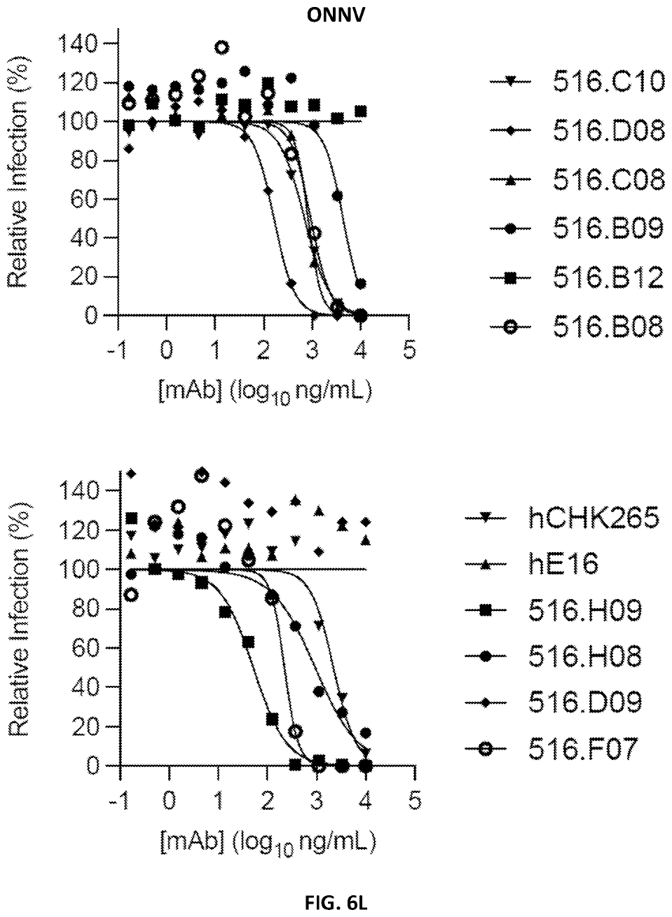

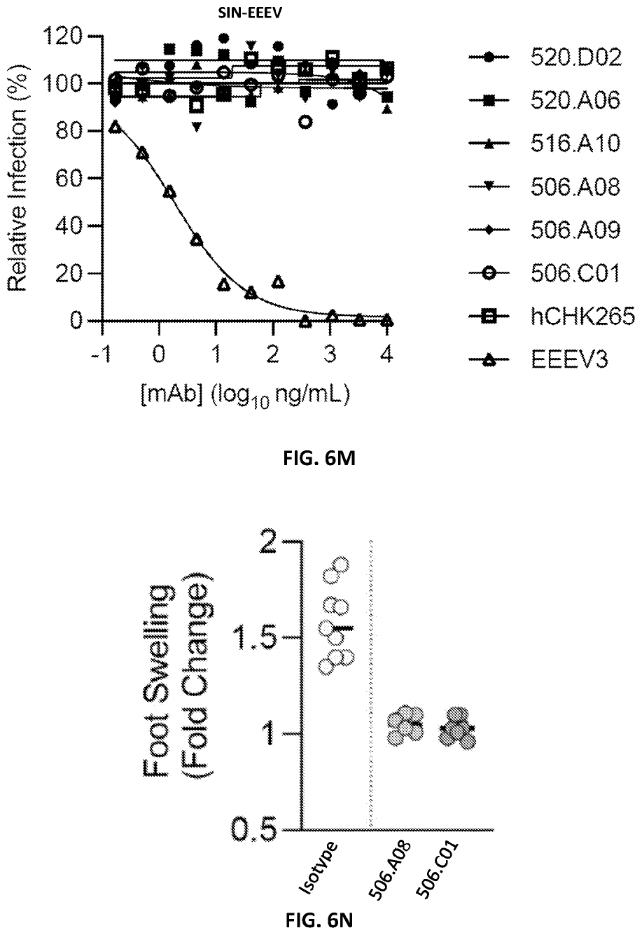

Cross-neutralizing activity were tested for the human mAbs 506.A08, 506.A09, 506.C01, 516.A10, 520.D05, 520.A06, and 516.H07 that cross-reacted with three or more alphaviruses. As shown in FIGS. 5C and 5D, except for 506.A08, all of these mAbs neutralized ONNV infection potently, with EC₅₀ values <50 ng/mL. 506.A08 and 506.C01 also inhibited infection of MAYV and RRV to lesser degrees. The mAb 506.C01 demonstrated the greatest neutralization breadth, as it also blocked infection of UNAV and the distantly related Getah virus (GETV), a pattern similar to that of CHK-265. However, 516.A10 did not neutralize infection of SINV-EEEV despite binding to SINV-EEEV-infected cells as shown in FIG. 6M.

To test whether the cross-reactive mAbs could protect against related alphavirus infections, established models of MAYV and RRV pathogenesis in mice were used (Morrison T E et al., Characterization of Ross River virus tropism and virus-induced inflammation in a mouse model of viral arthritis and myositis, J Virol. 80(2):737-49 (2006); Morrison, T. E. et al., A mouse model of chikungunya virus-induced musculoskeletal inflammatory disease: evidence of arthritis, tenosynovitis, myositis, and persistence, Am. J. Pathol., 178:32 40, 2011; Kim A S et al., Pan-protective anti-alphavirus human antibodies target a conserved E1 protein epitope, Cell 184(17):4414-4429 (2021)). 506.C01 and 506.A08 were evaluated as they demonstrated the greatest breadth of binding to cell-surface antigen and cross-neutralization. When administered as prophylaxis, both 506.A08 and 506.C01 reduced MAYV viral burden at 3 days post infection in the ipsilateral ankle and calf. However, only 506.A08 reduced viral burden in the contralateral ankle and calf with several mice below the limit of detection (FIG. 5E), consistent with its greater neutralization potency than 506.C01 against MAYV. In contrast, when mice were inoculated with RRV, marked protection was observed only with 506.A08, with reduced viral burden in all musculoskeletal tissues; 506.C01 exerted a statistically significant yet minor level of protection, consistent with its poorer neutralizing activity against RRV (FIG. 5F).

### Example 7. Structural Analysis of Broadly Neutralizing and Protective mAbs

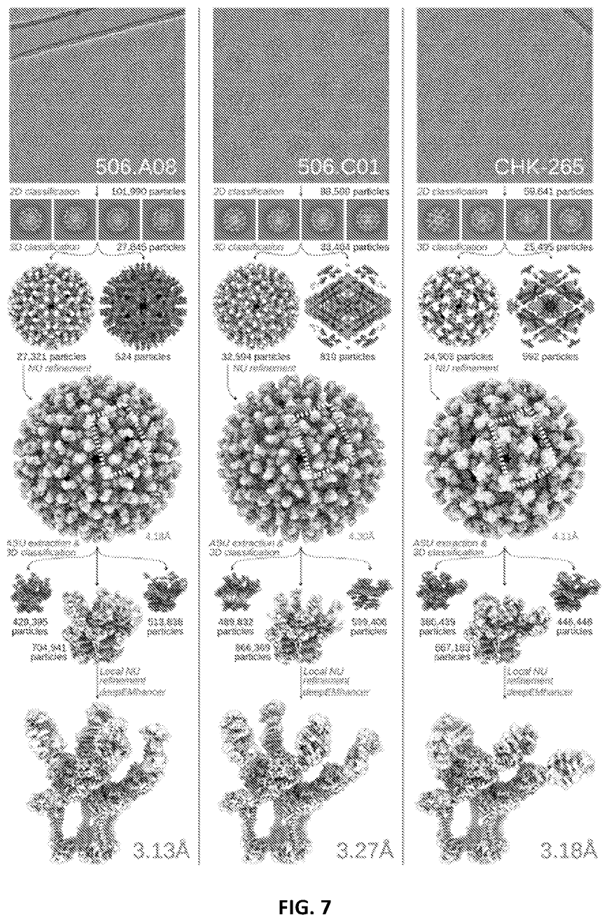

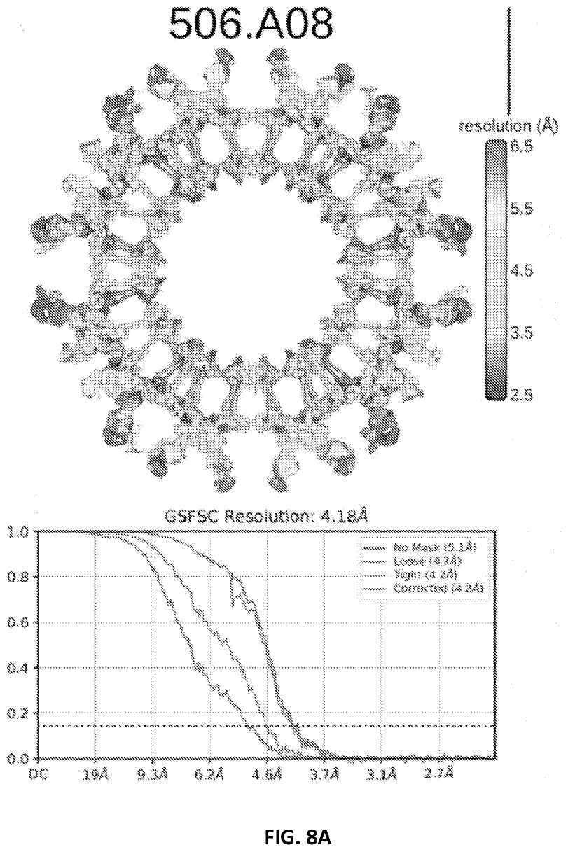

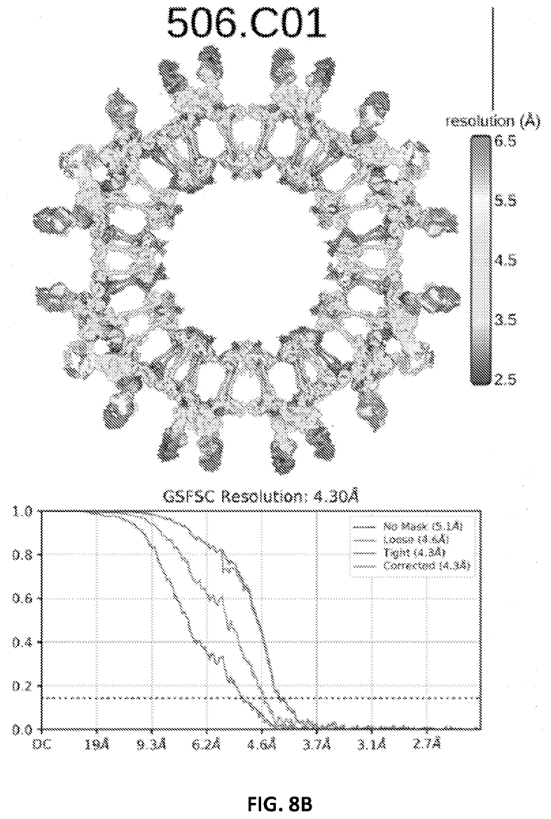

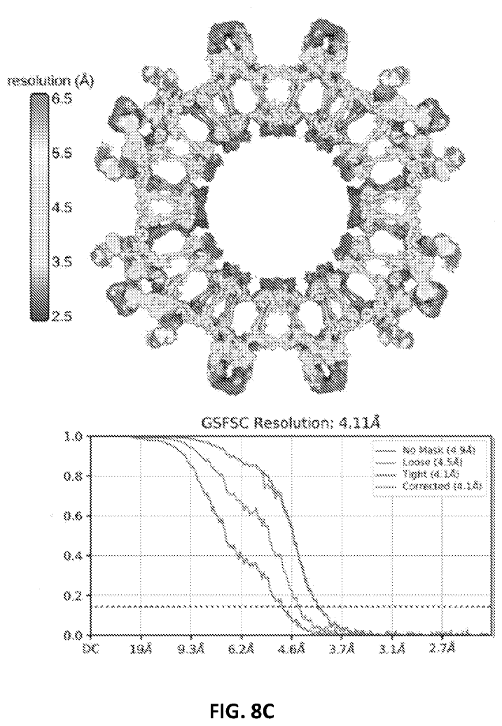

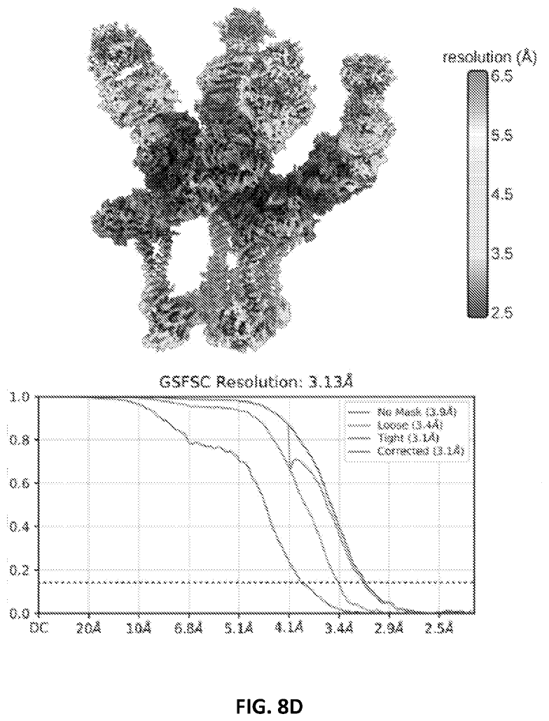

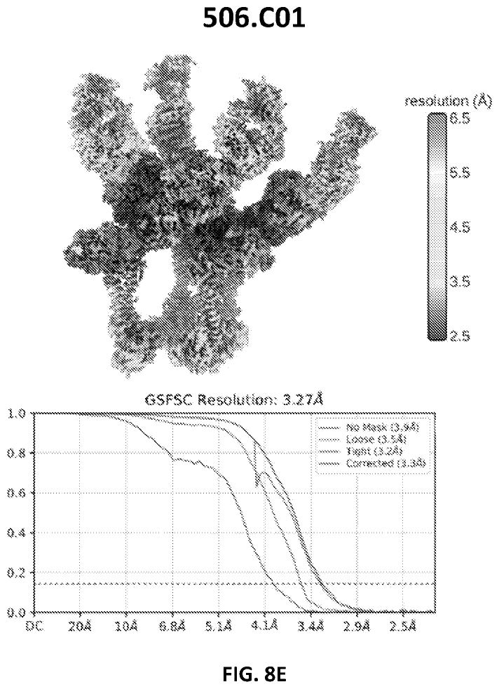

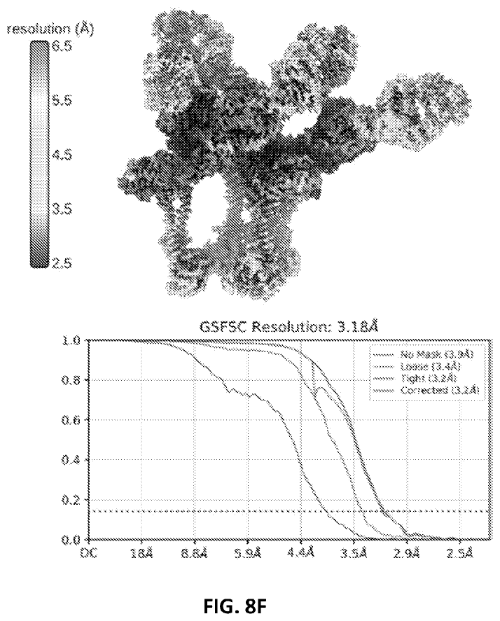

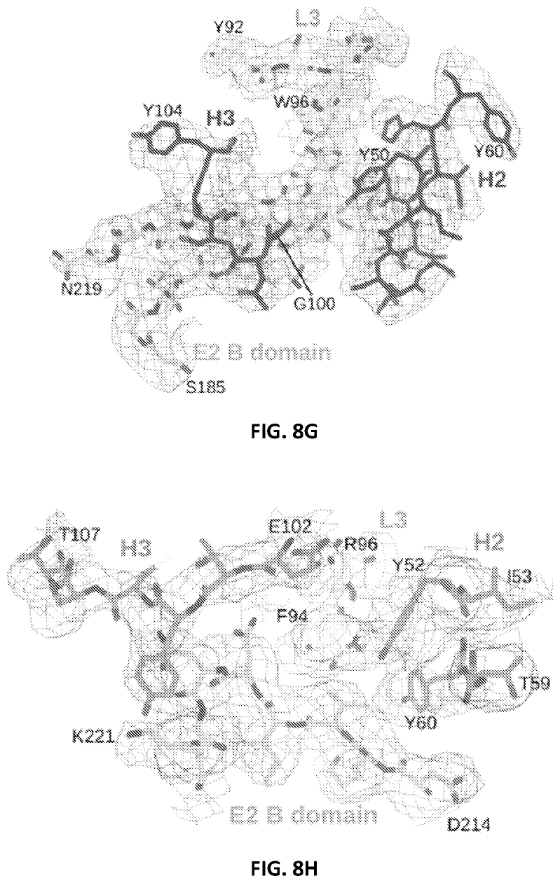

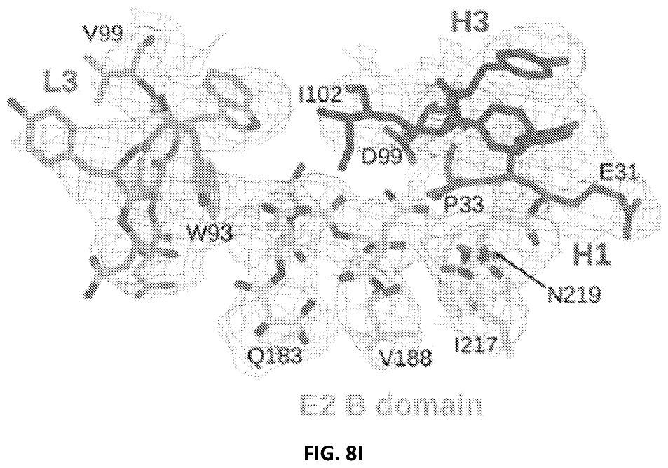

Competition-binding analysis, alanine-scanning mutagenesis, and neutralization escape studies suggested that 506.A08 and 506.C01 target similar epitopes on the B domain of E2. However, given their differing profiles of cross-reactivity shown in FIG. 5A, these mAbs might target overlapping but distinct epitopes, providing an opportunity to map structurally the exact regions on the B domain that are targeted by broadly neutralizing antibodies. In some embodiments, previously generated low-resolution (˜16 Å) cryo-electron microscopy (cryo-EM) reconstruction of the broadly-neutralizing murine mAb CHK-265 bound to CHIKV and intermediate resolution (6.3, 5.3, and 5.3 Å) reconstructions of the broadly-neutralizing human mAb RRV-12 bound to RRV, CHIKV, and MAYV are provided. These structures showed similar mAb binding orientations and footprints in the B domain, with potential crosslinking of A domains in adjacent trimers via framework region contacts. In some embodiments, a higher-resolution cryo-EM reconstructions of CHIKV VLPs complexed with 506.A08 and 506.C01 Fabs, and CHK-265 Fab for reference were generated (FIGS. 6A-6C, 7, and 8A-8C). Following icosahedral reconstruction of Fab-bound VLPs, asymmetric units (n=60 per VLP) were extracted for independent classification and focused refinement to account for deviations from ideal icosahedral geometry (FIG. 7). The resulting reconstructions of CHIKV structural proteins (E1, E2, and capsid) in complex with 506.A08, 506.C01, or CHK-265 Fabs achieved resolutions of 3.13 Å, 3.27 Å, or 3.18 Å, respectively (FIGS. 6D-6F and 8D-I).

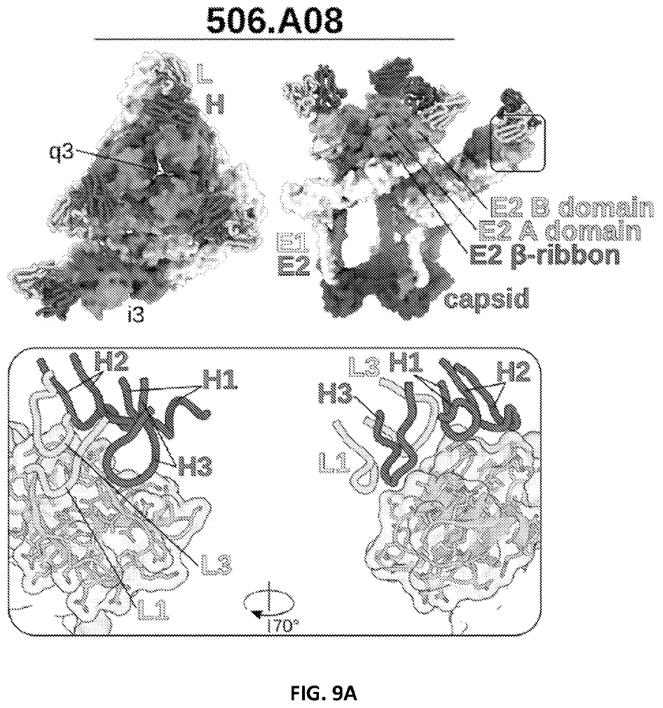

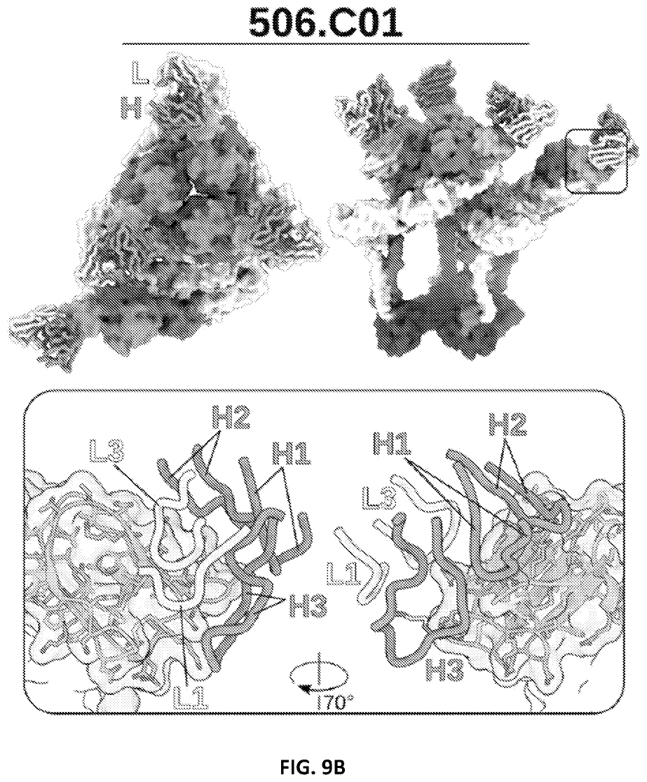

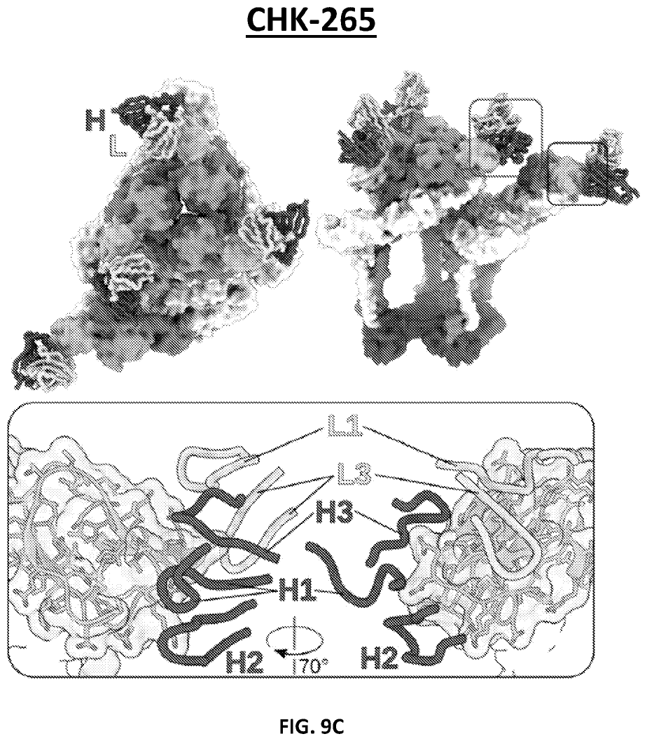

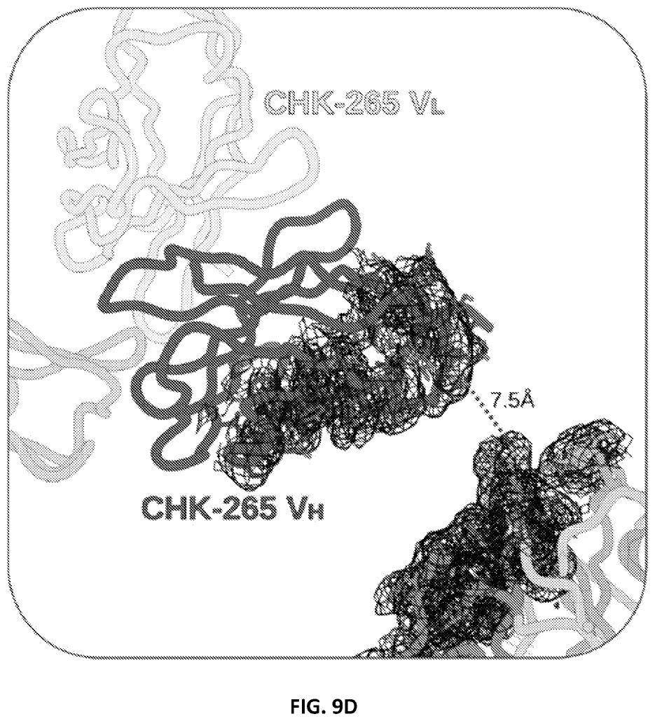

In some embodiments, all three Fabs bound to the B domain of CHIKV E2. However, each one evaluated demonstrated a unique angle of engagement, with 506.A08 positioned upright on the apex of B domain, 506.C01 positioned at a 30° incline on the apex, and CHK-265 binding the lateral tip of B domain nearly parallel to the plane of the viral membrane (FIG. 6D-F). Each Fab likewise established a unique footprint on B domain, with 506.A08 centered on the apex, CHK-265 centered on the lateral tip, and 506.C01 spanning both regions (FIGS. 6G-61 and 9A-9C). 506.C01 achieved this recognition by using an unusually long, disulfide-stabilized CDR-H3, which folded over the lateral tip of B domain and extended toward the viral membrane (FIG. 9B).

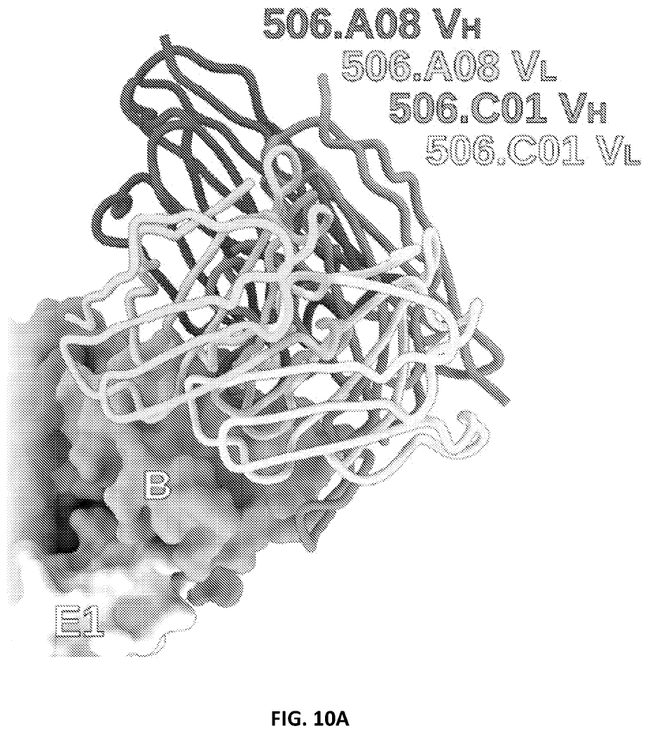

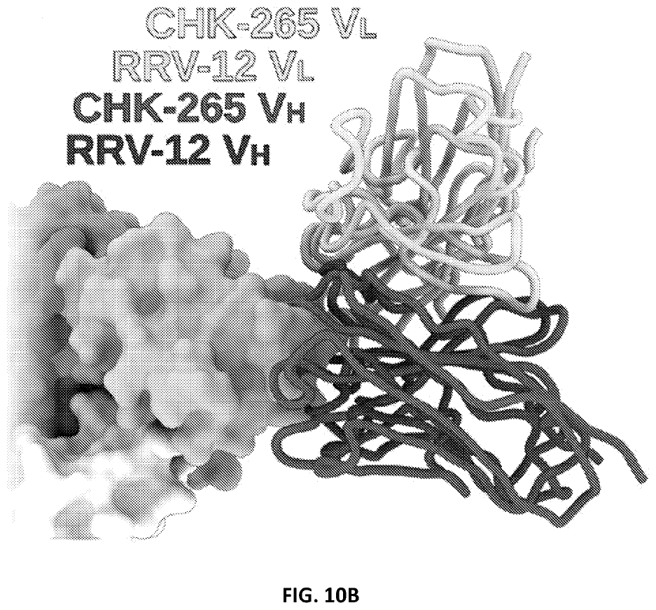

Despite their different approaches to E2, 506.C01 and CHK-265 had similar footprints on the surface of E2 (FIGS. 6H-I). These structural results may explain the cross-reactivity patterns for 506.A08 and 506.C01. Although these mAbs could accommodate a variety of substitutions within their respective epitopes, divergence from CHIKV at particular E2 residues resulted in loss of reactivity. Given their central positions within each epitope and the mutagenesis findings of the disclosure, polymorphisms N187D (BEBV) and T213S/V (GETV/UNAV) likely contribute to the different binding patterns of 506.C01 and 506.A08, respectively (FIG. 6I). Cross-linking of the A domain by any of the three mAbs, including CHK-265, were not observed (FIG. 9D), providing evidence that binding to the B domain alone is sufficient for broadly neutralizing activity. 506.A08 and 506.C01 define unique neutralizing epitopes atop the apex of B domain, whereas other previously characterized broadly-neutralizing mAbs (CHK-265 and RRV-12) approach the lateral tip of the B domain from the side (FIGS. 10A and 10B).

Close contact and buried surface area at Fab/E2 interface are also analyzed based on the cryo-EM model, which are shown by the following Table 5.

TABLE 5
Close contacts & buried surface area at Fab/E2 interfaces

| | | Antibody | Buried surface |
|---|---|---|---|
| | CHIKV E2 | residue(s) (no. | area Å² |
| | residue | close contacts) | (% of interface) |
| 506.A08 | Q184 | | 4.1 (1.0%) |
| | N187 | | 2.5 (0.6%) |
| | K189 | | 13.9 (3.5%) |
| | T191 | | 0.7 (0.2%) |
| | V192 | | 6.7 (1.7%) |
| | N193 | S56^{H}(1) | 32.8 (8.3%) |
| | G194 | T57^{H}(2) | 25.5 (6.5%) |
| | Y199 | | 4.9 (1.2%) |
| | N207 | | 2.8 (0.7%) |
| | G209 | | 4.4 (1.1%) |
| | T212 | | 26.6 (6.7%) |
| | T213 | T94^{L}(1) | 77.9 (19.8%) |
| | D214 | E105^{H}(1) | 41.4 (10.5%) |
| | K215 | E105^{H}(4) | 63.8 (16.2%) |
| | V216 | S102^{H}(2), G103^{H}(2) | 47.8 (12.1%) |
| | N218 | G103^{H}(2) | 38.0 (9.7%) |
| | Total | 15 close contacts | 394 Å² |
| 506.C01 | Q184 | F54^{H}(1), G58^{H}(2) | 83.4 (12.7%) |
| | S185 | D32^{H}(2), F54^{H}(4) | 79.1 (12.1%) |
| | G186 | Y35^{H}(1), K112^{H}(1) | 38.1 (5.8%) |
| | N187 | | 49.8 (7.6%) |
| | K189 | T59^{H}(1) | 34.3 (5.2%) |
| | D214 | Y60^{H}(1) | 42.7 (6.5%) |
| | K215 | | 5.4 (0.8%) |
| | V216 | Y52^{H}(1) | 83.9 (12.8%) |
| | N218 | A91^{L}(1), N92^{L}(4), | 104.4 (15.9%) |
| | | F94^{L}(1), K112^{H}(1) | |
| | N219 | Y104^{H}(4), C105^{H}(4) | 91.3 (13.9%) |
| | C220 | | 1.7 (0.3%) |
| | K221 | | 40.8 (6.2%) |
| | Total | 29 close contacts | 655 Å² |
| CHK-265 | Q183 | | 8.7 (1.5%) |
| | Q184 | C34^{L}(1), I102^{H}(1) | 74.0 (12.8%) |
| | S185 | W93^{L}(6), W98^{L}(2), | 87.9 (15.3%) |
| | | D99^{H}(1), I102^{H}(1) | |
| | G186 | | 45.0 (7.8%) |
| | N187 | D99^{H}(1), F101^{H}(4), | 43.7 (7.6%) |
| | | I102^{H}(2) | |
| | K189 | | 11.3 (2.0%) |
| | C203 | | 1.5 (0.3%) |
| | G204 | | 22.8 (4.0%) |
| | V216 | | 25.5 (4.4%) |
| | N218 | F101^{H}(2) | 59.0 (10.2%) |
| | N219 | P33^{H}(4), Y52^{H}(1) | 117.1 (20.3%) |
| | C220 | | 8.9 (1.6%) |
| | K221 | E57^{H}(3) | 70.7 (12.3%) |
| | Total | 29 close contacts | 576 Å² |

The broadly neutralizing mAbs 506.C01 and 506.A08 bound to the apex of the B domain, partially overlapping with previously identified human cross-reactive mAb, RRV-12. Whereas RRV-12 only partially neutralized RRV and CHIKV infection and had resistant virus fractions at high concentrations in neutralization tests, 506.C01 and 506.A08 completely inhibited infection of CHIKV-37997 or CHIKV-LR 2006. The cross-neutralizing human mAb DC2.M357, which maps to residues K189 and N218 in the B domain, also completely inhibits CHIKV infection, albeit less potently (EC₅₀ of ˜800 ng/ml) than 506.C01 or 506.A08. Certain isolated mAbs of the present disclosure can protect against infection caused by multiple alphaviruses.

Previous structural analyses of CHK-265 and RRV-12 suggested that cross-linking of neighboring trimers via framework region contacts to A domain may be required for broad neutralization. However, neither 506.A08 nor 506.C01 contact neighboring A domains, and little evidence was observed for such cross-linking by CHK-265 on CHIKV VLPs in the higher resolution structure. Although it is possible that CHK-265 contacts E3 of neighboring trimers in mature virions (versus the VLPs, which lack E3), the binding modes of 506.A08 and 506.C01 are incompatible with such contacts. Thus, the analysis suggests that E2 trimer cross-linking is not required for neutralization of related arthritogenic alphaviruses. Furthermore, CHK-265 and RRV-12 both bind the lateral tip of E2 B domain nearly parallel to the viral membrane, whereas 506.A08 and 506.C01 engage the apex of B domain. Within these epitopes, key residue changes were identified in CHIKV E2 that possibly explain the loss of binding for 506.C01 to BEBV (N187D), 506.A08 to GETV (T213S), and 506.A08 to UNAV (T213V).

The examples of the present disclosure describe protective type-specific and broadly neutralizing antibodies against arthritogenic alphaviruses, which are advantageous over related antibodies.

What is claimed is:

1. An antigen binding molecule that binds to an alphavirus, wherein the antigen binding molecule comprises:
a CDR-H1 comprising SEQ ID NO:2, 10, 18, 26, 34, 42, 50, 58, 66, 74, 82, 90, 98, 106, 114, 122, 130, 138, 146, 154, 162, 170, 178, 186, 194, 202, 210, 218, 226, 234, 242, 250, 258, 266, 274, 282, 290 or 298,
a CDR-H2 comprising SEQ ID NO:3, 11, 19, 27, 35, 43, 51, 59, 67, 75, 83, 91, 99, 107, 115, 123, 131, 139, 147, 155, 163, 171, 179, 187, 195, 203, 211, 219, 227, 235, 243, 251, 259, 267, 275, 283, 291 or 299, and
a CDR-H3 comprising SEQ ID NO:4, 12, 20, 28, 36, 44, 52, 60, 68, 76, 84, 92, 100, 108, 116, 124, 132, 140, 148, 156, 164, 172, 180, 188, 196, 204, 212, 220, 228, 236, 244, 252, 260, 268, 276, 284, 292 or 300; and
a CDR-L1 comprising SEQ ID NO:6, 14, 22, 30, 38, 46, 54, 62, 70, 78, 86, 94, 102, 110, 118, 126, 134, 142, 150, 158, 166, 174, 182, 190, 198, 206, 214, 222, 230, 238, 246, 254, 262, 270, 278, 286, 294 or 302,
a CDR-L2 comprising SEQ ID NO:7, 15, 23, 31, 39, 47, 55, 63, 71, 79, 87, 95, 103, 111, 119, 127, 135, 143, 151, 159, 167, 175, 183, 191, 199, 207, 215, 223, 231, 239, 247, 255, 263, 271, 279, 287, 295 or 303, and
a CDR-L3 comprising SEQ ID NO: 8, 16, 24, 32, 40, 48, 56, 64, 72, 80, 88, 96, 104, 112, 120, 128, 136, 144, 152, 160, 168, 176, 184, 192, 200, 208, 216, 224, 232, 240, 248, 256, 264, 272, 280, 288, 296 or 304.

2. The antigen binding molecule of claim 1, wherein:
the antigen binding molecule comprises a heavy chain variable region, which comprises an amino acid sequence that is at least 80% identical to a sequence selected from SEQ ID NOs:1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289 and 297; and
the antigen binding molecule comprises a light chain variable region, which comprises an amino acid sequence that is at least 80% identical to a sequence selected from SEQ ID NOs:5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293 and 301.

3. The antigen binding molecule of claim 2, wherein:
the heavy chain variable region comprises an amino acid sequence selected from SEQ ID NOs:1, 9, 17, 25, 33, 41, 49, 57, 65, 73, 81, 89, 97, 105, 113, 121, 129, 137, 145, 153, 161, 169, 177, 185, 193, 201, 209, 217, 225, 233, 241, 249, 257, 265, 273, 281, 289 and 297; and
the light chain variable region comprises an amino acid sequence selected from SEQ ID NOs:5, 13, 21, 29, 37, 45, 53, 61, 69, 77, 85, 93, 101, 109, 117, 125, 133, 141, 149, 157, 165, 173, 181, 189, 197, 205, 213, 221, 229, 237, 245, 253, 261, 269, 277, 285, 293 and 301.

4. The antigen binding molecule of any one of claims 1 to 3, wherein:
each of CDR-H1, CDR-H2, CDR-H3, CDR-L1, CDR-L2, CDR-L3 is from a same row of Table 1, wherein Table 1 comprises rows 1-38:
TABLE 1

| | CDR-H1 | CDR-H2 | CDR-H3 | CDR-L1 | CDR-L2 | CDR-L3 |
|---|---|---|---|---|---|---|
| 1 | SEQ ID NO: 2 | SEQ ID NO: 3 | SEQ ID NO: 4 | SEQ ID NO: 6 | SEQ ID NO: 7 | SEQ ID NO: 8 |
| 2 | SEQ ID NO: 10 | SEQ ID NO: 11 | SEQ ID NO: 12 | SEQ ID NO: 14 | SEQ ID NO: 15 | SEQ ID NO: 16 |
| 3 | SEQ ID NO: 18 | SEQ ID NO: 19 | SEQ ID NO: 20 | SEQ ID NO: 22 | SEQ ID NO: 23 | SEQ ID NO: 24 |
| 4 | SEQ ID NO: 26 | SEQ ID NO: 27 | SEQ ID NO: 28 | SEQ ID NO: 30 | SEQ ID NO: 31 | SEQ ID NO: 32 |
| 5 | SEQ ID NO: 34 | SEQ ID NO: 35 | SEQ ID NO: 36 | SEQ ID NO: 38 | SEQ ID NO: 39 | SEQ ID NO: 40 |
| 6 | SEQ ID NO: 42 | SEQ ID NO: 43 | SEQ ID NO: 44 | SEQ ID NO: 46 | SEQ ID NO: 47 | SEQ ID NO: 48 |
| 7 | SEQ ID NO: 50 | SEQ ID NO: 51 | SEQ ID NO: 52 | SEQ ID NO: 54 | SEQ ID NO: 55 | SEQ ID NO: 56 |
| 8 | SEQ ID NO: 58 | SEQ ID NO: 59 | SEQ ID NO: 60 | SEQ ID NO: 62 | SEQ ID NO: 63 | SEQ ID NO: 64 |
| 9 | SEQ ID NO: 66 | SEQ ID NO: 67 | SEQ ID NO: 68 | SEQ ID NO: 70 | SEQ ID NO: 71 | SEQ ID NO: 72 |
| 10 | SEQ ID NO: 74 | SEQ ID NO: 75 | SEQ ID NO: 76 | SEQ ID NO: 78 | SEQ ID NO: 79 | SEQ ID NO: 80 |
| 11 | SEQ ID NO: 82 | SEQ ID NO: 83 | SEQ ID NO: 84 | SEQ ID NO: 86 | SEQ ID NO: 87 | SEQ ID NO: 88 |
| 12 | SEQ ID NO: 90 | SEQ ID NO: 91 | SEQ ID NO: 92 | SEQ ID NO: 94 | SEQ ID NO: 95 | SEQ ID NO: 96 |
| 13 | SEQ ID NO: 98 | SEQ ID NO: 99 | SEQ ID NO: 100 | SEQ ID NO: 102 | SEQ ID NO: 103 | SEQ ID NO: 104 |
| 14 | SEQ ID NO: 106 | SEQ ID NO: 107 | SEQ ID NO: 108 | SEQ ID NO: 110 | SEQ ID NO: 111 | SEQ ID NO: 112 |
| 15 | SEQ ID NO: 114 | SEQ ID NO: 115 | SEQ ID NO: 116 | SEQ ID NO: 118 | SEQ ID NO: 119 | SEQ ID NO: 120 |
| 16 | SEQ ID NO: 122 | SEQ ID NO: 123 | SEQ ID NO: 124 | SEQ ID NO: 126 | SEQ ID NO: 127 | SEQ ID NO: 128 |
| 17 | SEQ ID NO: 130 | SEQ ID NO: 131 | SEQ ID NO: 132 | SEQ ID NO: 134 | SEQ ID NO: 135 | SEQ ID NO: 136 |
| 18 | SEQ ID NO: 138 | SEQ ID NO: 139 | SEQ ID NO: 140 | SEQ ID NO: 142 | SEQ ID NO: 143 | SEQ ID NO: 144 |
| 19 | SEQ ID NO: 146 | SEQ ID NO: 147 | SEQ ID NO: 148 | SEQ ID NO: 150 | SEQ ID NO: 151 | SEQ ID NO: 152 |
| 20 | SEQ ID NO: 154 | SEQ ID NO: 155 | SEQ ID NO: 156 | SEQ ID NO: 158 | SEQ ID NO: 159 | SEQ ID NO: 160 |
| 21 | SEQ ID NO: 162 | SEQ ID NO: 163 | SEQ ID NO: 164 | SEQ ID NO: 166 | SEQ ID NO: 167 | SEQ ID NO: 168 |
| 22 | SEQ ID NO: 170 | SEQ ID NO: 171 | SEQ ID NO: 172 | SEQ ID NO: 174 | SEQ ID NO: 175 | SEQ ID NO: 176 |
| 23 | SEQ ID NO: 178 | SEQ ID NO: 179 | SEQ ID NO: 180 | SEQ ID NO: 182 | SEQ ID NO: 183 | SEQ ID NO: 184 |
| 24 | SEQ ID NO: 186 | SEQ ID NO: 187 | SEQ ID NO: 188 | SEQ ID NO: 190 | SEQ ID NO: 191 | SEQ ID NO: 192 |
| 25 | SEQ ID NO: 194 | SEQ ID NO: 195 | SEQ ID NO: 196 | SEQ ID NO: 198 | SEQ ID NO: 199 | SEQ ID NO: 200 |
| 26 | SEQ ID NO: 202 | SEQ ID NO: 203 | SEQ ID NO: 204 | SEQ ID NO: 206 | SEQ ID NO: 207 | SEQ ID NO: 208 |
| 27 | SEQ ID NO: 210 | SEQ ID NO: 211 | SEQ ID NO: 212 | SEQ ID NO: 214 | SEQ ID NO: 215 | SEQ ID NO: 216 |
| 28 | SEQ ID NO: 218 | SEQ ID NO: 219 | SEQ ID NO: 220 | SEQ ID NO: 222 | SEQ ID NO: 223 | SEQ ID NO: 224 |
| 29 | SEQ ID NO: 226 | SEQ ID NO: 227 | SEQ ID NO: 228 | SEQ ID NO: 230 | SEQ ID NO: 231 | SEQ ID NO: 232 |
| 30 | SEQ ID NO: 234 | SEQ ID NO: 235 | SEQ ID NO: 236 | SEQ ID NO: 238 | SEQ ID NO: 239 | SEQ ID NO: 240 |
| 31 | SEQ ID NO: 242 | SEQ ID NO: 243 | SEQ ID NO: 244 | SEQ ID NO: 246 | SEQ ID NO: 247 | SEQ ID NO: 248 |
| 32 | SEQ ID NO: 250 | SEQ ID NO: 251 | SEQ ID NO: 252 | SEQ ID NO: 254 | SEQ ID NO: 255 | SEQ ID NO: 256 |
| 33 | SEQ ID NO: 258 | SEQ ID NO: 259 | SEQ ID NO: 260 | SEQ ID NO: 262 | SEQ ID NO: 263 | SEQ ID NO: 264 |
| 34 | SEQ ID NO: 266 | SEQ ID NO: 267 | SEQ ID NO: 268 | SEQ ID NO: 270 | SEQ ID NO: 271 | SEQ ID NO: 272 |
| 35 | SEQ ID NO: 274 | SEQ ID NO: 275 | SEQ ID NO: 276 | SEQ ID NO: 278 | SEQ ID NO: 279 | SEQ ID NO: 280 |
| 36 | SEQ ID NO: 282 | SEQ ID NO: 283 | SEQ ID NO: 284 | SEQ ID NO: 286 | SEQ ID NO: 287 | SEQ ID NO: 288 |
| 37 | SEQ ID NO: 290 | SEQ ID NO: 291 | SEQ ID NO: 292 | SEQ ID NO: 294 | SEQ ID NO: 295 | SEQ ID NO: 296 |
| 38 | SEQ ID NO: 298 | SEQ ID NO: 299 | SEQ ID NO: 300 | SEQ ID NO: 302 | SEQ ID NO: 303 | SEQ ID NO: 304. |

5. The antigen binding molecule of any one of claims 1 to 4, wherein:
the heavy chain variable region comprises an amino acid sequence at least 80% identical to SEQ ID NO:1, and the light chain variable region comprises an amino acid sequence at least 80% identical to SEQ ID NO:5;
the heavy chain variable region comprises an amino acid sequence at least 80% identical to SEQ ID NO:9, and the light chain variable region comprises an amino acid sequence at least 80% identical to SEQ ID NO:13;
the heavy chain variable region comprises an amino acid sequence at least 80% identical to SEQ ID NO:17, and the light chain variable region comprises an amino acid sequence at least 80% identical to SEQ ID NO:21;
the heavy chain variable region comprises an amino acid sequence at least 80% identical to SEQ ID NO:25, and the light chain variable region comprises an amino acid sequence at least 80% identical to SEQ ID NO:29;
the heavy chain variable region comprises an amino acid sequence at least 80% identical to SEQ ID NO:33, and the light chain variable region comprises an amino acid sequence at least 80% identical to SEQ ID NO:37;
the heavy chain variable region comprises an amino acid sequence at least 80% identical to SEQ ID NO:41, and the light chain variable region comprises an amino acid sequence at least 80% identical to SEQ ID NO:45;
the heavy chain variable region comprises an amino acid sequence at least 80% identical to SEQ ID NO:49, and the light chain variable region comprises an amino acid sequence at least 80% identical to SEQ ID NO:53;
the heavy chain variable region comprises an amino acid sequence at least 80% identical to SEQ ID NO:57, and the light chain variable region comprises an amino acid sequence at least 80% identical to SEQ ID NO:61;
the heavy chain variable region comprises an amino acid sequence at least 80% identical to SEQ ID NO:65, and the light chain variable region comprises an amino acid sequence at least 80% identical to SEQ ID NO:69;
the heavy chain variable region comprises an amino acid sequence at least 80% identical to SEQ ID NO:73, and the light chain variable region comprises an amino acid sequence at least 80% identical to SEQ ID NO:77;
the heavy chain variable region comprises an amino acid sequence at least 80% identical to SEQ ID NO:81, and the light chain variable region comprises an amino acid sequence at least 80% identical to SEQ ID NO:85;
the heavy chain variable region comprises an amino acid sequence at least 80% identical to SEQ ID NO:89, and the light chain variable region comprises an amino acid sequence at least 80% identical to SEQ ID NO:93;
the heavy chain variable region comprises an amino acid sequence at least 80% identical to SEQ ID NO:97, and the light chain variable region comprises an amino acid sequence at least 80% identical to SEQ ID NO:101;
the heavy chain variable region comprises an amino acid sequence at least 80% identical to SEQ ID NO:105, and the light chain variable region comprises an amino acid sequence at least 80% identical to SEQ ID NO:109;
the heavy chain variable region comprises an amino acid sequence at least 80% identical to SEQ ID NO:113, and the light chain variable region comprises an amino acid sequence at least 80% identical to SEQ ID NO:117;
the heavy chain variable region comprises an amino acid sequence at least 80% identical to SEQ ID NO:121, and the light chain variable region comprises an amino acid sequence at least 80% identical to SEQ ID NO:125;
the heavy chain variable region comprises an amino acid sequence at least 80% identical to SEQ ID NO:129, and the light chain variable region comprises an amino acid sequence at least 80% identical to SEQ ID NO:133;
the heavy chain variable region comprises an amino acid sequence at least 80% identical to SEQ ID NO:127, and the light chain variable region comprises an amino acid sequence at least 80% identical to SEQ ID NO:141;
the heavy chain variable region comprises an amino acid sequence at least 80% identical to SEQ ID NO:145, and the light chain variable region comprises an amino acid sequence at least 80% identical to SEQ ID NO:149;
the heavy chain variable region comprises an amino acid sequence at least 80% identical to SEQ ID NO:153, and the light chain variable region comprises an amino acid sequence at least 80% identical to SEQ ID NO:157;
the heavy chain variable region comprises an amino acid sequence at least 80% identical to SEQ ID NO:161, and the light chain variable region comprises an amino acid sequence at least 80% identical to SEQ ID NO:165;
the heavy chain variable region comprises an amino acid sequence at least 80% identical to SEQ ID NO:169, and the light chain variable region comprises an amino acid sequence at least 80% identical to SEQ ID NO:173;
the heavy chain variable region comprises an amino acid sequence at least 80% identical to SEQ ID NO:177, and the light chain variable region comprises an amino acid sequence at least 80% identical to SEQ ID NO:181;
the heavy chain variable region comprises an amino acid sequence at least 80% identical to SEQ ID NO:185, and the light chain variable region comprises an amino acid sequence at least 80% identical to SEQ ID NO:189;
the heavy chain variable region comprises an amino acid sequence at least 80% identical to SEQ ID NO:193, and the light chain variable region comprises an amino acid sequence at least 80% identical to SEQ ID NO:197;
the heavy chain variable region comprises an amino acid sequence at least 80% identical to SEQ ID NO:201, and the light chain variable region comprises an amino acid sequence at least 80% identical to SEQ ID NO:205;
the heavy chain variable region comprises an amino acid sequence at least 80% identical to SEQ ID NO:209, and the light chain variable region comprises an amino acid sequence at least 80% identical to SEQ ID NO:213;
the heavy chain variable region comprises an amino acid sequence at least 80% identical to SEQ ID NO:217, and the light chain variable region comprises an amino acid sequence at least 80% identical to SEQ ID NO:221;
the heavy chain variable region comprises an amino acid sequence at least 80% identical to SEQ ID NO:225, and the light chain variable region comprises an amino acid sequence at least 80% identical to SEQ ID NO:229;
the heavy chain variable region comprises an amino acid sequence at least 80% identical to SEQ ID NO:233, and the light chain variable region comprises an amino acid sequence at least 80% identical to SEQ ID NO:237;
the heavy chain variable region comprises an amino acid sequence at least 80% identical to SEQ ID NO:241, and the light chain variable region comprises an amino acid sequence at least 80% identical to SEQ ID NO:245;
the heavy chain variable region comprises an amino acid sequence at least 80% identical to SEQ ID NO:249, and the light chain variable region comprises an amino acid sequence at least 80% identical to SEQ ID NO:253;
the heavy chain variable region comprises an amino acid sequence at least 80% identical to SEQ ID NO:257, and the light chain variable region comprises an amino acid sequence at least 80% identical to SEQ ID NO:261;
the heavy chain variable region comprises an amino acid sequence at least 80% identical to SEQ ID NO:265, and the light chain variable region comprises an amino acid sequence at least 80% identical to SEQ ID NO:269;
the heavy chain variable region comprises an amino acid sequence at least 80% identical to SEQ ID NO:273, and the light chain variable region comprises an amino acid sequence at least 80% identical to SEQ ID NO:277;
the heavy chain variable region comprises an amino acid sequence at least 80% identical to SEQ ID NO:281, and the light chain variable region comprises an amino acid sequence at least 80% identical to SEQ ID NO:285;
the heavy chain variable region comprises an amino acid sequence at least 80% identical to SEQ ID NO:289, and the light chain variable region comprises an amino acid sequence at least 80% identical to SEQ ID NO:293; or
the heavy chain variable region comprises an amino acid sequence at least 80% identical to SEQ ID NO:297, and the light chain variable region comprises an amino acid sequence at least 80% identical to SEQ ID NO:301.

6. The antigen binding molecule of any one of claims 1 to 5, wherein the antigen binding molecule is an antibody or an antibody derivative.

7. The antigen binding molecule of claim 6, wherein the antigen binding molecule is an antibody.

8. The antigen binding molecule of claim 6, wherein the antigen binding molecule is an antibody derivative.

9. The antigen binding molecule of claim 8, wherein the antibody derivative is an antibody fragment or a chimeric antibody.

10. The antigen binding molecule of claim 9, wherein the antibody fragment is Fab, Fab′, F(ab′)₂, Fv, dsFv, or single chain variable fragment (scFv).

11. The antigen binding molecule of any one of claims 1 to 10, wherein the antigen binding molecule binds to Chikungunya virus (CHIKV) and at least one additional alphavirus selected from O'nyong'nyong virus (ONNV), Mayaro virus (MAYV), Ross River virus (RRV), Una virus (UNAV), Bebaru virus (BEBV), Getah virus (GETV), Eastern equine encephalitis (EEEV), Venezuelan equine encephalitis (VEEV) and Western equine encephalitis (WEEV).

12. The antigen binding molecule of claim 11, wherein the antigen binding molecule binds to CHIKV and ONNV.

13. The antigen binding molecule of claim 11, wherein the antigen binding molecule binds to CHIKV and MAYV.

14. The antigen binding molecule of claim 11, wherein the antigen binding molecule binds to CHIKV and at least one of UNAV, RRV and BEBV.

15. The antigen binding molecule of claim 11, wherein the antigen binding molecule binds to CHIKV, ONNV, MAYV and RRV.

16. The antigen binding molecule of claim 11, wherein the antigen binding molecule binds to CHIKV, ONNV, BEBV and EEEV.

17. The antigen binding molecule of any one of claims 1 to 16, wherein the antigen binding molecule has inhibitory activity of EC₅₀<100 ng/mL measured by focus reduction neutralization tests (FRNTs) against CHIKV-37997 and CHIKV-LR 2006.

18. The antigen binding molecule of claim 17, wherein the antigen binding molecule has inhibitory activity of EC₅₀<10 ng/ml measured by FRNTs against CHIKV-37997 and CHIKV-LR 2006.

19. A pharmaceutical composition comprising the antigen binding molecule of any one of claims 1 to 18.

20. A method of treating a viral infection in a subject in need thereof, comprising administering to the subject a therapeutically effective amount of the antigen binding molecule of any one of claims 1 to 18 or the pharmaceutical composition of claim 19.

21. The method of treating of claim 20, wherein the viral infection is caused by an alphavirus.

22. The method of claim 21, wherein the alphavirus is Chikungunya virus (CHIKV), O'nyong'nyong virus (ONNV), Mayaro virus (MAYV), Ross River virus (RRV), Una virus (UNAV), Bebaru virus (BEBV), Getah virus (GETV), Eastern equine encephalitis (EEEV), Venezuelan equine encephalitis (VEEV), Western equine encephalitis (WEEV), or a combination thereof.


SEQUENCE LISTING (306 sequences)

SEQ ID NO: 1 - protein, 122 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGEAWFGELSSFDYWGQGTLVTVSS

SEQ ID NO: 2 - protein, 8 aa, Homo sapiens
GFTFSSYG

SEQ ID NO: 3 - protein, 8 aa, Homo sapiens
ISYDGSNK

SEQ ID NO: 4 - protein, 15 aa, Homo sapiens
AKGEAWFGELSSFDY

SEQ ID NO: 5 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSSFTFGPGTKVDIK

SEQ ID NO: 6 - protein, 7 aa, Homo sapiens
QSVSSSY

SEQ ID NO: 7 - skipped

SEQ ID NO: 8 - protein, 9 aa, Homo sapiens
QQYGSSSFT

SEQ ID NO: 9 - protein, 127 aa, Homo sapiens
QVQLVESGGGVVQPGRSLRLSCAASGFTFSNYGIHWVRQAPGKGLEWVAVISYDGTNKYYADSVKGRFAISRDNSKNTLYLRMNSLRAEDTAVYYCAKVVEWYCSGGSCYYSGMDVWGQGTTVTVSS

SEQ ID NO: 10 - protein, 8 aa, Homo sapiens
GFTFSNYG

SEQ ID NO: 11 - protein, 8 aa, Homo sapiens
ISYDGTNK

SEQ ID NO: 12 - protein, 20 aa, Homo sapiens
AKVVEWYCSGGSCYYSGMDV

SEQ ID NO: 13 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 14 - protein, 11 aa, Homo sapiens
QSLLHSNGYNY

SEQ ID NO: 15 - skipped

SEQ ID NO: 16 - protein, 9 aa, Homo sapiens
MQALQTPYT

SEQ ID NO: 17 - protein, 122 aa, Homo sapiens
QVQLVQSGSELQKPGASVKVSCKASGYTFTENAIIWVRQAPGQGLEWMGRIIRYPGRPTIAQGFTGRFGFSLDTSINTAYLEISNLKAEDTAFFYCAREYCDSSSCSSFDYWGQGTLVTVSS

SEQ ID NO: 18 - protein, 8 aa, Homo sapiens
GYTFTENA

SEQ ID NO: 19 - protein, 8 aa, Homo sapiens
IIRYPGRP

SEQ ID NO: 20 - protein, 15 aa, Homo sapiens
AREYCDSSSCSSFDY

SEQ ID NO: 21 - protein, 110 aa, Homo sapiens
QSVLTQPPSASGSPGQSVTISCTGTNNDVGIYSYVSWYQQHPGKAPKLMIYEVSKRPSGVPDRFSGSKSGNTASLTVSGLQADDEADYYCSSYAGSNSYVFGSGTKVTVL

SEQ ID NO: 22 - protein, 9 aa, Homo sapiens
NNDVGIYSY

SEQ ID NO: 23 - skipped

SEQ ID NO: 24 - protein, 10 aa, Homo sapiens
SSYAGSNSYV

SEQ ID NO: 25 - protein, 123 aa, Homo sapiens
EVQLVESGGGVVRPGGSLRLSCAASGFTFDDYGMSWVRQAPGKGLEWVSGLNWNGGSTGYADSVKGRFTISRDNAKNSLYLQMNSLRAEDTALYHCARMTVVAAGTSWYLDLWGRGTLVTVSS

SEQ ID NO: 26 - protein, 8 aa, Homo sapiens
GFTFDDYG

SEQ ID NO: 27 - protein, 8 aa, Homo sapiens
LNWNGGST

SEQ ID NO: 28 - protein, 16 aa, Homo sapiens
ARMTVVAAGTSWYLDL

SEQ ID NO: 29 - protein, 109 aa, Homo sapiens
DIVLTQSPGTLSLSPGERATLSCRASQSVSSSYLAWYQQKPGQAPRLLIYDASSRATGIPDRFSGSGSGTDFTLTISRLEPEDFAVYYCQQYGSSPLDTFGQGTKLEIK

SEQ ID NO: 30 - protein, 7 aa, Homo sapiens
QSVSSSY

SEQ ID NO: 31 - skipped

SEQ ID NO: 32 - protein, 10 aa, Homo sapiens
QQYGSSPLDT

SEQ ID NO: 33 - protein, 123 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLGWELLPGSWFDPWGQGTLVTVSS

SEQ ID NO: 34 - protein, 8 aa, Homo sapiens
GFTFSSYA

SEQ ID NO: 35 - protein, 8 aa, Homo sapiens
ISGSGGST

SEQ ID NO: 36 - protein, 16 aa, Homo sapiens
AKDLGWELLPGSWFDP

SEQ ID NO: 37 - protein, 107 aa, Homo sapiens
DIQMTQSPSSLSASVGDRVTITCRASQSISSYLNWYQQKPGKAPKLLIYAASSLQSGVPSRFSGSRSGTDFTLTISSLQPEDFATYYCQQSYSTPYTFGQGTKLEIK

SEQ ID NO: 38 - protein, 6 aa, Homo sapiens
QSISSY

SEQ ID NO: 39 - skipped

SEQ ID NO: 40 - protein, 9 aa, Homo sapiens
QQSYSTPYT

SEQ ID NO: 41 - protein, 121 aa, Homo sapiens
QVQLVESGGGVVQPGRSLRLSCAASGFTFSSYGMHWVRQAPGKGLEWVAVISYDGSNDYYADSVKGRFTISRDNSKNTLYLQMNSLRAEDTAVYYCAKCVWFGELSPFDYWGQGTLVTVSS

SEQ ID NO: 42 - protein, 8 aa, Homo sapiens
GFTFSSYG

SEQ ID NO: 43 - protein, 8 aa, Homo sapiens
ISYDGSND

SEQ ID NO: 44 - protein, 14 aa, Homo sapiens
AKCVWFGELSPFDY

SEQ ID NO: 45 - protein, 108 aa, Homo sapiens
DIQMTQSPSSLSASVGDRVTITCRASQGISNYFAWYQQKPGKVPKLLIYAASTLQSGVPSRFSGSGSGTDFTLTISSLQPEDVATYYCQKYNSAPGITFGPGTKVDIK

SEQ ID NO: 46 - protein, 6 aa, Homo sapiens
QGISNY

SEQ ID NO: 47 - skipped

SEQ ID NO: 48 - protein, 10 aa, Homo sapiens
QKYNSAPGIT

SEQ ID NO: 49 - protein, 119 aa, Homo sapiens
QVQLQESGPGLVKPSQTLSLTCTVSGGSISSGGYYWSWIRQHPGKGLEWIGYIFYSGSTYYNPSLKSRVTISVDTSKNQFSLKLSSVTAADTAVYYCARDRGGYCFDYWGQGTLVTVSS

SEQ ID NO: 50 - protein, 10 aa, Homo sapiens
GGSISSGGYY

SEQ ID NO: 51 - protein, 7 aa, Homo sapiens
IFYSGST

SEQ ID NO: 52 - protein, 11 aa, Homo sapiens
ARDRGGYCFDY

SEQ ID NO: 53 - protein, 107 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLPPTFGQGTKLEIK

SEQ ID NO: 54 - protein, 6 aa, Homo sapiens
QDISNY

SEQ ID NO: 55 - skipped

SEQ ID NO: 56 - protein, 9 aa, Homo sapiens
QQYDNLPPT

SEQ ID NO: 57 - protein, 114 aa, Homo sapiens
QVQLQQWGAGLLKPSETLSLTCAVYGGSFSGYILSWIRQPPGKGLEWIGEINHRGSTSYNPSLKSRVTISLDTSKNHFSLKLSSVTAADTAVYYCAKSPYMDVWGKGTTVTVSS

SEQ ID NO: 58 - protein, 8 aa, Homo sapiens
GGSFSGYI

SEQ ID NO: 59 - protein, 7 aa, Homo sp.
INHRGST

SEQ ID NO: 60 - protein, 8 aa, Homo sapiens
AKSPYMDV

SEQ ID NO: 61 - protein, 108 aa, Homo sapiens
EIVLTQSPGTLSLSPGERATLSCRASQSVSSSYFAWYQQKPGQAPRLLIYGASSRATGIPDRFSGGGSGTDFTLTISRLEPEDFAVYYCQQYVSSPYTFGQGTKLEIK

SEQ ID NO: 62 - protein, 7 aa, Homo sapiens
QSVSSSY

SEQ ID NO: 63 - skipped

SEQ ID NO: 64 - protein, 9 aa, Homo sapiens
QQYVSSPYT

SEQ ID NO: 65 - protein, 119 aa, Homo sapiens
QVQLVESGGGVVQPGRSLRLSCAASGFTFSSYAMHWVRQAPGKGLEWVALVSYDGSNKYYADSVKGRFTISRDNSKNTLYLQMNSLRAEDTAVYYCARAEWELGSFDPWGQGTLVTVSS

SEQ ID NO: 66 - protein, 8 aa, Homo sapiens
GFTFSSYA

SEQ ID NO: 67 - protein, 8 aa, Homo sapiens
VSYDGSNK

SEQ ID NO: 68 - protein, 12 aa, Homo sapiens
ARAEWELGSFDP

SEQ ID NO: 69 - protein, 108 aa, Homo sapiens
EIVLTQSPGTLSLSPGERATLSCRASQSVSSSYLAWYQQKPGQAPRLLIYGASSRATGIPDRLSGSGSGTDFTLTISRLEPEDFAVYYCQQYGSSPLTFGQGTKVEIK

SEQ ID NO: 70 - protein, 7 aa, Homo sapiens
QSVSSSY

SEQ ID NO: 71 - skipped

SEQ ID NO: 72 - protein, 9 aa, Homo sapiens
QQYGSSPLT

SEQ ID NO: 73 - protein, 119 aa, Homo sapiens
QVQLQESGPGLVKPSGTLSLTCAVSGGSVSSSSWWTWVRQPPGKGLEWIGEIYHSGSTNYNPSLKSRVTISVDKSKNQFSLKLISVTAADTAVYYCARRPSGSFHFDYWGQGTLVTVSS

SEQ ID NO: 74 - protein, 9 aa, Homo sapiens
GGSVSSSSW

SEQ ID NO: 75 - protein, 7 aa, Homo sapiens
IYHSGST

SEQ ID NO: 76 - protein, 12 aa, Homo sapiens
ARRPSGSFHFDY

SEQ ID NO: 77 - protein, 111 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLLVFGGGTKLTVL

SEQ ID NO: 78 - protein, 9 aa, Homo sapiens
SSDVGGYNY

SEQ ID NO: 79 - skipped

SEQ ID NO: 80 - protein, 11 aa, Homo sapiens
SSYTSSSTLLV

SEQ ID NO: 81 - protein, 122 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTATYYCARPEHPYQYYYGMDVWGQGTTVTVSS

SEQ ID NO: 82 - protein, 8 aa, Homo sapiens
GYSFTSYW

SEQ ID NO: 83 - protein, 8 aa, Homo sapiens
IYPGDSDT

SEQ ID NO: 84 - protein, 15 aa, Homo sapiens
ARPEHPYQYYYGMDV

SEQ ID NO: 85 - protein, 110 aa, Homo sapiens
QSALTQPRSVSGSPGQSVTISCTGTSSDVGGYNYVSWYQQHPGKAPKLMIYDVSKRPSGVPDRFSGSKSGNTASLTISGLQAEDEADYYCCSYSGSYTLVFGGGTKLTVL

SEQ ID NO: 86 - protein, 9 aa, Homo sapiens
SSDVGGYNY

SEQ ID NO: 87 - skipped

SEQ ID NO: 88 - protein, 10 aa, Homo sapiens
CSYSGSYTLV

SEQ ID NO: 89 - protein, 122 aa, Homo sapiens
EVQLVQSGAEVKKPGESLKISCKGSGYRFTSYWIAWVRQMPGKGLECMGIIYPGDSDTRYSPSFQGQVTISVDKSISTAYLQWSSLKASDTAMFYCARHNNNWSLQGYFDYWGQGTLVTVSS

SEQ ID NO: 90 - protein, 8 aa, Homo sapiens
GYRFTSYW

SEQ ID NO: 91 - protein, 8 aa, Homo sapiens
IYPGDSDT

SEQ ID NO: 92 - protein, 15 aa, Homo sapiens
ARHNNNWSLQGYFDY

SEQ ID NO: 93 - protein, 112 aa, Homo sapiens
PSALTQPASVSGSPGQSITISCTGTSSDVGGYNYVSWYQQHPGKAPKLMIYDVSNRSSGVSNRFSGSKSGNTASLTISGLQAEDEADYYCSSYTGSSTSNYVFGTGTKVTVL

SEQ ID NO: 94 - protein, 9 aa, Homo sapiens
SSDVGGYNY

SEQ ID NO: 95 - skipped

SEQ ID NO: 96 - protein, 12 aa, Homo sapiens
SSYTGSSTSNYV

SEQ ID NO: 97 - protein, 122 aa, Homo sapiens
EVQLVQSGAEVKKPGESLKISCKGSGYSFTSYWIGWVRQMPGKGLEWMGIIYPGDSDTIYSPSFQGQVTISADKSISTAYLQWSSLKASDTAMYYCARQAYSSGWAAPSPYWGQGTLVTVSS

SEQ ID NO: 98 - protein, 8 aa, Homo sapiens
GYSFTSYW

SEQ ID NO: 99 - protein, 8 aa, Homo sapiens
IYPGDSDT

SEQ ID NO: 100 - protein, 15 aa, Homo sapiens
ARQAYSSGWAAPSPY

SEQ ID NO: 101 - protein, 108 aa, Homo sapiens
EIVLTQSPGTLSLSPGKRATLSCRASQSVSSSYLAWYQQKPGQAPRLLIYGASSRATGIPDRFSGSGSGTDFTLTISRLEPEDFAVYYCQQYGSSPITFGQGTRLEIK

SEQ ID NO: 102 - protein, 7 aa, Homo sapiens
QSVSSSY

SEQ ID NO: 103 - skipped

SEQ ID NO: 104 - protein, 9 aa, Homo sapiens
QQYGSSPIT

SEQ ID NO: 105 - protein, 121 aa, Homo sapiens
EVQLVESGGGLVQPGGSLRLSCAASGFTFSSYEMNWVRQAPGKGLEWVSYMSYTGSTIYYADSVKGRFTISRDNGKNSLYLQMNSLRVEDTAVYYCARGGVVVAPTYFDYWGQGTLVTVSS

SEQ ID NO: 106 - protein, 8 aa, Homo sapiens
GFTFSSYE

SEQ ID NO: 107 - protein, 8 aa, Homo sapiens
MSYTGSTI

SEQ ID NO: 108 - protein, 14 aa, Homo sapiens
ARGGVVVAPTYFDY

SEQ ID NO: 109 - protein, 111 aa, Homo sapiens
QSVLTQPPSVSGAPGQRVTISCTGSSSNIGAGYDVHWYQQLPGTAPKLLIYHNNNRPSGVPDRFSASKSGTSASLAITGLQAEDEADYYCQSYDSSLSGWVFGGGTKLTVL

SEQ ID NO: 110 - protein, 9 aa, Homo sapiens
SSNIGAGYD

SEQ ID NO: 111 - skipped

SEQ ID NO: 112 - protein, 11 aa, Homo sapiens
QSYDSSLSGWV

SEQ ID NO: 113 - protein, 120 aa, Homo sapiens
QVQLAQSGAEVKKPGASVKVSCKASGYIFTSYGISWVRQAPGQGLEWMGWISAYNSNTNYAQKLQGRVTMTTDTSTSTAYMELRSLRSDDTAVYYCARDGSRYPAYFDYWGQGTLVTVSS

SEQ ID NO: 114 - protein, 8 aa, Homo sapiens
GYIFTSYG

SEQ ID NO: 115 - protein, 8 aa, Homo sapiens
ISAYNSNT

SEQ ID NO: 116 - protein, 13 aa, Homo sapiens
ARDGSRYPAYFDY

SEQ ID NO: 117 - protein, 107 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYDSSPTFGQGTKVEIK

SEQ ID NO: 118 - protein, 7 aa, Homo sapiens
QSVSSSY

SEQ ID NO: 119 - skipped

SEQ ID NO: 120 - protein, 8 aa, Homo sapiens
QQYDSSPT

SEQ ID NO: 121 - protein, 127 aa, Homo sapiens
QITLKESGPTLVKPTQTLTLTCTFSGFSLSTSEVGVGWIRQPPGKALEWLALIYWDDDKRYSPSLKSRLTITKDTSKNQVVLTMTNMDPVDTATYYCAHIKQDCSSTSCYPVTFDYWGQGTLVTVSS

SEQ ID NO: 122 - protein, 10 aa, Homo sapiens
GFSLSTSEVG

SEQ ID NO: 123 - protein, 7 aa, Homo sapiens
IYWDDDK

SEQ ID NO: 124 - protein, 19 aa, Homo sapiens
AHIKQDCSSTSCYPVTFDY

SEQ ID NO: 125 - protein, 113 aa, Homo sapiens
DIVMTQSPDSLAVSLGERATINCKSSQSVLDRSNNKKYLAWYQQKSGQPPKLLIYWASTRESGVPDRFSGSGSGTDFTLTISSLQAEDVAVYYCQQYYSTPWTFGQGTKVEIK

SEQ ID NO: 126 - protein, 12 aa, Homo sapiens
QSVLDRSNNKKY

SEQ ID NO: 127 - skipped

SEQ ID NO: 128 - protein, 9 aa, Homo sapiens
QQYYSTPWT

SEQ ID NO: 129 - protein, 138 aa, Homo sapiens
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCARGGQDEYTSWGVRVGTRRATDRDYYYGMDVWGQGTTVTVSS

SEQ ID NO: 130 - protein, 8 aa, Homo sapiens
GFTFSSYE

SEQ ID NO: 131 - protein, 8 aa, Homo sapiens
ISSSGSTI

SEQ ID NO: 132 - protein, 31 aa, Homo sapiens
ARGGQDEYTSWGVRVGTRRATDRDYYYGMDV

SEQ ID NO: 133 - protein, 107 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNTYPLTFGGGTKVEIK

SEQ ID NO: 134 - protein, 6 aa, Homo sapiens
QSISSW

SEQ ID NO: 135 - skipped

SEQ ID NO: 136 - protein, 9 aa, Homo sapiens
QQYNTYPLT

SEQ ID NO: 137 - protein, 129 aa, Homo sapiens
EVQLVESGGGLVQPGGSLRLSCAASGFTFSSYEMNWVRQAPGKGLEWVSYISSSDSTIFYADSVKGRFTISRDNAKNSLYLQMNSLRAEDTAVYYCARGDPECSGGSCYLWYYYGMDVWGQGTTVTVSS

SEQ ID NO: 138 - protein, 8 aa, Homo sapiens
GFTFSSYE

SEQ ID NO: 139 - protein, 8 aa, Homo sapiens
ISSSDSTI

SEQ ID NO: 140 - protein, 22 aa, Homo sapiens
ARGDPECSGGSCYLWYYYGMDV

SEQ ID NO: 141 - protein, 110 aa, Homo sapiens
QSVLTQPPSASGTPGQRVTISCSGGSSNIGSNYVYWYQHLPGTAPKLLIYRNNQRPSGVPDRFSGSKSGTSASLAISGLRSEDEADYYCAVWDDSLSGWVFGGGTKLTVL

SEQ ID NO: 142 - protein, 8 aa, Homo sapiens
SSNIGSNY

SEQ ID NO: 143 - skipped

SEQ ID NO: 144 - protein, 11 aa, Homo sapiens
AVWDDSLSGWV

SEQ ID NO: 145 - protein, 120 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGGKWELSDFDYWGQGTLVTVSS

SEQ ID NO: 146 - protein, 8 aa, Homo sapiens
GFTFSSYA

SEQ ID NO: 147 - protein, 8 aa, Homo sapiens
ISYDGSNK

SEQ ID NO: 148 - protein, 13 aa, Homo sapiens
ARGGKWELSDFDY

SEQ ID NO: 149 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSLFTFGPGTKVDIK

SEQ ID NO: 150 - protein, 7 aa, Homo sapiens
QSVSSSY

SEQ ID NO: 151 - skipped

SEQ ID NO: 152 - protein, 9 aa, Homo sapiens
QQYGSSLFT

SEQ ID NO: 153 - protein, 126 aa, Homo sapiens
QVQLVESGGGVVQPGRSLRLSCAASGFTFSSFAMHWVRQAPGKGLEWVAVISYVGSKKYNADSVKGRFTISRDNSKNTLYLQMNSLRAEDTAVYYCARELEDYYDSSGYYYGMDVWGQGTTVTVSS

SEQ ID NO: 154 - protein, 8 aa, Homo sapiens
GFTFSSFA

SEQ ID NO: 155 - protein, 8 aa, Homo sapiens
ISYVGSKK

SEQ ID NO: 156 - protein, 19 aa, Homo sapiens
ARELEDYYDSSGYYYGMDV

SEQ ID NO: 157 - protein, 107 aa, Homo sapiens
DIQLTQSPSFLSASVGDRVTITCRASQGISSYLAWYQQKPGKAPKLLIYAASTLQSGVPSRFSGSGSGTEFTLTISSLQPEDFATYYCQQLNSYPRTFGPGTKVDIK

SEQ ID NO: 158 - protein, 6 aa, Homo sapiens
QGISSY

SEQ ID NO: 159 - skipped

SEQ ID NO: 160 - protein, 9 aa, Homo sapiens
QQLNSYPRT

SEQ ID NO: 161 - protein, 121 aa, Homo sapiens
QVQLVESGGGVVQPGRSLRLSCAASGFTFSSYSMHWVRQAPGKGLEWVAVISYDGSNKYYEDSVKGRFTISRDNSKNTLYLQMHSLRAEDTAVYYCARDKWFGDLNPFDPWGQGTLVTVSS

SEQ ID NO: 162 - protein, 8 aa, Homo sapiens
GFTFSSYS

SEQ ID NO: 163 - protein, 8 aa, Homo sapiens
ISYDGSNK

SEQ ID NO: 164 - protein, 14 aa, Homo sapiens
ARDKWFGDLNPFDP

SEQ ID NO: 165 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGLGTKVEIK

SEQ ID NO: 166 - protein, 7 aa, Homo sapiens
QSVSSSY

SEQ ID NO: 167 - skipped

SEQ ID NO: 168 - protein, 9 aa, Homo sapiens
QQYGSSPWT

SEQ ID NO: 169 - protein, 126 aa, Homo sapiens
QVQLVQSGAEVKKPGSSVKVSCKAFGGTFSSSAITWVRQAPGQGLEWMGRIIPIFGTANYAQKFQGRVTITADKSTTTAYMELSSLRSEDTAVYYCALKADSYCSGSSCYPSFDYWGQGTLVTVSS

SEQ ID NO: 170 - protein, 8 aa, Homo sapiens
GGTFSSSA

SEQ ID NO: 171 - protein, 8 aa, Homo sapiens
IIPIFGTA

SEQ ID NO: 172 - protein, 19 aa, Homo sapiens
ALKADSYCSGSSCYPSFDY

SEQ ID NO: 173 - protein, 108 aa, Homo sapiens
SYVLTQPPSVSVAPGKTARITCGGNNIGSKSVHWYQQRPGQAPVLVVYDDSDRPSGIPERFSGSNSGNTATLTISRVEAGDEADYYCQVWDSSSDQEVFGGGTKLTVL

SEQ ID NO: 174 - protein, 6 aa, Homo sapiens
NIGSKS

SEQ ID NO: 175 - skipped

SEQ ID NO: 176 - protein, 11 aa, Homo sapiens
QVWDSSSDQEV

SEQ ID NO: 177 - protein, 126 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARHSYCSGGSCYLAPLGDWGQGTLVTVSS

SEQ ID NO: 178 - protein, 10 aa, Homo sapiens
GGSISSSSYY

SEQ ID NO: 179 - protein, 7 aa, Homo sapiens
IYYSGST

SEQ ID NO: 180 - protein, 18 aa, Homo sapiens
ARHSYCSGGSCYLAPLGD

SEQ ID NO: 181 - protein, 108 aa, Homo sapiens
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDFYVFGTGTKVTVL

SEQ ID NO: 182 - protein, 6 aa, Homo sapiens
NIGSKS

SEQ ID NO: 183 - skipped

SEQ ID NO: 184 - protein, 11 aa, Homo sapiens
QVWDSSSDFYV

SEQ ID NO: 185 - protein, 121 aa, Homo sapiens
QVQMVESGGGVVQPGRSLRLSCAASGFTFSSYPIHWVRQVPGKGLEWVAVISYDGSNKYYADSVKGRFTISRDNSKNTLYLQMNSLRPEDTAVYFCARGRGDFELNSFDYWGQGTLVTVSS

SEQ ID NO: 186 - protein, 8 aa, Homo sapiens
GFTFSSYP

SEQ ID NO: 187 - protein, 8 aa, Homo sapiens
ISYDGSNK

SEQ ID NO: 188 - protein, 14 aa, Homo sapiens
ARGRGDFELNSFDY

SEQ ID NO: 189 - protein, 108 aa, Homo sapiens
EIVLTQSPGTLSLSPGERATLSCRASQSVSSSYLGWYQQKLGQAPRLLIYGASSRATGIPDRFSGSGSGTDFTLTISRLEPEDFAVYYCQQYGSTPFTFGPGTKVDIK

SEQ ID NO: 190 - protein, 7 aa, Homo sapiens
QSVSSSY

SEQ ID NO: 191 - skipped

SEQ ID NO: 192 - protein, 9 aa, Homo sapiens
QQYGSTPFT

SEQ ID NO: 193 - protein, 119 aa, Homo sapiens
QVQLVESGGGVVQPGRSLRLSCAASGFTFSSYAMHWVRQAPGKGLEWVAIISYDGSNKYYADSVKGRFTISRDNSKNTLYFRMNSLRAEDTAVYYCARAYTELSSLDYWGQGTLVTVSS

SEQ ID NO: 194 - protein, 8 aa, Homo sapiens
GFTFSSYA

SEQ ID NO: 195 - protein, 8 aa, Homo sapiens
ISYDGSNK

SEQ ID NO: 196 - protein, 12 aa, Homo sapiens
ARAYTELSSLDY

SEQ ID NO: 197 - protein, 108 aa, Homo sapiens
EIVLTQSPGTLSLSPGERATLSCRASQSFSSSYLAWYQQKPGQAPRLVIYGASSRATGIPDRFSGSGSGTDFTLTISRLEPEDFRVYYCQQFGSTPFTFGPGTKVDIK

SEQ ID NO: 198 - protein, 7 aa, Homo sapiens
QSFSSSY

SEQ ID NO: 199 - skipped

SEQ ID NO: 200 - protein, 9 aa, Homo sapiens
QQFGSTPFT

SEQ ID NO: 201 - protein, 123 aa, Homo sapiens
EVQLVQSGAEVKKPGESLNISCKTSGYSFTSYWIGWVRQMPGKGLEWMGIIYPGDSDTRYSPSFQGQVTISADKSITTAYLQWSSLKASDTAMYYCARFKGVGATTLGWFDPWGQGTLVTVSS

SEQ ID NO: 202 - protein, 8 aa, Homo sapiens
GYSFTSYW

SEQ ID NO: 203 - protein, 8 aa, Homo sapiens
IYPGDSDT

SEQ ID NO: 204 - protein, 16 aa, Homo sapiens
ARFKGVGATTLGWFDP

SEQ ID NO: 205 - protein, 110 aa, Homo sapiens
QSALTQPRSVSGSPGQSVTISCTGTSSDVGGYNYVSWYQQHPGKAPKLMIYDVSKRPSGVPDRFSGSKSGNTASLTISGLQAEDEAEYYCCSYTGSYTLVFGGGTKLTVL

SEQ ID NO: 206 - protein, 9 aa, Homo sapiens
SSDVGGYNY

SEQ ID NO: 207 - skipped

SEQ ID NO: 208 - protein, 10 aa, Homo sapiens
CSYTGSYTLV

SEQ ID NO: 209 - protein, 121 aa, Homo sapiens
EVQLVQSGAEVKKPGESLKISCKGSGYSFTSYWIGWVRQMPGKGLEWMGIIYPGDSDTRYSPSFQGQVSISADKSISTAYLQWSSLKASDTAMYYCARSIPPYYPFYFDYWGQGTLVTVSS

SEQ ID NO: 210 - protein, 8 aa, Homo sapiens
GYSFTSYW

SEQ ID NO: 211 - protein, 8 aa, Homo sapiens
IYPGDSDT

SEQ ID NO: 212 - protein, 14 aa, Homo sapiens
ARSIPPYYPFYFDY

SEQ ID NO: 213 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYTSSPNTFGQGTKLEIK

SEQ ID NO: 214 - protein, 7 aa, Homo sapiens
QSVSSSY

SEQ ID NO: 215 - skipped

SEQ ID NO: 216 - protein, 9 aa, Homo sapiens
QQYTSSPNT

SEQ ID NO: 217 - protein, 138 aa, Homo sapiens
EVQLVESGGGLVQPGGSLRLSCAASGFTFSSYEMNWVRQAPGKGLEWVSYISSSGSTIYYAESVKGRFTISRDNAKNSLYLQMNSLRAEDTAVYYCARGGQDEYSSWGVRVGTRRATARDYYYEMDVWGQGTTVTVSS

SEQ ID NO: 218 - protein, 8 aa, Homo sapiens
GFTFSSYE

SEQ ID NO: 219 - protein, 8 aa, Homo sapiens
ISSSGSTI

SEQ ID NO: 220 - protein, 31 aa, Homo sapiens
ARGGQDEYSSWGVRVGTRRATARDYYYEMDV

SEQ ID NO: 221 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDIATYYCQQSYSTPRTFGQGTKVEIK

SEQ ID NO: 222 - protein, 6 aa, Homo sapiens
QSISSY

SEQ ID NO: 223 - skipped

SEQ ID NO: 224 - protein, 9 aa, Homo sapiens
QQSYSTPRT

SEQ ID NO: 225 - protein, 126 aa, Homo sapiens
EVQLVESGGGLVQPGGSLRLSCAASGFTFSSFEMNWVRQAPGKGLEWVSDISSSGSTIYYADSVKGRFSISRDNAKNSLYLQMNSLRAEDTAVYYCARGACGSGGSCYQTGDLDYWGQGTLVTVSS

SEQ ID NO: 226 - protein, 8 aa, Homo sapiens
GFTFSSFE

SEQ ID NO: 227 - protein, 8 aa, Homo sapiens
ISSSGSTI

SEQ ID NO: 228 - protein, 19 aa, Homo sapiens
ARGACGSGGSCYQTGDLDY

SEQ ID NO: 229 - protein, 110 aa, Homo sapiens
QAVVTQEPSLTVSPGGTVTLTCGSSSGAVTSGHYPYWFQQKPGQAPRTLIYHTSNKHSWTPARFSGSLLGGKAALTLSGAQPEDEAEYYCLLSYSGAWRVFGGGTKLTVL

SEQ ID NO: 230 - protein, 9 aa, Homo sapiens
SGAVTSGHY

SEQ ID NO: 231 - skipped

SEQ ID NO: 232 - protein, 10 aa, Homo sapiens
LLSYSGAWRV

SEQ ID NO: 233 - protein, 119 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARGPGWGGDLDVWGKGTTVTVSS

SEQ ID NO: 234 - protein, 8 aa, Homo sapiens
GFTFSSYG

SEQ ID NO: 235 - protein, 8 aa, Homo sapiens
IWYDGSNK

SEQ ID NO: 236 - protein, 11 aa, Homo sapiens
ARGGQDEYSSW

SEQ ID NO: 237 - protein, 106 aa, Homo sapiens
SYELTQPLSVSVALGQTARITCGGNNIGSKNVHWYQQKPGQAPVMVIYRDSNRPSGIPERFSGSNSGNTATLTISRAQAGDEADYYCQVWDTSTGVFGGGTKLTVL

SEQ ID NO: 238 - protein, 6 aa, Homo sapiens
NIGSKN

SEQ ID NO: 239 - skipped

SEQ ID NO: 240 - protein, 9 aa, Homo sapiens
QVWDTSTGV

SEQ ID NO: 241 - protein, 125 aa, Homo sapiens
QVQLVQSGAEVKKPGASVKVSCKASGYTFTGYYMHWVRQAPGQGLEWMGWINPNSGVTNYAQRFQGRVTMTRDTSISTASMELSRLRSDDTAVYYCARDDRRIVAGADYYAMDVWGQGTTVTVSS

SEQ ID NO: 242 - protein, 8 aa, Homo sapiens
GYTFTGYY

SEQ ID NO: 243 - protein, 8 aa, Homo sapiens
INPNSGVT

SEQ ID NO: 244 - protein, 18 aa, Homo sapiens
ARDDRRIVAGADYYAMDV

SEQ ID NO: 245 - protein, 106 aa, Homo sapiens
SYELTQPPSVSVSPGQTASITCSGDKLGDKYACWYQQRPGQSPVLVIYQDDKRPSGIPERFSGSNSGNAATLTISGTQAIDEADYYCQAWDSSTVVFGGGTKLTVL

SEQ ID NO: 246 - protein, 6 aa, Homo sapiens
KLGDKY

SEQ ID NO: 247 - skipped

SEQ ID NO: 248 - protein, 9 aa, Homo sapiens
QAWDSSTVV

SEQ ID NO: 249 - protein, 122 aa, Homo sapiens
EVQLEESGGGLVQPGGSLRLSCAASGFTFSSYWMHWVRQAPGKGLVWVSRINSDGSSTTYADSVKGRFTISRDNAKSTLYLQMNSLRAEDTAVYYCARGGSYHYSYYFMDVWGKGTTVTVSS

SEQ ID NO: 250 - protein, 8 aa, Homo sapiens
GFTFSSYW

SEQ ID NO: 251 - protein, 8 aa, Homo sapiens
INSDGSST

SEQ ID NO: 252 - protein, 15 aa, Homo sapiens
ARGGSYHYSYYFMDV

SEQ ID NO: 253 - protein, 107 aa, Homo sapiens
SYELTQPPSVSVSPGQTASITCSGDKLGNKYACWYQQKPGQSPVLVICEDRKRPSGIPERFSGSNSGNTATLTISGTQAMDEADYYCQAWDSSTALVFGGGTKLTVL

SEQ ID NO: 254 - protein, 6 aa, Homo sapiens
KLGNKY

SEQ ID NO: 255 - skipped

SEQ ID NO: 256 - protein, 10 aa, Homo sapiens
QAWDSSTALV

SEQ ID NO: 257 - protein, 120 aa, Homo sapiens
QVQLVESGGGVVQPGRSLRLSCAASGFTFSSYAVHWVRQAPGKGLEWVAVISYDGSNKYYADSVKGRFTISRDNSKNTLSLQMNSLRAEDTAVYHCARGGAVEVAASDYWGQGTLVTVSS

SEQ ID NO: 258 - protein, 8 aa, Homo sapiens
GFTFSSYA

SEQ ID NO: 259 - protein, 8 aa, Homo sapiens
ISYDGSNK

SEQ ID NO: 260 - protein, 13 aa, Homo sapiens
ARGGAVEVAASDY

SEQ ID NO: 261 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGTTPFTFGPGTKVDIK

SEQ ID NO: 262 - protein, 7 aa, Homo sapiens
QSVSSSY

SEQ ID NO: 263 - skipped

SEQ ID NO: 264 - protein, 9 aa, Homo sapiens
QQYGTTPFT

SEQ ID NO: 265 - protein, 118 aa, Homo sapiens
QVQLVESGGGVVQPGRSLRLSCAASGFTFSSYGMHWVRQAPGKGLEWLAVISYDGSNKYYADSVKGRFTISRDNSKTTLFLQMNSLRAEDTAVYYCAKDIQRRLIDYWGQGTLVTVSS

SEQ ID NO: 266 - protein, 8 aa, Homo sapiens
GFTFSSYG

SEQ ID NO: 267 - protein, 8 aa, Homo sapiens
ISYDGSNK

SEQ ID NO: 268 - protein, 11 aa, Homo sapiens
AKDIQRRLIDY

SEQ ID NO: 269 - protein, 107 aa, Homo sapiens
DIQMTQSPSSLSASVGDRVTITCQASQDISNFLNWYQQKPRKAPKLLIFDASNLETGVPSRFSGSGSGTDFTFTISSLQPEDIATYYCQQYDNLPLTFGGGTKVEIK

SEQ ID NO: 270 - protein, 6 aa, Homo sapiens
QDISNF

SEQ ID NO: 271 - skipped

SEQ ID NO: 272 - protein, 9 aa, Homo sapiens
QQYDNLPLT

SEQ ID NO: 273 - protein, 120 aa, Homo sapiens
EEQLVESGGGLVQPGGSLRLSCAASGFTFSSYSMNWVRQAPGKGLEWVSYISSSSSTIHYADSVKGRFTISRDNAKNSLYLQMNSLRAEDTAVYYCAREGNSGYEPLDYWGQGTLVTVSS

SEQ ID NO: 274 - protein, 8 aa, Homo sapiens
GFTFSSYS

SEQ ID NO: 275 - protein, 8 aa, Homo sapiens
ISSSSSTI

SEQ ID NO: 276 - protein, 13 aa, Homo sapiens
AREGNSGYEPLDY

SEQ ID NO: 277 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVEIK

SEQ ID NO: 278 - protein, 6 aa, Homo sapiens
QSISSY

SEQ ID NO: 279 - skipped

SEQ ID NO: 280 - protein, 9 aa, Homo sapiens
QQSYSTPWT

SEQ ID NO: 281 - protein, 119 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARSPWELDAFDIWGQGTMVTVSS

SEQ ID NO: 282 - protein, 8 aa, Homo sapiens
GFTFSSYA

SEQ ID NO: 283 - protein, 8 aa, Homo sapiens
ISYDGSNK

SEQ ID NO: 284 - protein, 12 aa, Homo sapiens
ARSPWELDAFDI

SEQ ID NO: 285 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSQITFGPGTKVDIK

SEQ ID NO: 286 - protein, 7 aa, Homo sapiens
QSVSSSY

SEQ ID NO: 287 - skipped

SEQ ID NO: 288 - protein, 9 aa, Homo sapiens
QQYGSSQIT

SEQ ID NO: 289 - protein, 128 aa, Homo sapiens
QVQLQESGPGLVKPSQTLSLTCTVSGGSISSDDYYWTWIRLPPGKGLEWIGYIFYTGGTYYNPSLKSRVTISLDRSKNQFSLKLSSVTAADTAVYFCARAPETYCSTTNCYKGYFDSWGQGTLVTVSS

SEQ ID NO: 290 - protein, 10 aa, Homo sapiens
GGSISSDDYY

SEQ ID NO: 291 - protein, 7 aa, Homo sapiens
IFYTGGT

SEQ ID NO: 292 - protein, 20 aa, Homo sapiens
ARAPETYCSTTNCYKGYFDS

SEQ ID NO: 293 - protein, 107 aa, Homo sapiens
DIQMTQSPSSVSASVGDRVTITCRASQGISSWLGWYQQKPGKAPKLLIYAASSLQSGVPSRFSGSGSGTDFTLTISSLQPEDFATYYCQQANSFPRTFGQGTKVEIK

SEQ ID NO: 294 - protein, 6 aa, Homo sapiens
QGISSW

SEQ ID NO: 295 - skipped

SEQ ID NO: 296 - protein, 9 aa, Homo sapiens
QQANSFPRT

SEQ ID NO: 297 - protein, 127 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARDTPYCSSTSCYMGDFDYWGQGTLVTVSS

SEQ ID NO: 298 - protein, 10 aa, Homo sapiens
GGSISSSSYY

SEQ ID NO: 299 - protein, 7 aa, Homo sapiens
IYYSGST

SEQ ID NO: 300 - protein, 19 aa, Homo sapiens
ARDTPYCSSTSCYMGDFDY

SEQ ID NO: 301 - protein, 109 aa, Homo sapiens
DIQMTQSPSSLSASVGDRVTITCRASQSISSYLNWYQQKLGKAPKLLIYAASSLQSGVPSRFSGSGSGTDFTLTISSLQPEDFATYYCQQSYSTPPLFTFGPGTKVDIK

SEQ ID NO: 302 - protein, 6 aa, Homo sapiens
QSISSY

SEQ ID NO: 303 - skipped

SEQ ID NO: 304 - protein, 11 aa, Homo sapiens
QQSYSTPPLFT

SEQ ID NO: 305 - DNA, 20 bp, Synthetic construct
  source 1..20: mol_type = other DNA
tgccattccagttatgtgcc

SEQ ID NO: 306 - DNA, 20 bp, Synthetic construct
  source 1..20: mol_type = other DNA
cacgcatagcaccacgatta